(19) United States
(12) Patent Application Publication
(10) Pub. No.: US 2013/0309692 A1
(43) Pub. Date: Nov. 21, 2013

(54) MARKERS FOR DETECTING HUMAN FOLLICULAR HELPER T CELLS AND METHOD FOR DETECTING HUMAN FOLLICULAR HELPER T CELLS

(71) Applicant: Sysmex Corporation, South San Francisco, CA (US)

(72) Inventors: Masatoshi YANAGIDA, Kobe-shi (JP); Hitoshi UGA, Kobe-shi (JP); Takahiro OKAZAWA, Kobe-shi (JP); Yoshiaki MIYAMOTO, Kobe-shi (JP); Masafumi IKEDA, Kobe-shi (JP); Hirokazu KURATA, Kobe-shi (JP)

(73) Assignee: SYSMEX CORPORATION, Kobe-shi (JP)

(21) Appl. No.: 13/891,989

(22) Filed: May 10, 2013

Related U.S. Application Data
(63) Continuation of application No. PCT/JP2011/075856, filed on Nov. 9, 2011.

(30) Foreign Application Priority Data
Nov. 11, 2010 (JP) 2010-253140

(51) Int. Cl. G01N 33/68

(52) U.S. Cl. CPC G01N 33/6893
USPC 435/7.24; 536/23.5; 530/350

(57) ABSTRACT

The present invention relates to polynucleotide markers and protein markers for detecting human follicular helper T cells. The present invention also relates to methods for detecting human follicular helper T cells using the markers.

### CROSS-REFERENCE TO RELATED APPLICATION

This application is a continuation of International Application PCT/JP2011/075856 filed on Nov. 9, 2011, which claims benefit of Japanese patent application JP 2010-253140 filed on Nov. 11, 2010, both of which are incorporated herein by reference in their entireties.

### BACKGROUND OF THE INVENTION

The present invention relates to markers for detecting human follicular helper T cells. The present invention also relates to methods for detecting human follicular helper T cells using these markers.

Follicular helper T cells (hereinafter also referred to as “Tfh cells”) were discovered in follicular germinal center of secondary lymphoid tissue such as lymph node and spleen as helper T cells which activate B cells and promote antibody production. Tfh cells are also known as cells which express a chemokine receptor CXCR5 (chemokine (C-X-C motif) receptor 5) and secrete a cytokine IL-21 (interleukin 21) (see King C. et al., “T follicular helper (TFH) cells in normal and dysregulated immune responses”, Annu. Rev. Immunol., vol. 26, p. 741-766 (2008); and Spolski R. et al., “IL-21 and T follicular helper cells”, Int. Immunol., vol. 22, p. 7-12 (2009)).

Tfh cells are generally useful for prevention of infection. It is believed however that, in autoimmune diseases such as systemic lupus erythematosus (SLE), Tfh cells provide inappropriate activation signal to autoreactive B cells to allow production of autoantibodies (see King C. et al., supra). There are some reports on that, in autoimmune diseases such as SLE and Sjogren's syndrome, Tfh cells may be exuded from lymphatic tissue to peripheral blood. Namely, the result obtained by measuring Tfh cells may be used for diagnoses and examinations of diseases associated with Tfh cells such as autoimmune diseases, e.g. SLE or Sjogren's syndrome.

One of the methods for detecting Tfh cells utilizes marker molecules which are specifically expressed in Tfh cells. For example, Chtanova T. et al. (“T follicular cells express a distinctive transcriptional profile, reflecting their role as non-Th1/Th2 effector cells that provide help for B cells”, J. Immunol., vol. 173, p. 68-78 (2004)) found genes specifically expressed in Tfh cells by carrying out gene expression assay of Tfh cells isolated from human tonsil, cultured Th1 and Th2 cells from human umbilical cord blood and memory T cells (effector memory T cells (TEM) and central memory T cells (T_{CM})) isolated from human peripheral blood. WO 2010/003002 discloses that genes specifically expressed in Tfh cells were found by gene expression assay of Tfh cells isolated from murine splenocytes and cultured Th1, Th2 and Th17 cells.

### BRIEF SUMMARY OF THE INVENTION

Chtanova T. et al. compared Th1, Th2 cells and memory T cells with Tfh cells in the gene expression assay and did not compare Tfh cells with other Th cells (for example, Th9, Th17 and Th22 cells) (see Chtanova T. et al., supra). The genes described in WO 2010/003002 as being expressed in Tfh cells may have different gene expression profile from human Tfh cells as they were identified from murine Tfh cells. In the gene expression assay carried out in this document, Th1, Th2 cells and Th17 cells were compared with Tfh cells but Tfh cells were not compared with other Th cells (for example, Th9 and Th22 cells).

Thus the present inventors aimed to identify genes specifically expressed in human Tfh cells compared with other Th cells (Th1, Th2, Th9, Th17, Th22 and Treg cells) by using Tfh cells isolated from human peripheral blood and cultured under optimal conditions and to provide the identified genes as new molecular markers for allowing detection of human Tfh cells.

The present inventors first isolated Tfh cells from peripheral blood of healthy adults and cultured the cells. The present inventors then identified the genes specifically expressed in the obtained Tfh cells to complete the present invention.

Thus the present invention provides a polynucleotide marker for detecting human Tfh cells, which is a polynucleotide having a base sequence of at least one gene selected from:

a gene encoding a membrane protein represented by CD9 (CD9 molecule), TM4SF1 (transmembrane 4 L six family member 1), IL23R (interleukin 23 receptor), ART3 (ADP-ribosyltransferase 3), ELOVL7 (ELOVL family member 7, elongation of long chain fatty acids), KCNS3 (potassium voltage-gated channel, delayed-rectifier, subfamily S, member 3), LHFP (lipoma HMGIC fusion partner), PAWR (PRKC, apoptosis, WT1, regulator), THBS1 (thrombospondin 1), B4GALT6 (UDP-Gal:betaGlcNAc beta 1,4-galactosyltransferase, polypeptide 6), C3orf52 (chromosome 3 open reading frame 52), CD28 (CD28 molecule), CDH3 (cadherin 3, type 1, P-cadherin), CLIC2 (chloride intracellular channel 2), EMP2 (epithelial membrane protein 2), EPHA4 (EPH receptor A4), FAM26F (Family with sequence similarity 26, member F), FCGR1B (Fc fragment of IgG, high affinity Ib, receptor (CD64)), FLT1 (fms-related tyrosine kinase 1), FUT7 (fucosyltransferase 7 (alpha (1,3) fucosyltransferase)), GABBR1 (gamma-aminobutyric acid (GABA) B receptor, 1), UBD (ubiquitin D), GAP43 (growth associated protein 43), GPC4 (glypican 4), GPR26 (G protein-coupled receptor 26), GPR84 (G protein-coupled receptor 84), IL12RB2 (interleukin 12 receptor, beta 2), KIAA1244 (KIAA1244), KISS1R (KISS1 receptor), KITLG (KIT ligand), LST1 (leukocyte specific transcript 1), MAP1B (microtubule-associated protein 1B), MARS (methionyl-tRNA synthetase), NTRK3 (neurotrophic tyrosine kinase, receptor, type 3), PLSCR1 (phospholipid scramblase 1), PPP2CB (protein phosphatase 2 (formerly 2A), catalytic subunit, beta isoform), RHOU (ras homolog gene family, member U), SGMS2 (sphingomyelin synthase 2), SLC35E4 (solute carrier family 35, member E4), SORBS1 (sorbin and SH3 domain containing 1), TAC1 (tachykinin, precursor 1), TMCC2 (transmembrane and coiled-coil domain family 2), TMEM213 (transmembrane protein 213), TMTC1 (transmembrane and tetratricopeptide repeat containing 1) or TUSC3 (tumor suppressor candidate 3);

a gene encoding an extracellular/secretory protein represented by APOD (apolipoprotein D), CXCL9 (chemokine (C-X-C motif) ligand 9), CXCL10 (chemokine (C-X-C motif) ligand 10), CXCL11 (chemokine (C-X-C motif) ligand 11), FGF2 (fibroblast growth factor 2 (basic)), IL2 (interleukin 2), PTHLH (parathyroid hormone-like hormone), SERPING1 (serpin peptidase inhibitor, Glade G (C1 inhibitor), member 1), CCL 18 (chemokine (C-C motif) ligand 18 (pulmonary and activation-regulated)), CCL2 (chemokine (C-C motif) ligand 2), COL6A2 (collagen, type VI, alpha 2), CSF2 (colony stimulating factor 2), FGF1 (fibroblast growth factor 1 (acidic)), FGF18 (fibroblast growth factor 18), IFNB1 (interferon, beta 1, fibroblast), IL8 (interleukin 8), INHBA (inhibin, beta A), MDK (midkine (neurite growth-promoting factor 2)), MMP12 (matrix metallopeptidase 12 (macrophage elastase)), PPIA (peptidylprolyl isomerase A (cyclophilin A)), PRG4 (proteoglycan 4), SPARC (secreted protein, acidic, cysteine-rich (osteonectin)), STC2 (stanniocalcin 2) or TNFRSF11B (tumor necrosis factor receptor superfamily, member 11 b);

a gene encoding an intracellular protein represented by ANXA3 (annexin A3), DGKI (diacylglycerol kinase, iota), EFS (embryonal Fyn-associated substrate), MYH6 (myosin, heavy chain 6, cardiac muscle, alpha), MYH7 (myosin, heavy chain 7, cardiac muscle, beta), MY05B (myosin VB), PCGF2 (polycomb group ring finger 2), PNMA2 (paraneoplastic antigen MA2), RGS20 (regulator of G-protein signaling 20), ALDH1L2 (aldehyde dehydrogenase 1 family, member L2), BSPRY (B-box and SPRY domain containing), C10orf10 (chromosome 10 open reading frame 10), CHMP4C (chromatin modifying protein 4C), DPYSL4 (dihydropyrimidinase-like 4), ELL (elongation factor RNA polymerase II), FBXO17 (F-box protein 17), SARS2 (seryl-tRNA synthetase 2, mitochondrial), FERMT2 (fermitin family homolog 2), GBP4 (guanylate binding protein 4), HIST1H1A (histone cluster 1, H1a), INSC (inscuteable homolog), IRF4 (interferon regulatory factor 4), KLF4 (Kruppel-like factor 4 (gut)), MAST4 (microtubule associated serine/threonine kinase family member 4), METTL3 (methyltransferase like 3), MGST1 (microsomal glutathione S-transferase 1), NEXN (nexilin (F actin binding protein)), PHLDA2 (pleckstrin homology-like domain, family A, member 2), PLOD2 (procollagen-lysine, 2-oxoglutarate 5-dioxygenase 2), POLR1C (polymerase (RNA) I polypeptide C, 30 kDa), RAI14 (retinoic acid induced 14), RPL10 (ribosomal protein L10), RTKN (rhotekin), S100A9 (S 100 calcium binding protein A9), SOCS3 (suppressor of cytokine signaling 3), SPHK1 (sphingosine kinase 1), STAG3 (stromalantigen 3), TEAD4 (TEA domain family member 4), TOM1L1 (target of myb1-like 1), UCHL1 (ubiquitin carboxyl-terminal esterase L1 (ubiquitin thiolesterase)), VPS53 (vacuolar protein sorting 53 homolog), WDR74 (WD repeat domain 74), XAF1 (XIAP associated factor 1), ZNF334 (zinc finger protein 334) or ZNF503 (zinc finger protein 503);

a gene represented by DNAJC12 (DnaJ (Hsp40) homolog, subfamily C, member 12), DOK5 (docking protein 5), LOC400043 (hypothetical LOC400043), MY01B (myosin IB), SPEF2 (sperm flagellar 2), YAP1 (Yes-associated protein 1, 65 kDa), C8orf47 (chromosome 8 open reading frame 47), CA13 (carbonic anhydrase XIII), CCDC77 (coiled-coil domain containing 77), CT45A1 (cancer/testis antigen family 45, member A1), CT45A2 (cancer/testis antigen family 45, member A2), CT45A3 (cancer/testis antigen family45, member A3), CT45A4 (cancer/testis antigen family 45, member A4), CT45A5 (cancer/testis antigen family 45, member A5), CT45A6 (cancer/testis antigen family 45, member A6), LOC100133581 (hypothetical protein LOC100133581), EFR3B (EFR3 homolog B), FLJ31958 (hypothetical LOC143153), GADD45B (growth arrest and DNA-damage-inducible, beta), GAS5 (growth arrest-specific 5 (non-protein coding)), KIAA0895 (KIAA0895), LMO4 (LIM domain only 4), LOC339751 (hypothetical protein LOC339751), LOC340184 (hypothetical protein LOC340184), MAK (male germ cell-associated kinase), MIR155HG (MIR155 host gene (non-protein coding)), NAPSB (napsin B aspartic peptidase pseudogene), NPHP1 (nephronophthisis 1), OSBPL6 (oxysterol binding protein-like 6), RASSF4 (Ras association (RalGDS/AF-6) domain family member 4), RIBC2 (RIB43A domain with coiled-coils 2), RIMKLA (ribosomal modification protein rimK-like family member A), SH3TC2 (SH3 domain and tetratricopeptide repeats 2), USP12 (ubiquitin specific peptidase 12) or ZBED2 (zinc finger, BED-type containing 2); and

a gene consisting of a base sequence represented by any of SEQ ID NOs: 171, 182 and 172 to 181;

or a variant or fragment thereof.

The present invention also provides a protein marker for detecting Tfh cells, which is a protein encoded by at least one gene described above or a functionally equivalent variant or fragment thereof.

The present invention further provides a method for detecting human Tfh cells comprising detecting at least one polynucleotide marker for detecting human Tfh cells or the protein marker for detecting human Tfh cells described above in a sample containing cells.

Detection of at least one polynucleotide marker or protein marker for detecting human Tfh cells of the present invention allows specific detection of human Tfh cells.

### BRIEF DESCRIPTION OF THE DRAWINGS

FIG. 1 is histograms (particle distribution) showing the percentage of CD9 positive cells within human peripheral blood-derived cultured Th1, Th2 and Tfh cells; and

FIG. 2 is a graph showing enrichment ratio of Tfh cells in the CXCR5 fractionated sample and the CXCR5/CD9 fractionated sample.

### DETAILED DESCRIPTION OF THE INVENTION

The polynucleotide marker for detecting human Tfh cells of the present invention (hereinafter also referred to as “the present polynucleotide marker”) is a polynucleotide having the base sequence of at least one gene selected from the above genes or a variant or fragment thereof.

The present polynucleotide marker is a polynucleotide which has been found to be specifically present in Tfh cells compared to other helper T cells derived from human peripheral blood (Th1, Th2, Th9, Th17 and Th22 cells and regulatory T (Treg) cells) or a variant or fragment thereof.

The present invention also encompasses the use of a polynucleotide having the base sequence of at least one gene selected from the above genes or a variant or fragment thereof, as a marker for detecting human Tfh cells.

The present polynucleotide marker is preferably a polynucleotide having the base sequence of at least one gene selected from:

a gene encoding a membrane protein represented by CD9, TM4SF1, IL23R, ART3, ELOVL7, KCNS3, LHFP, PAWR or THBS1;

a gene encoding an extracellular/secretory protein represented by APOD, CXCL9, CXCL10, CXCL11, FGF2, IL2, PTHLH or SERPING1;

a gene encoding an intracellular protein represented by ANXA3, DGKI, EFS, MYH6, MYH7, MYO5B, PCGF2, PNMA2 or RGS20;

a gene represented by DNAJC12, DOK5, LOC400043, MYO1B, SPEF2 or YAP1; and

a gene consisting of the base sequence represented by SEQ ID NO: 171 or 182;

or a variant or fragment thereof.

More preferably, the present polynucleotide marker is a polynucleotide having the base sequence of the gene encoding CD9 or a variant or fragment thereof (hereinafter referred to as “polynucleotide marker for CD9”).

As used herein, the term “gene” has the equivalent meaning as the term generally used in the art and refers to a part of a genome which is to be transcribed to mRNA and translated to a protein.

As used herein, the gene consisting of the base sequence represented by any of SEQ ID NOs: 171, 182 and 172 to 181 is a gene from which the base sequence represented by any of these SEQ ID NOs or mRNA containing a complementary base sequence thereof is transcribed. Namely, the gene consisting of the base sequence represented by any of SEQ ID NOs: 171, 182 and 172 to 181 includes a gene consisting of a complementary base sequence of the base sequence represented by any of SEQ ID NOs: 171, 182 and 172 to 181.

As used herein, a membrane protein means a protein contained in a membrane fraction of a cell. An extracellular/secretory protein means a protein synthesized intracellularly and located at outside of a cell membrane or secreted to outside of a cell membrane. An intracellular protein means a protein which is mainly located in a cell.

As used herein, the phrase “a polynucleotide is “specifically expressed” in Tfh cells” means that the expression of the polynucleotide in Tfh cells is significantly higher than the expression of the polynucleotide in cells other than Tfh cells.

Specifically, the above phrase means that the expression of the polynucleotide in Tfh cells is about three times or more of the expression of the polynucleotide in cells other than Tfh cells. Preferably, the expression of the polynucleotide in Tfh cells is about three times or more of the expression of the polynucleotide in helper T cells other than Tfh cells (Th1 cells, Th2 cells, Th9 cells, Th17 cells, Th22 and Treg cells).

The base sequences of the present polynucleotide markers per se are already known. These can be obtained from, for example, UniGene (a database provided by National Center for Biotechnology Information: NCBI). Entrez gene IDs, UniGene IDs, Protein IDs and Transcript IDs of the base sequences of the present polynucleotide markers are shown in Table 16.

The “Probe set ID” is an identification number for specifying a probe set for gene identification used in GeneChip® of Affymetrix. The Probe set IDs shown in Table 16 of the present specification are the identification numbers for probe sets mounted on the “Human Genome U133 Plus 2.0” array. The target sequences of the Probe sets are represented by SEQ ID NOs: 1 to 182.

As used herein, a “variant” of a polynucleotide means a polynucleotide containing a mutation which does not change the nature of the protein encoded by the gene. The mutation includes deletion or substitution of one or more nucleotides from or addition of one or more nucleotides to the base sequence of the above gene.

The variant has a sequence identity of generally at least 80%, preferably at least 85%, more preferably at least about 90%, still more preferably at least 95% with the base sequence of the above gene.

As used herein, the sequence identity of a base sequence and an amino acid sequence means the value calculated by using BLASTN, BLASTP, BLASTX or TBLASTN (for example, available at http://www.ncbi.nlm.nih.gov) with the default settings.

As used herein, a “fragment” of a polynucleotide means a polynucleotide having a contiguous partial sequence of the base sequence of the above gene and having a length which allows specific hybridization thereof with a probe for detecting human Tfh cells described hereinbelow.

The present polynucleotide marker may be either DNA or RNA, or any of the gene per se (DNA), mRNA, cDNA and cRNA.

It is also possible to detect human Tfh cells by detecting at least one protein encoded by the gene as the present polynucleotide marker. Thus the present invention also encompasses the protein encoded by at least one gene described above. Namely, the protein marker for detecting human Tfh cells of the present invention (hereinafter also referred to as the “present protein marker”) is a protein encoded by at least one gene selected from the above genes or a functionally equivalent variant or fragment thereof.

Amino acid sequences of the protein markers can be obtained based on the base sequences of the polynucleotide markers retrieved from UniGene described above and the like. The amino acid sequences can also be obtained from databases provided by NCBI or the like as described above. The Protein ID numbers from NCBI for the amino acid sequences of the present protein markers are shown in Table 16.

A functionally equivalent variant of a protein means a protein containing a mutation which does not change the function of the protein. The mutation includes deletion or substitution of one or more amino acids from or addition of one or more amino acids to the known amino acid sequence of the above protein.

The functionally equivalent variant of the protein has a sequence identity of generally at least 80%, preferably at least 85%, more preferably at least about 90%, still more preferably at least 95% with the known amino acid sequence of the above protein.

A fragment of a protein means a polypeptide having a contiguous partial sequence of the known amino acid sequence of the above protein and having a length which allows specific recognition by an antibody or nucleic acid aptamer for detecting human Tfh cells described hereinbelow.

In a preferred embodiment of the present invention, the present protein marker is a protein having an amino acid sequence of CD9 or a functionally equivalent variant or fragment thereof (hereinafter referred to as the “protein marker for CD9”).

The present invention also encompasses the use of a protein encoded by the gene as the present polynucleotide marker or a functionally equivalent variant or fragment thereof, as a marker for detecting human Tfh cells.

A molecule which can specifically hybridize to the present polynucleotide marker is useful as a probe for detecting human Tfh cells because it can be used for detecting the marker. The probe may be any of nucleic acid probes such as DNA or RNA and peptide probes which can specifically hybridize to the present polynucleotide marker. The probe for detecting human Tfh cells is particularly preferably a nucleic acid probe, particularly a DNA probe for detecting the polynucleotide marker.

As used herein, the phrase “a molecule “can specifically hybridize”” means the molecule can hybridize to a target nucleic acid molecule (the above polynucleotide marker) under the stringent condition.

As used herein, the stringent condition means a condition which allows hybridization of the probe for detecting human Tfh cells to the target polynucleotide marker with an extent detectably higher than the hybridization thereof to a polynucleotide other than the target polynucleotide marker (for example, at least two times higher than the background).

The stringent condition usually varies depending on sequences and various environments. Generally, the stringent condition is selected so that it is about 5° C. lower than a thermal melting point (Tm) of a predetermined sequence under a predetermined ionic strength and pH. The Tm is a temperature at which 50% of probes complementary to the base sequence of the target nucleic acid molecule hybridize in equilibrium to the target nucleic acid molecules (under a predetermined ionic strength, pH and nucleic acid composition).

The stringent condition may be a condition which is used for hybridization between polynucleotides in well known hybridization methods in the art between polynucleotides such as PCR, microarray, Southern blotting and the like.

Specifically, the stringent condition may include a Na ion concentration (or other salt) of lower than about 1.5 M, more specifically 0.01 to 1.0 M at pH 7.0 to 9.0 and at least about 30° C. For example, the stringent condition in microarray includes hybridization in 50% formamide, 1 M NaCl and 1% SDS at 37° C. and washing in 0.1×SSC at 60 to 65° C.

The stringent condition in PCR may include pH 7.0 to 9.0, 0.01 to 0.1 M of Tris HCl, a K ion concentration (or other salt) of 0.05 to 0.15 M with at least about 55° C.

The sequence of the nucleic acid probe for detecting human Tfh cells can be appropriately selected by a person skilled in the art based on the common technical knowledge in the art and the base sequence of the present polynucleotide marker, so that the probe can specifically hybridize to the marker.

Such a sequence can be determined by, for example, using a conventionally available primer design software (for example, available from Primer3 (http://frodo.wi.mit.edu/cgi-bin/primer3/primer3.cgi)) or DNASISPro (Hitachi Software Engineering Co., Ltd.).

The nucleic acid probe for detecting human Tfh cells can be prepared by well known polynucleotide synthesis methods in the art.

The nucleic acid probe for detecting human Tfh cells may be labeled with a labeling substance which is conventionally used in the art. By using the labeled probe, the present polynucleotide marker, and thus human Tfh cells can be conveniently detected.

The labeling substance may be a labeling substance conventionally used in the art including a radioactive isotope such as ³²P, a fluorescent material such as fluorescein, an enzyme such as alkaline phosphatase or horse radish peroxidase, biotin or the like.

Human Tfh cells can be specifically detected by using the nucleic acid probe for detecting human Tfh cells alone or two or more probes in combination. For example, one or more probes may be immobilized on a substrate using the well known method in the art to prepare a DNA chip or microarray in order to detect the polynucleotide marker(s) for detecting human Tfh cells.

The nucleic acid probe for detecting human Tfh cells may be a primer set of two or more primers for amplifying the polynucleotide marker by PCR.

A molecule which can specifically bind to the present protein marker is useful for detection of human Tfh cells because it can be used for detecting the marker. The molecule may be any of nucleic acid aptamers such as DNA or RNA and antibodies, preferably antibodies which can specifically bind to the protein marker.

When the present protein marker is an enzyme, the marker can be detected by carrying out the reaction between the enzyme and a corresponding substrate to develop color, luminescence, fluorescence and the like.

The antibody for detecting human Tfh cells described above can be prepared by well known procedures as described below, for example. A DNA molecule encoding a protein having the amino acid sequence of the present protein marker, which is based on the base sequence of the gene as the present polynucleotide marker or the amino acid sequence of the present protein marker, is integrated into an appropriate expression vector. The obtained expression vector is introduced into an appropriate host cell and the obtained transformant is cultured to obtain the protein of interest. The obtained protein is purified and used as an immunogen to immunize an appropriate mammal such as rat or mouse optionally with an adjuvant. An antibody producing cell which produces an antibody directed to the immunogen of interest is screened from spleen cells of the immunized animal. The obtained antibody producing cell is fused with a myeloma cell to obtain a hybridoma of which screening gives an antibody producing hybridoma which produces an antibody specifically binding to the protein encoded by the gene. The antibody of interest can be obtained by culturing the obtained antibody producing hybridoma.

The nucleic acid aptamer which can be used for detecting human Tfh cells can be prepared according to well known procedures in the art as described below, for example. A nucleic acid library containing base sequences of random nucleic acid molecules is prepared according to the well known technique, which is then subjected to a technique such as systematic evolution of ligands by exponential enrichment (SELEX) and the like to select a nucleic acid aptamer specifically binding to the protein of interest (present protein marker).

The molecule which can specifically bind to the protein marker for detecting human Tfh cells may be labeled with a labeling substance which is conventionally used in the art. For example, by using the labeled antibody for detecting human Tfh cells, the protein marker for detecting human Tfh cells, and thus human Tfh cells can be conveniently detected.

The labeling substance may be a labeling substance conventionally used in the art including a radioactive isotope such as ³²P, a fluorescent material such as fluorescein, an enzyme such as alkaline phosphatase or horse radish peroxidase, biotin or the like.

The present polynucleotide marker and protein marker may be used in combination with a molecule which is known in the art that it is specifically expressed in Tfh cells, in order to detect Tfh cells. Such a known molecule may include, for example, CXCR5, IL-21 and the like.

Accordingly, in an embodiment of the present invention, a combination of the polynucleotide marker for CD9 and a polynucleotide having the base sequence of the gene encoding CXCR5 or a variant or fragment thereof can be used as the polynucleotide marker for detecting human Tfh cells. In another embodiment of the present invention, a combination of the protein marker for CD9 and a protein having the amino acid sequence of CXCR5 or a functionally equivalent variant or fragment thereof can be used as the protein marker for detecting human Tfh cells.

The above combinations allow detection of Tfh cells with higher accuracy than detection thereof with a sole molecule which is known to be specifically expressed in Tfh cells.

The present invention also encompasses a method for detecting human Tfh cells (hereinafter also referred to as the present method) by detecting at least one polynucleotide marker for detecting human Tfh cells or protein marker for detecting human Tfh cells according to the present invention in a sample containing cells.

In an embodiment of the present method, it is preferable to detect human Tfh cells by detecting the polynucleotide marker or protein marker for CD9.

In another embodiment of the present method, it is preferable to detect more than one polynucleotide marker or protein marker for detecting human Tfh cells in order to improve the detection sensitivity.

In the present method, human Tfh cells in a sample may be detected by detecting a combination of the present polynucleotide marker or protein marker and a molecule which is known to be specifically expressed in Tfh cells as the marker for detecting human Tfh cells. Such a combination is preferably a combination of CD9 and CXCR5 described above.

In the present method, a sample containing cells may include a biological sample obtained from human and a sample containing cultured cells. The biological sample may include blood, tissue, synovial fluid, cerebrospinal fluid, pleural fluid, ascites and the like.

An embodiment of the method for detecting the presence of the present polynucleotide marker is described hereinbelow.

A sample containing cells is processed by the well known method in the art which uses phenol extraction and ethanol precipitation or a commercially available DNA extraction kit to extract nucleic acid molecules (DNAs or RNAs).

The obtained nucleic acid sample is detected for the presence of the present polynucleotide marker. This detection is carried out preferably with the nucleic acid probe for detecting human Tfh cells described above.

The present polynucleotide marker can be detected by the well known method in the art including nucleic acid amplification methods such as PCR, RT-PCR, real time PCR, LAMP (Loop-mediated isothermal amplification) and the like; hybridization methods such as Southern hybridization, Northern hybridization, FISH (fluorescence in situ hybridization) and the like; and DNA chip and microarray methods. Such a method is carried out under the stringent condition described above and hybridization of the nucleic acid probe for detecting human Tfh cells can be detected by detecting, for example, the labeling substance described above to detect the presence of the present polynucleotide marker.

An embodiment of the method for detecting the protein marker for detecting human Tfh cells is described hereinbelow.

When, for example, the protein marker to be detected is an intracellular protein, protein is extracted from cells by the well known method in the art. Protein can be extracted from cells by the well known method in the art such as ultrasonic cell homogenization, solubilization of cells with a cell solubilizing solution and the like. The present protein marker in the protein sample can be detected with the molecule which specifically binds to the protein marker. Specifically, the protein marker can be detected by the well known method in the art such as ELISA, Western blotting and the like. In this detection, it is preferable that the antibody for detecting human Tfh cells described above is used as the molecule which specifically binds to the present protein marker.

When, for example, the present protein marker to be detected is a secretory protein, the protein marker secreted in a sample containing cells can be detected with the molecule which specifically binds to the protein marker.

Alternatively, after collecting cells (lymphocytes) from a sample containing cells and stimulating the cells with an anti-CD3 antibody, anti-CD28 antibody, concanavalin A, phytohemagglutinin (PHA), phorbol myristate acetate (PMA), ionomycin and the like, the protein marker secreted from the cells can be detected with the molecule which specifically binds to the protein marker.

Specifically, the present protein marker can be detected by the well known method in the art such as ELISA, Western blotting and the like. In this detection, it is preferable that the antibody for detecting human Tfh cells described above is used as the molecule which specifically binds to the present protein marker.

When, for example, the present protein marker to be detected is a protein existing on cell surface (cell membrane), the protein marker existing on the cell surface in a sample containing cells can be detected with the molecule which specifically binds to the protein marker.

Alternatively, after collecting a membrane fraction of cells from a sample containing cells, the present protein marker in the obtained membrane fraction can be detected with the molecule which specifically binds to the protein marker. Specifically, the protein marker can be detected by the well known method in the art such as ELISA, Western blotting, a method based on flow cytometry (FCM) and the like.

In this detection, it is preferable that the antibody for detecting human Tfh cells described above is used as the molecule which specifically binds to the present protein marker.

When, for example, the present protein marker is detected by FCM, the following procedures may be carried out.

A sample containing cells is first contacted with the antibody for detecting human Tfh cells labeled with an appropriate labeling substance. When exist, human Tfh cells bind to the labeled antibody on the cell surface. By passing the sample containing cells bound to the labeling substance through a flow cytometer, human Tfh cells can be detected. Optionally, the human Tfh cells bound to the labeling substance can be sorted and fractionated with a cell sorter.

The method based on FCM per se is well known to a person skilled in the art and he/she can appropriately choose the reaction conditions.

As described above, human Tfh cells can be specifically detected by detecting at least one polynucleotide marker or protein marker for detecting human Tfh cells according to the present embodiment. Thus, for example, detection of the above marker in a sample containing cells such as tissue collected from a subject allows specific detection of Tfh cells in the sample. By using the above marker, Tfh cells can be isolated from a sample containing various cells.

It is further possible that the marker for detecting human Tfh cells may be used for diagnoses and/or examinations of patients for diseases which Tfh cells are believed to be involved such as autoimmune diseases including SLE, Sjogren's syndrome and the like. Thus, the present invention also encompasses a marker for diagnosis and for examination of a patient for a disease which Tfh cells are believed to be involved which is the polynucleotide marker or protein marker for detecting human Tfh cells and the use thereof. The present invention also encompasses a method for diagnosing and/or examining a subject for a disease which Tfh cells are believed to be involved by using the marker.

### EXAMPLES

The present invention is described in more detail by way of Examples which do not limit the present invention.

### Example 1

### Analysis of Highly Expressed Genes in Human Peripheral Blood-Derived Cultured Tfh Cells

1. Isolation of NaïVe CD4 Positive T Cells from Human Peripheral Blood

Buffy coats obtained from peripheral blood of healthy adults were layered on Ficoll-paque plus solution (GE Healthcare Biosciences) and centrifuged to obtain the monocyte fraction. The CD4 positive cells were partially purified from the fraction by using magnetic beads (Miltenyi Biotec) coupled with an anti-CD4 antibody.

The thus obtained CD4 positive cells were stained with fluorescence-labeled antibodies shown in Table 1 prior to isolate CD4⁺CD25⁻CD45RA⁺CD45RO⁻ naïve CD4 positive T cells (hereinafter also referred to as “naïve CD4 positive T cells”) with a cell sorter (FACS Aria: Becton Dickinson).

TABLE 1

| | Fluorescence labeling | | |
|---|---|---|---|
| Antigen | substance | Clone | Manufacturer |
| CD4 | FITC | OKT4 | BioLegend |
| CD25 | PE-Cy7 | BC96 | eBioscience |
| CD45RO | PE | UCHL1 | BioLegend |
| CD45RA | APC | HI100 | BioLegend |

2. Differentiation Culture of Tfh and Th9 Cells from NaïVe CD4 Positive T Cells

The residual adult peripheral blood-derived naïve CD4 positive T cells obtained in the above step 1. were seeded in a 96-well plate at a density of 1.0×10⁵ cells/0.3 ml/well. The medium used was the Yssel medium.

In order to allow activation and proliferation of the above cells, antibody beads were added to each well at 0.5×10⁵. The cells were added with cytokines and neutralizing antibodies suitable for respective differentiation culture of Tfh cells and Th9 cells (see Table 2) and cultured in an incubator at 37° C. and 5% CO₂. The concentration of all cytokines used is 10 ng/ml and the concentration of all neutralizing antibodies used is 2.5 μg/ml. The cytokines and neutralizing antibodies used were purchased from R&D Systems and Biolegend.

TABLE 2

| Cell | Cytokine | Neutralizing antibody (clone) |
|---|---|---|
| Th9 | TGF-β1, IL-4, IL-2 | Anti IFN-γ antibody (R4-6A2) |
| Tfh | TGF-β1, IL-12, IL-2 | Anti IL-4 antibody (MP4-25D2), |
| | | Anti IFN-γ antibody (R4-6A2), |
| | | Anti IL-6 antibody (MQ2-13A5) |

Cytokines are from R&D Systems and all neutralizing antibodies are from Biolegend.

After three days from the initiation of culture, the cells were diluted by three times with the medium containing the above cytokines and antibodies and cultured for further 4 days (7 days in total) to obtain Tfh cells and Th9 cells (Tfh cells obtained in this step are hereinafter referred to as “Tfh cells (1)”).

The obtained Tfh cells (1) and Th9 cells were divided into two portions, each one of which was washed with the Yssel medium and PBS followed by collection of the cells by centrifugation and freezing at −80° C. for storage of the cells until the next RNA extraction step. These cells are designated as Tfh cells (1) and Th9 cells “without activation stimulation”, respectively. The other portion of the cells were respectively added with the antibody beads described above and cultured for further 3 hours to re-activate the cells. The cells were then recovered by centrifugation and frozen for storage at −80° C. in a similar manner. These cells were designated as Tfh cells (1) and Th9 cells “with activation stimulation”, respectively.

3. Isolation of Tfh Cells (2) from Human Peripheral Blood

Buffy coats obtained from peripheral blood of healthy adults were layered on Ficoll-paque plus solution (GE Healthcare Biosciences) and centrifuged to obtain the monocyte fraction. The CD4 positive cells were partially purified from the fraction by using magnetic beads (Miltenyi Biotec) coupled with an anti-CD4 antibody. The thus obtained CD4 positive cells were stained with fluorescence-labeled antibodies shown in Table 3 prior to isolate Tfh cells with a cell sorter (FACS Aria: Becton Dickinson) (the Tfh cells obtained in this step are hereinafter referred to as “Tfh cells (2)”). The settings of gating for isolation are shown in Table 4.

TABLE 3

| | Fluorescence labeling | | |
|---|---|---|---|
| Antigen | substance | Clone | Manufacturer |
| CD4 | APC-Cy7 | RPA-T4 | BD Biosciences |
| CD25 | PE-Cy7 | BC96 | eBioscience |
| CXCR3 | Alexa fluor ™ 488 | 1C6/CXCR3 | BD Biosciences |
| CXCR5 | PerCP-Cy5.5 | TG2/CXCR5 | Bio Legend |
| CCR4 | APC | TG6/CCR4 | Bio Legend |
| CCR6 | PE | 11A9 | BD Biosciences |

TABLE 4

| Cell | Gating |
|---|---|
| Tfh(2) | CD4^{high} CD25^{low-negative} CXCR3⁻ CXCR5⁺ CCR4⁻ CCR6⁻ |

### 4. Culture of Tfh Cells (2)

The adult peripheral blood-derived Tfh cells (2) obtained in the above step 3. were seeded in a 96-well plate at a density of 1.0×10⁵ cells/0.3 ml/well. The medium used was the Yssel medium.

In order to activate and proliferate the above cells, magnetic beads coated with an anti-CD2/3/28 antibody (Miltenyi Biotec) (hereinafter also referred to as “antibody beads”) were added to each well at 0.5×10⁵. The cells were added with cytokines and neutralizing antibodies suitable for differentiation culture of the Tfh cells (2) (Table 5) and cultured in an incubator at 37° C. and 5% CO₂.

TABLE 5

| Cell | Cytokine | Neutralizing antibody |
|---|---|---|
| Tfh(2) | TGF-β1, IL-12, IL-2 | Anti IL-4 antibody (MP4-25D2), |
| | | Anti IL-6 antibody (MQ2-13A5), |
| | | AntiIFN-γ(R4-6A2) |

The concentration of all cytokines used is 10 ng/ml and the concentration of all neutralizing antibodies used is 2.5 μg/ml. The cytokines and neutralizing antibodies used were purchased from R&D Systems and Biolegend. After three days from the initiation of culture, the cells were diluted by three times with the medium containing the above cytokines and antibodies and cultured for further 4 days (7 days in total) to obtain Tfh cells (2).

The obtained Tfh cells (2) were divided into two portions, one of which was washed with the Yssel medium and PBS followed by collection of the cells by centrifugation and freezing at −80° C. for storage of the cells until the next RNA extraction step. These cells are designated as Tfh cells (2) “without activation stimulation”. The other portion of the cells was added with the antibody beads described above and cultured for further 3 hours to re-activate the cells. The cells were then recovered by centrifugation and frozen for storage at −80° C. in a similar manner. These cells were designated as Tfh cells (2) “with activation stimulation”.

5. Isolation of Th1, Th2, Treg, Th17 and Th22 Cells from Human Peripheral Blood
(1) Isolation of Th1, Th2, Th17 and Th22 Cells from Human Peripheral Blood

Buffy coats obtained from peripheral blood of healthy adults were layered on Ficoll-paque plus solution (GE Healthcare Biosciences) and centrifuged to obtain the monocyte fraction. The CD4 positive cells were partially purified from the fraction by using magnetic beads (Miltenyi Biotec) coupled with an anti-CD4 antibody. The thus obtained CD4 positive cells were stained with fluorescence-labeled antibodies shown in Table 6 prior to isolate Th1, Th2, Th17 and Th22 cells respectively with a cell sorter (FACS Aria: Becton Dickinson). The settings of gating for isolation are shown in Table 7.

TABLE 6

| | Fluorescence labeling | | |
|---|---|---|---|
| Antigen | substance | Clone | Manufacturer |
| CD4 | APC-Cy7 | RPA-T4 | BD Biosciences |
| CD25 | PE-Cy7 | BC96 | eBioscience |
| CXCR3 | Alexa fluor ™ 488 | 1C6/CXCR3 | BD Biosciences |
| CCR4 | APC | FAB1567A | R&D systems |
| CCR6 | PE | 11A9 | BD Biosciences |
| CD45RA | APC | HI100 | BioLegend |
| CCR10 | PE | 6588-5 | BioLegend |

TABLE 7

| Cell | Gating |
|---|---|
| Th1 | CD4^{high} CD25^{low-negative} CXCR3⁺ CCR6⁻ CCR4⁻ |
| Th2 | CD4^{high} CD25^{low-negative} CXCR3⁻ CCR6⁻ CCR4⁺ |
| Th17 | CD4^{high} CD25^{low-negative} CXCR3⁻ CCR6⁺ CCR4⁺ |
| Th22 | CD4^{high} CD25^{low-negative} CD45RA⁻ CXCR3⁻ CCR10⁺ |

The details for the above procedures for sorting can be found in Acosta-Rodriguez E V. et al. (“Surface phenotype and antigenic specificity of human interleukin 17-producing T helper memory cells.”, Nat Immunol., vol. 8, p. 639-646 (2007)) and Trifari S. et al. (“Identification of a human helper T cell population that has abundant production of interleukin 22 and is distinct from TH-17, TH1 and TH2 cells.”, Nat Immunol., vol. 10, p. 864-871 (2009)).

(2) Isolation of Treg Cells from Human Peripheral Blood

The CD4 positive cells obtained in the similar manner as the above step (1) were stained with fluorescence-labeled antibodies shown in Table 8 prior to purify Treg cells which were CD4^{high} CD25^{high} CD127^{internal-negative} cells on the cell sorter described above.

TABLE 8

| | Fluorescence labeling | | |
|---|---|---|---|
| Antigen | substance | Clone | Manufacturer |
| CD4 | FITC | OKT4 | eBioscience |
| CD25 | PE-Cy7 | BC96 | eBioscience |
| CD45RO | PE | UCHL1 | BioLegend |
| CD127 | Alexa fluor ™ 647 | HIL-7R-M21 | BD Biosciences |

The details for the above procedures for sorting can be found in Weihong Liu. et al. (“CD127 expression inversely correlates with FoxP3 and suppressive function of human CD4+ Treg cells.”, J Exp Med., vol. 203, p. 1701-1711 (2006)).

### 6. Culture of Th1, Th2, Treg, Th17 and Th22 Cells

### (1) Culture of Th1, Th2, Th17 and Th22 Cells

The adult peripheral blood-derived Th1, Th2, Th17 and Th22 cells obtained in the above step 5.(1) were respectively seeded in a 96-well plate at a density of 1.5×10⁵ cells/0.3 ml/well. The medium used was the Yssel medium (IMDM, 1% type AB human serum, 0.25% BSA, 1.8 mg/l 2-aminomethanol, 40 mg/l transferrin, 5 mg/l insulin, 2 mg/l linoleic acid, 2 mg/l oleic acid, 2 mg/l palmitic acid and 1% penicillin/streptomycin). In order to activate and proliferate the above cells, magnetic beads coated with an anti-CD2/3/28 antibody (Miltenyi Biotec) (hereinafter also referred to as “antibody beads”) were added to each well at 0.75×10⁵. The cells were added with cytokines and neutralizing antibodies suitable for differentiation culture of Th1, Th2, Th17 and Th22 cells, respectively, and cultured in an incubator at 37° C. and 5% CO₂. The cytokines and neutralizing antibodies used are shown in Table 9.

TABLE 9

| Cell | Cytokine | Neutralizing antibody (clone) |
|---|---|---|
| Th1 | 1L-12, IL-2 | Anti IL-4 antibody (MP4-25D2) |
| Th2 | IL-4, IL-2 | Anti IFN-γantibody (R4-6A2) |
| Th17 | TGF-β1, IL-6, IL-23, | Anti IL-4 antibody(MP4-25D2), |
| | IL-21, IL-1β, TNFα, IL-2 | Anti IFN-γantibody(R4-6A2) |
| Th22 | IL-6, TNFα, IL-2 | Anti IL-4 antibody (MP4-25D2), |
| | | Anti IFN-γantibody (R4-6A2), |
| | | Anti TGF-βantibody (9016) |

The concentration of cytokines was 50 ng/ml for IL-6 and 10 ng/ml for those other than IL-6. The concentration of the antibodies was 10 μg/ml for the anti-IFN-γ antibody and 2.5 μg/ml for the anti-IL-4 and anti-TGF-β antibodies. The cytokines and neutralizing antibodies were purchased from R&D systems and eBioscience, respectively. After three days from the initiation of culture, the cells were diluted by three times with the medium containing the above cytokines and antibodies and cultured for further 7 days (10 days in total) to obtain Th1, Th2, Th17 and Th22 cells. The obtained cells were respectively divided into two portions, each one of which was washed with the Yssel medium and PBS followed by collection of the cells by centrifugation and freezing at −80° C. for storage of the cells until the next RNA extraction step. These cells are designated as Th1, Th2, Th17 and Th22 cells “without activation stimulation”. The other portion of the cells was added with the antibody beads described above and cultured for further 3 hours to re-activate the cells. The cells were then recovered by centrifugation and frozen for storage at −80° C. in a similar manner. These cells were respectively designated as Th1, Th2, Th17 and Th22 cells “with activation stimulation”.

### (2) Culture of Treg Cells

The Treg cells obtained in the above step 5.(2) were cultured in the Yssel medium as described in the above step 6.(1) and activated with the antibody beads. To the medium were added cytokines IL-2 and TGF-β1 (R&D systems) and neutralizing antibodies anti-IFN-γ and anti-IL-4 antibodies (eBioscience) and anti-IL-6 antibody (BD bioscience). The concentration of these cytokines and neutralizing antibodies was 10 ng/ml and 5 μg/ml, respectively. After three days from the initiation of culture, the cells were added with the cytokines and neutralizing antibodies at the same amounts as those at the initiation of culture. After additional three days of culture, the cells were divided into two portions, each of which was added with or without the antibody beads and cultured for further 3 days to obtain Treg cells “with activation stimulation” or “without activation stimulation”. The respective Treg cells were collected by centrifugation and frozen at −80° C. for storage until the next RNA extraction step.

### 7. Verification of Purity of Differentiation Cultured Cells by Flow Cytometry

To the respective cell suspensions containing the cells after differentiation culture (respectively containing 2.5×10⁵ cells) were added phorbol myristate acetate (PMA: 50 ng/ml) and ionomycin (1 μM) to stimulate the cells. After 4 hours, the cells were added with brefeldin A (10 μg/ml) and cultured for additional 2 hours. The cells were washed with phosphate buffered saline (PBS) and fixed with 4% paraformaldehyde. After fixation, the cells were treated with a saponin buffer (0.5% saponin, 0.5% bovine serum albumin (BSA), 1 mM sodium azide (in PBS)) in order to increase permeability of the cell membrane. The cells were allowed to react with a fluorescence (FITC, PE, APC, PerCP-Cy5.5, Alexa647 etc.)-labeled anti-IFN-γ, anti-IL-4, anti-IL-17, anti-Foxp3, anti-IL-9, anti-IL-22 or anti-IL-21 antibody. After the reaction, the cells were washed with the saponin buffer and then with a 0.5% BSA-containing PBS and analyzed on FACS Canto II (Becton Dickinson) to confirm the purity of the cells. The items used for confirmation of purity were respectively as follows: Th1 cells: IFN-γ, Th2 cells: IL-4, Th17 cells: IL-17A, Treg cells: Foxp3 (transcription factor), Th9 cells: IL-9, Th22 cells: IL-22 and Tfh cells (1) and (2): IL-21.

### 8. Extraction of Total RNA

In order to extract total RNA from the cells obtained in the above steps 1., 2., 4. and 6., the RNeasy Plus Mini kit and the RNeasy micro kit (QIAGEN) were respectively used. The specific procedures followed the instructions attached to each kit.

### 9. Microarray Expression Assay

Total RNA (10 to 100 ng) extracted from the cells in the step 7. was reverse-transcribed to cDNA and further transcribed to biotinylated cRNA with Two-Cycle Target Labeling and Control Reagents (Affymetrix). The amplified biotinylated cRNA (20 μg) was then fragmented. The specific procedures followed the instructions attached to the kit.

The biotinylated cRNA (15 μg) from the respective cells obtained as above was used as a sample and added to the GeneChip Human Genome U-133 Plus 2.0 Array (Affymetrix), which was then transferred to the GeneChip Hybridization Oven 640 (Affymetrix) in order to carry out hybridization at 45° C. and 60 rpm for 16 hours. After hybridization, the microarray was washed and fluorescence-labeled on the GeneChip Fluidic Station 450 (Affymetrix) and scanned on the GeneChip Scanner 3000 7G (Affymetrix) to acquire fluorescence intensity data.

### 10. Selection of Genes Specifically Expressed in Human Th22 Cells

The fluorescence intensity data obtained in the above step 8. was standardized with MASS algorithm on the expression assay software GeneSpring GX Ver.11 (Agilent Technologies) in order to obtain relative fluorescence intensity of genes in the cells. The relative fluorescence intensity corresponds to the amount of gene expression in the cells. The obtained relative fluorescence intensity of the genes in Tfh cells (1) was compared with the relative fluorescence intensity in naïve CD4 positive T cells, Th1, Th2, Treg, Th17, Th9 and Th22 cells. The genes which have the relative fluorescence intensity in Tfh cells (1) three times or more than the relative fluorescence intensity in all naïve CD4 positive T cells, Th1, Th2, Treg, Th17, Th9 and Th22 cells and which are expressed significantly (the genes which showed “p value<0.05” in ANOVA test for the relative fluorescence intensity between 8 groups of naïve CD4 positive T cells, Th1, Th2, Treg, Th17, Th9 and Th22 cells and Tfh cells (1)) were identified as the genes specifically expressed in Tfh cells (1).

In the similar manner, the genes which have the relative fluorescence intensity in Tfh cells (2) three times or more than the relative fluorescence intensity in all naïve CD4 positive T cells, Th1, Th2, Treg, Th17, Th9 and Th22 cells and which are expressed significantly (the genes which showed “p value<0.05” in ANOVA test for the relative fluorescence intensity between 8 groups of naïve CD4 positive T cells, Th1, Th2, Treg, Th17, Th9 and Th22 cells and Tfh cells (2)) were identified as the genes specifically expressed in Tfh cells (2). The number of samples used in this selection step is shown in Table 10.

TABLE 10

| | Naive CD4 | | | | |
|---|---|---|---|---|---|
| | positive T cell | Th1 | Th2 | Th17 | Treg |
| w/o activation stimulation | 4 | 5 | 5 | 5 | 4 |
| w/ activation stimulation | 3 | 5 | 5 | 5 | 3 |

| | Th22 | Th9 | Tfh (1) | Tfh (2) |
|---|---|---|---|---|
| w/o activation stimulation | 5 | 3 | 3 | 3 |
| w/ activation stimulation | 5 | 3 | 3 | 3 |

The genes which are specifically expressed in Tfh cells (1) and Tfh cells (2) under the conditions “with activation stimulation” and “without activation stimulation” and the ratio of expression amount of the genes between the cells are shown in Tables 11 to 14. The genes shown in Tables 11 and 12 are the genes which are specifically expressed in Tfh cells (1) “without activation stimulation” and “with activation stimulation”, respectively. The genes shown in Tables 13 and 14 are the genes which are specifically expressed in Tfh cells (2) “without activation stimulation” and “with activation stimulation”, respectively. “Localization” in the Tables means intracellular localization of proteins encoded by the genes.

TABLE 11

| | | | Relative | | | | | | | |
|---|---|---|---|---|---|---|---|---|---|---|
| | | | fluorescence | Expression ratio | | | | | | |
| | Gene | Probe Set | Intensity | Tfh (1)/ | Tfh (1)/ | Tfh (1)/ | | | | |
| Localization | symbol | ID | Tfh (1) | Naïve | Th1 | Th17 | Tfh (1)/Th2 | Tfh (1)/Th22 | Tfh (1)/Th9 | Tfh (1)/Treg |
| Membrane | ART3 | 210147_at | 399.0 | 20.6 | 6.6 | 11.1 | 20.4 | 16.9 | 6.3 | 7.7 |
| | CD28 | 211861_x_at | 545.4 | 6.8 | 7.7 | 10.9 | 35.2 | 10.8 | 4.0 | 4.4 |
| | CD9 | 201005_at | 6965.1 | 26.3 | 274.0 | 99.2 | 553.6 | 23.6 | 10.1 | 6.0 |
| | CDH3 | 203256_at | 194.6 | 23.5 | 19.4 | 22.3 | 22.3 | 30.4 | 11.8 | 30.0 |
| | ELOVL7 | 227180_at | 673.8 | 100.6 | 27.0 | 98.0 | 36.6 | 127.9 | 111.5 | 14.4 |
| | EMP2 | 225079_at | 108.0 | 7.7 | 9.9 | 12.8 | 9.9 | 12.6 | 6.5 | 7.4 |
| | FAM26F | 229543_at | 397.7 | 7.0 | 3.2 | 6.8 | 14.9 | 12.4 | 5.5 | 24.6 |
| | FUT7 | 210506_at | 3252.5 | 97.3 | 19.0 | 14.5 | 31.1 | 12.9 | 3.3 | 16.3 |
| | GABBR1, | 205890_s_at | 1853.0 | 106.1 | 35.9 | 147.9 | 449.4 | 118.9 | 221.8 | 289.5 |
| | UBD | | | | | | | | | |
| | GPC4 | 204984_at | 113.6 | 5.4 | 6.7 | 6.9 | 10.3 | 6.8 | 24.7 | 11.8 |
| | GPR84 | 223767_at | 310.0 | 70.8 | 16.1 | 25.4 | 5.1 | 12.9 | 3.8 | 5.1 |
| | IL12RB2 | 206999_at | 4426.6 | 45.7 | 3.1 | 53.8 | 129.3 | 49.4 | 265.1 | 6.9 |
| | IL23R | 1552912_a_at | 444.6 | 49.6 | 30.3 | 3.7 | 56.2 | 36.4 | 62.0 | 15.1 |
| | | 1561853_a_at | 194.5 | 21.6 | 31.0 | 5.3 | 27.2 | 17.5 | 36.2 | 13.9 |
| | KCNS3 | 205968_at | 158.1 | 14.4 | 10.5 | 10.7 | 8.3 | 18.4 | 5.5 | 18.6 |
| | KISS1R | 242517_at | 1441.3 | 7.9 | 11.5 | 4.5 | 5.9 | 8.5 | 5.1 | 12.9 |
| | LHFP | 218656_s_at | 369.5 | 12.9 | 12.4 | 15.3 | 4.5 | 7.3 | 6.8 | 4.7 |
| | LST1 | 214181_x_at | 675.6 | 3.3 | 25.7 | 28.7 | 25.2 | 9.7 | 3.8 | 6.8 |
| | PLSCR1 | 202430_s_at | 4742.1 | 4.6 | 3.6 | 3.5 | 18.2 | 10.7 | 4.1 | 3.1 |
| | | 202446_s_at | 10186.2 | 7.1 | 3.1 | 3.4 | 18.3 | 6.9 | 3.2 | 3.7 |
| | PPP2CB | 201374_x_at | 53.0 | 7.8 | 20.7 | 17.3 | 17.5 | 7.9 | 5.0 | 13.6 |
| | RHOU | 223169_s_at | 987.8 | 8.0 | 13.9 | 7.4 | 26.5 | 13.2 | 3.5 | 5.2 |
| | SLC35E4 | 1568623_a_at | 821.6 | 59.2 | 7.9 | 6.1 | 9.8 | 6.5 | 4.9 | 5.9 |
| | TAC1 | 206552_s_at | 52.5 | 4.8 | 21.0 | 26.7 | 13.0 | 15.5 | 6.3 | 3.2 |
| | THBS1 | 201109_s_at | 449.2 | 77.6 | 97.5 | 37.0 | 110.8 | 50.2 | 28.6 | 10.6 |
| | | 201110_s_at | 1306.3 | 224.7 | 367.2 | 56.0 | 336.6 | 51.2 | 21.5 | 12.6 |
| | TM4SF1 | 209386_at | 250.1 | 9.1 | 7.2 | 12.7 | 7.3 | 4.2 | 4.1 | 5.1 |
| | | 209387_s_at | 139.5 | 15.6 | 14.5 | 47.8 | 30.9 | 14.6 | 6.5 | 21.5 |
| | TMEM213 | 240183_at | 696.7 | 103.8 | 7.0 | 4.2 | 137.4 | 58.7 | 253.7 | 91.1 |
| Extracellular/ | APOD | 201525_at | 899.0 | 24.6 | 15.1 | 19.9 | 16.7 | 29.2 | 10.8 | 10.4 |
| secretory | CSF2 | 210229_s_at | 1379.0 | 89.9 | 14.3 | 101.6 | 62.0 | 106.3 | 7.0 | 76.0 |
| | CXCL10 | 204533_at | 9642.6 | 130.4 | 13.8 | 19.7 | 6.0 | 4.7 | 120.6 | 39.5 |
| | CXCL11 | 210163_at | 1113.9 | 73.9 | 21.6 | 12.3 | 26.5 | 40.2 | 80.6 | 30.9 |
| | | 211122_s_at | 1540.6 | 114.9 | 69.6 | 29.8 | 146.7 | 110.9 | 50.7 | 69.1 |
| | CXCL13 | 205242_at | 1582.5 | 303.8 | 454.6 | 7.9 | 158.5 | 281.7 | 29.8 | 36.2 |
| | CXCL9 | 203915_at | 420.4 | 4.6 | 3.0 | 8.7 | 7.8 | 8.7 | 7.8 | 3.9 |
| | FGF18 | 206987_x_at | 47.6 | 5.4 | 5.1 | 8.2 | 6.0 | 12.5 | 9.4 | 4.7 |
| | IL2 | 207849_at | 1511.5 | 111.3 | 731.4 | 285.5 | 528.4 | 116.3 | 58.1 | 157.6 |
| | IL21 | 221271_at | 968.9 | 28.0 | 9.1 | 57.5 | 24.5 | 24.2 | 63.5 | 36.6 |
| | IL8 | 211506_s_at | 294.2 | 22.5 | 6.8 | 5.2 | 40.3 | 63.2 | 10.1 | 36.3 |
| | MDK | 209035_at | 199.7 | 9.5 | 16.2 | 7.9 | 19.0 | 13.0 | 4.8 | 8.5 |
| | PTHLH | 206300_s_at | 409.7 | 27.0 | 16.7 | 7.6 | 30.3 | 18.0 | 27.4 | 4.7 |
| | | 210355_at | 328.3 | 12.2 | 13.6 | 5.9 | 42.7 | 26.1 | 8.4 | 5.7 |
| | SERPING1 | 200986_at | 133.3 | 11.3 | 18.9 | 26.3 | 14.2 | 37.4 | 20.5 | 8.5 |
| Intracellular | ANXA3 | 209369_at | 1651.7 | 30.8 | 9.9 | 133.0 | 150.9 | 84.4 | 67.2 | 19.8 |
| | BSPRY | 218792_s_at | 1143.1 | 8.9 | 3.4 | 9.2 | 9.3 | 10.8 | 15.7 | 4.3 |
| | | 222746_s_at | 1048.6 | 8.2 | 4.5 | 10.3 | 12.0 | 14.3 | 10.7 | 4.5 |
| | C10orf10 | 209183_s_at | 481.1 | 11.6 | 14.6 | 33.8 | 44.7 | 33.9 | 11.0 | 5.7 |
| | ELL | 1565254_s_at | 67.0 | 7.7 | 9.7 | 16.0 | 7.6 | 21.9 | 3.0 | 4.1 |
| | FBXO17, | 220233_at | 164.1 | 21.7 | 41.0 | 5.6 | 29.6 | 23.7 | 23.0 | 17.6 |
| | SARS2 | | | | | | | | | |
| | INSC | 237056_at | 166.6 | 41.6 | 17.0 | 58.1 | 40.3 | 21.6 | 35.5 | 10.1 |
| | MAF | 209347_s_at | 106.4 | 16.1 | 11.6 | 4.8 | 29.3 | 12.6 | 7.5 | 4.5 |
| | MAST4 | 1554652_s_at | 130.6 | 5.8 | 3.9 | 9.1 | 4.2 | 9.6 | 7.5 | 8.5 |
| | MGST1 | 1565162_s_at | 91.5 | 20.7 | 25.2 | 25.1 | 19.4 | 35.5 | 12.8 | 20.3 |
| | MYH6 | 214468_at | 235.5 | 8.7 | 5.6 | 8.5 | 8.1 | 6.6 | 30.9 | 4.2 |
| | MYH6, | 204737_s_at | 55.8 | 9.5 | 7.0 | 9.2 | 14.4 | 21.9 | 10.5 | 8.5 |
| | MYH7 | | | | | | | | | |
| | PCGF2 | 214239_x_at | 464.8 | 12.5 | 12.9 | 3.8 | 15.5 | 14.5 | 5.8 | 9.7 |
| | PLOD2 | 202619_s_at | 93.1 | 9.9 | 9.4 | 4.5 | 14.7 | 13.9 | 3.5 | 11.1 |
| | PNMA2 | 209598_at | 189.1 | 3.4 | 4.4 | 5.3 | 8.8 | 4.5 | 3.8 | 6.8 |
| | RGS20 | 210138_at | 49.6 | 9.4 | 15.6 | 10.9 | 4.2 | 9.4 | 5.0 | 5.6 |
| | S100A9 | 203535_at | 230.7 | 19.2 | 5.3 | 24.4 | 21.1 | 59.6 | 23.2 | 27.7 |
| | SOCS3 | 206359_at | 408.2 | 18.9 | 10.3 | 13.9 | 60.6 | 28.6 | 4.8 | 14.2 |
| | STAG3 | 219753_at | 1534.7 | 9.8 | 17.6 | 12.8 | 13.5 | 21.4 | 4.8 | 8.3 |
| | VPS53 | 219794_at | 71.3 | 8.6 | 14.8 | 11.7 | 8.2 | 16.6 | 4.6 | 11.1 |
| | XAF1 | 206133_at | 2239.5 | 10.9 | 7.3 | 13.9 | 22.8 | 5.4 | 4.9 | 4.0 |
| | ZNF334 | 220022_at | 47.7 | 3.5 | 6.0 | 24.2 | 7.8 | 12.5 | 3.1 | 3.4 |
| | ZNF503 | 227195_at | 164.7 | 12.3 | 12.9 | 4.2 | 7.0 | 15.5 | 4.3 | 5.0 |
| Unknown | DNAJC12 | 223721_s_at | 1136.9 | 47.9 | 102.1 | 67.4 | 206.6 | 197.8 | 5.0 | 24.9 |
| | | 223722_at | 425.1 | 15.3 | 8.3 | 3.6 | 15.6 | 25.1 | 5.8 | 4.6 |
| | GADD45B | 209305_s_at | 1109.3 | 6.3 | 3.9 | 10.9 | 14.2 | 8.2 | 5.2 | 4.1 |
| | LMO4 | 209204_at | 320.0 | 7.1 | 4.9 | 5.9 | 6.0 | 3.7 | 3.5 | 5.1 |
| | LOC400043 | 226582_at | 143.5 | 17.6 | 9.7 | 10.3 | 10.1 | 11.9 | 5.9 | 10.8 |
| | MYO1B | 212364_at | 378.0 | 3.2 | 4.8 | 31.6 | 12.0 | 16.6 | 4.6 | 12.2 |
| | NAPSB | 228055_at | 1914.1 | 73.8 | 13.5 | 13.5 | 275.7 | 420.4 | 9.1 | 14.1 |
| | | 228056_s_at | 549.0 | 149.4 | 4.6 | 4.6 | 232.9 | 206.9 | 6.6 | 15.9 |
| | NPHP1 | 238844_s_at | 54.2 | 9.6 | 10.5 | 6.0 | 7.0 | 11.8 | 3.3 | 14.2 |
| | RASSF4 | 226436_at | 163.7 | 11.7 | 4.6 | 28.9 | 19.7 | 9.9 | 15.8 | 4.7 |
| | YAP1 | 213342_at | 99.7 | 6.7 | 5.1 | 6.2 | 12.0 | 5.0 | 6.4 | 6.2 |
| | | 224894_at | 351.5 | 10.0 | 4.7 | 4.4 | 13.5 | 7.7 | 33.8 | 11.8 |
| | — | 215314_at | 656.1 | 3.1 | 3.0 | 15.8 | 4.2 | 6.6 | 3.1 | 25.3 |
| | — | 239653_at | 72.5 | 8.6 | 11.3 | 6.9 | 10.0 | 15.3 | 22.3 | 13.3 |
| | — | 242203_at | 451.2 | 22.2 | 9.8 | 14.5 | 14.9 | 44.2 | 27.3 | 22.0 |
| | — | 244434_at | 19.5 | 14.2 | 7.5 | 16.5 | 14.5 | 16.3 | 5.6 | 8.0 |
| | — | 1556773_at | 211.0 | 22.0 | 30.7 | 8.7 | 30.5 | 23.7 | 17.8 | 3.1 |
| | — | 1561654_at | 143.3 | 4.4 | 4.0 | 8.7 | 11.1 | 9.6 | 5.4 | 5.8 |

TABLE 12

| | | | Relative | | | | | | | |
|---|---|---|---|---|---|---|---|---|---|---|
| | | | fluorescence | Expression ratio | | | | | | |
| | Gene | Probe Set | Intensity | Tfh (1)/ | Tfh (1)/ | Tfh (1)/ | | | | |
| Localization | symbol | ID | Tfh (1) | Naïve | Th1 | Th17 | Tfh (1)/Th2 | Tfh (1)/Th22 | Tfh (1)/Th9 | Tfh (1)/Treg |
| Membrane | ART3 | 210147_at | 399.6 | 126.9 | 9.4 | 4.4 | 9.4 | 8.1 | 8.1 | 7.0 |
| | CD28 | 211861_x_at | 587.6 | 4.6 | 9.7 | 10.9 | 25.6 | 12.6 | 4.4 | 3.4 |
| | CD9 | 201005_at | 7075.7 | 24.5 | 437.9 | 105.5 | 377.9 | 37.0 | 9.0 | 4.1 |
| | CDH3 | 203256_at | 250.5 | 18.8 | 22.0 | 44.4 | 26.0 | 84.4 | 14.0 | 40.9 |
| | EMP2 | 225079_at | 98.5 | 12.5 | 9.2 | 11.1 | 18.5 | 5.1 | 3.5 | 7.0 |
| | FAM26F | 229543_at | 301.5 | 6.5 | 3.2 | 5.9 | 9.4 | 12.0 | 4.1 | 12.6 |
| | FCGR1B | 214511_x_at | 105.8 | 5.9 | 11.8 | 12.4 | 13.1 | 15.7 | 19.8 | 10.3 |
| | GABBR1, | 205890_s_at | 1989.3 | 171.6 | 34.8 | 226.4 | 367.8 | 106.2 | 255.3 | 90.8 |
| | UBD | | | | | | | | | |
| | GPC4 | 204984_at | 79.6 | 4.3 | 5.6 | 3.2 | 4.0 | 9.6 | 8.9 | 4.6 |
| | IL23R | 1552912_a_at | 519.3 | 70.0 | 34.3 | 4.0 | 111.0 | 13.8 | 17.5 | 10.8 |
| | | 1561853_a_at | 264.9 | 59.1 | 56.6 | 3.8 | 29.2 | 40.1 | 10.3 | 23.2 |
| | KCNS3 | 205968_at | 109.8 | 11.6 | 4.4 | 8.0 | 5.8 | 18.1 | 10.6 | 6.6 |
| | KIAA1244 | 231856_at | 45.6 | 9.5 | 13.5 | 9.9 | 10.4 | 9.7 | 3.7 | 17.7 |
| | KISS1R | 242517_at | 1236.6 | 10.5 | 20.3 | 5.9 | 12.3 | 6.3 | 5.7 | 14.9 |
| | LHFP | 218656_s_at | 532.6 | 18.3 | 16.8 | 13.1 | 4.8 | 6.7 | 5.5 | 5.8 |
| | LST1 | 211582_x_at | 709.5 | 3.4 | 25.3 | 56.0 | 24.9 | 8.8 | 4.0 | 8.2 |
| | NTRK3 | 215025_at | 81.3 | 6.0 | 12.6 | 12.1 | 13.4 | 10.1 | 3.2 | 9.3 |
| | PAWR | 204005_s_at | 157.7 | 3.5 | 14.6 | 8.6 | 23.8 | 5.7 | 4.1 | 4.9 |
| | PLSCR1 | 202430_s_at | 5299.8 | 4.3 | 5.5 | 4.6 | 38.7 | 7.9 | 3.7 | 3.9 |
| | RHOU | 223169_s_at | 597.0 | 4.5 | 10.3 | 7.4 | 24.0 | 22.8 | 3.1 | 4.1 |
| | THBS1 | 201109_s_at | 566.7 | 35.6 | 78.4 | 20.1 | 83.3 | 29.0 | 12.2 | 10.8 |
| | | 201110_s_at | 1909.5 | 56.6 | 290.8 | 35.4 | 197.4 | 80.1 | 21.8 | 10.4 |
| | TM4SF1 | 209386_at | 565.1 | 17.4 | 23.5 | 24.2 | 42.9 | 10.9 | 8.4 | 14.1 |
| | | 209387_s_at | 292.4 | 31.9 | 14.9 | 91.2 | 31.2 | 16.4 | 7.5 | 24.8 |
| | | 215034_s_at | 274.4 | 43.3 | 22.0 | 68.4 | 15.2 | 40.7 | 11.1 | 49.0 |
| | TMCC2 | 213096_at | 646.8 | 4.6 | 4.2 | 7.5 | 8.8 | 11.1 | 3.2 | 8.9 |
| | TMEM213 | 239593_at | 667.1 | 14.1 | 7.5 | 4.3 | 30.0 | 33.3 | 34.7 | 22.1 |
| | | 240183_at | 384.9 | 29.7 | 16.2 | 9.2 | 58.4 | 58.4 | 111.1 | 80.0 |
| Extracellular/ | APOD | 201525_at | 833.8 | 21.9 | 14.0 | 16.8 | 15.5 | 16.5 | 11.3 | 8.9 |
| secretory | CCL2 | 216598_s_at | 153.3 | 20.3 | 30.1 | 32.5 | 13.5 | 31.8 | 30.6 | 29.2 |
| | COL6A2 | 209156_s_at | 186.6 | 3.9 | 21.7 | 34.9 | 38.5 | 39.8 | 3.1 | 6.1 |
| | CXCL11 | 210163_at | 1427.6 | 31.1 | 18.7 | 11.4 | 33.2 | 17.9 | 55.5 | 29.2 |
| | | 211122_s_at | 2001.1 | 66.6 | 68.7 | 24.9 | 120.5 | 32.3 | 109.0 | 103.2 |
| | CXCL13 | 205242_at | 1602.7 | 295.5 | 365.3 | 7.7 | 310.7 | 298.1 | 9.4 | 36.8 |
| | FGF18 | 211029_x_at | 80.8 | 5.6 | 10.7 | 8.5 | 8.0 | 13.1 | 3.0 | 13.2 |
| | FGF2 | 204422_s_at | 239.9 | 4.9 | 4.4 | 7.6 | 4.5 | 12.6 | 6.2 | 11.2 |
| | IFNB1 | 208173_at | 78.5 | 12.3 | 13.6 | 7.5 | 18.7 | 14.4 | 16.2 | 7.5 |
| | IL21 | 221271_at | 9455.7 | 44.0 | 8.8 | 75.9 | 50.3 | 13.8 | 21.4 | 128.1 |
| | IL8 | 211506_s_at | 7252.7 | 50.1 | 9.8 | 5.7 | 23.4 | 47.9 | 3.3 | 88.3 |
| | MDK | 209035_at | 194.8 | 18.0 | 15.9 | 24.4 | 13.6 | 15.3 | 10.1 | 10.0 |
| | PRG4 | 206007_at | 533.0 | 11.8 | 32.9 | 21.2 | 90.6 | 51.3 | 72.7 | 8.2 |
| | PTHLH | 210355_at | 797.4 | 54.8 | 162.6 | 3.1 | 35.0 | 31.9 | 11.0 | 3.9 |
| | SERPING1 | 200986_at | 138.9 | 11.8 | 17.9 | 35.2 | 34.3 | 35.5 | 18.8 | 10.9 |
| Intracellular | ANXA3 | 209369_at | 1396.8 | 21.6 | 9.5 | 170.6 | 81.3 | 102.5 | 28.3 | 25.0 |
| | C10orf10 | 209183_s_at | 340.3 | 11.0 | 7.6 | 23.7 | 25.0 | 20.0 | 5.6 | 4.1 |
| | CHMP4C | 226803_at | 191.8 | 4.1 | 5.4 | 3.9 | 4.4 | 8.8 | 3.4 | 3.2 |
| | DGKI | 206806_at | 157.4 | 20.6 | 7.6 | 10.8 | 9.0 | 10.2 | 6.5 | 3.6 |
| | DPYSL4 | 205493_s_at | 142.1 | 3.7 | 10.3 | 17.0 | 9.1 | 15.5 | 5.6 | 7.8 |
| | EFS | 204400_at | 94.3 | 14.0 | 18.7 | 7.1 | 17.0 | 9.8 | 12.2 | 16.7 |
| | GBP4 | 235574_at | 530.3 | 3.5 | 6.8 | 9.5 | 90.9 | 3.5 | 4.6 | 3.5 |
| | INSC | 237056_at | 145.5 | 24.4 | 11.3 | 44.1 | 30.0 | 57.4 | 27.0 | 17.6 |
| | MYH6, | 204737_s_at | 54.8 | 6.8 | 12.0 | 11.0 | 15.5 | 13.5 | 4.9 | 4.6 |
| | MYH7 | | | | | | | | | |
| | MYO5B | 225301_s_at | 342.7 | 25.3 | 7.7 | 8.1 | 10.3 | 23.1 | 7.2 | 4.3 |
| | NEXN | 1552309_a_at | 271.8 | 3.5 | 9.3 | 17.4 | 3.7 | 3.8 | 4.8 | 3.2 |
| | PCGF2 | 214239_x_at | 496.1 | 22.2 | 9.6 | 4.8 | 8.5 | 7.2 | 4.1 | 7.9 |
| | S100A9 | 203535_at | 262.3 | 18.5 | 4.7 | 16.3 | 34.9 | 29.3 | 48.6 | 27.9 |
| | STAG3 | 219753_at | 1497.8 | 8.7 | 16.0 | 20.2 | 13.1 | 14.8 | 4.3 | 8.0 |
| | XAF1 | 206133_at | 2600.6 | 15.0 | 19.9 | 22.5 | 55.8 | 16.9 | 6.2 | 4.2 |
| | ZNF503 | 227195_at | 141.4 | 6.0 | 9.3 | 3.7 | 9.3 | 12.4 | 8.0 | 13.8 |
| Unknown | DNAJC12 | 218976_at | 2695.2 | 6.4 | 5.8 | 3.4 | 13.7 | 8.9 | 3.5 | 3.7 |
| | | 223721_s_at | 1109.9 | 49.7 | 155.8 | 115.7 | 114.9 | 77.0 | 3.8 | 54.3 |
| | | 223722_at | 399.5 | 10.6 | 6.3 | 3.5 | 20.5 | 8.0 | 3.2 | 4.1 |
| | DOK5 | 1554863_s_at | 671.7 | 10.0 | 60.8 | 10.9 | 48.5 | 31.5 | 22.1 | 4.1 |
| | LMO4 | 209204_at | 263.3 | 5.4 | 3.1 | 5.4 | 7.3 | 3.7 | 3.4 | 5.6 |
| | LOC400043 | 226582_at | 196.4 | 9.6 | 4.2 | 16.3 | 15.5 | 7.1 | 6.6 | 10.7 |
| | MYO1B | 212364_at | 346.3 | 3.4 | 5.0 | 26.8 | 57.0 | 13.3 | 5.9 | 11.9 |
| | NAPSB | 228055_at | 1571.6 | 78.7 | 10.9 | 19.8 | 290.3 | 161.7 | 9.5 | 21.7 |
| | | 228056_s_at | 408.4 | 114.5 | 3.4 | 4.2 | 244.9 | 111.4 | 34.8 | 13.7 |
| | RASSF4 | 49306_at | 53.1 | 8.6 | 7.5 | 9.2 | 5.5 | 5.9 | 3.1 | 3.8 |
| | | 226436_at | 137.1 | 5.0 | 10.7 | 23.9 | 18.6 | 22.1 | 12.7 | 17.6 |
| | SH3TC2 | 219710_at | 110.8 | 19.0 | 5.7 | 8.1 | 9.0 | 10.4 | 29.5 | 11.3 |
| | SPEF2 | 232745_x_at | 83.5 | 17.8 | 3.8 | 3.3 | 5.7 | 7.9 | 4.9 | 15.8 |
| | YAP1 | 224894_at | 262.4 | 6.0 | 3.9 | 3.9 | 7.7 | 8.7 | 28.4 | 6.8 |
| | — | 239653_at | 70.2 | 5.3 | 5.3 | 10.1 | 11.2 | 8.8 | 6.4 | 3.9 |
| | — | 242203_at | 365.7 | 9.3 | 23.6 | 18.1 | 16.1 | 22.2 | 10.9 | 9.8 |
| | — | 244125_at | 108.6 | 5.9 | 15.9 | 29.6 | 29.2 | 24.3 | 5.6 | 31.8 |

TABLE 13

| | | | Relative | | | | | | | |
|---|---|---|---|---|---|---|---|---|---|---|
| | | | fluorescence | Expression ratio | | | | | | |
| | Gene | Probe Set | intensity | Tfh (2)/ | Tfh (2)/ | Tfh (2)/ | | | | |
| Localization | symbol | ID | Tfh (2) | Naïve | Th1 | Th17 | Tfh (2)/Th2 | Tfh (2)/Th22 | Tfh (2)/Th9 | Tfh (2)/Treg |
| Membrane | ART3 | 210147_at | 181.3 | 11.5 | 3.7 | 6.2 | 11.4 | 9.5 | 3.5 | 4.3 |
| | C3orf52 | 219474_at | 1842.7 | 3.7 | 5.5 | 3.9 | 10.6 | 5.7 | 11.4 | 7.5 |
| | ELOVL7 | 227180_at | 523.8 | 90.4 | 24.3 | 88.1 | 32.9 | 114.9 | 100.2 | 12.9 |
| | EMP2 | 225078_at | 91.1 | 7.0 | 10.6 | 9.9 | 11.4 | 16.7 | 5.7 | 14.0 |
| | FLT1 | 226498_at | 121.7 | 5.6 | 19.7 | 19.1 | 13.1 | 4.5 | 18.3 | 9.2 |
| | GPR26 | 1553864_at | 16.0 | 8.8 | 7.6 | 9.8 | 7.6 | 3.1 | 4.7 | 3.1 |
| | IL23R | 1552912_a_at | 358.9 | 45.5 | 27.8 | 3.4 | 51.5 | 33.4 | 56.9 | 13.9 |
| | | 1561853_a_at | 180.9 | 22.0 | 31.5 | 5.4 | 27.6 | 17.8 | 36.8 | 14.1 |
| | KCNS3 | 205968_at | 152.2 | 15.3 | 11.2 | 11.4 | 8.9 | 19.6 | 5.9 | 19.8 |
| | LHFP | 218656_s_at | 768.8 | 29.3 | 28.3 | 34.8 | 10.2 | 16.6 | 15.4 | 10.6 |
| | MARS | 213672_at | 354.8 | 8.7 | 8.5 | 12.4 | 7.3 | 9.2 | 7.0 | 7.7 |
| | SGMS2 | 227038_at | 551.9 | 60.0 | 18.7 | 14.5 | 116.1 | 97.3 | 41.8 | 9.5 |
| | SGPP2 | 244780_at | 498.3 | 62.0 | 7.3 | 4.7 | 30.0 | 7.2 | 55.8 | 3.6 |
| | SORBS1 | 218087_s_at | 74.6 | 7.5 | 13.2 | 3.7 | 7.3 | 10.9 | 3.4 | 3.8 |
| | THBS1 | 201110_s_at | 552.5 | 63.5 | 103.8 | 15.8 | 95.1 | 14.5 | 6.1 | 3.5 |
| | TM4SF1 | 209386_at | 720.8 | 24.8 | 19.7 | 34.7 | 20.0 | 11.5 | 11.2 | 14.0 |
| | | 209387_s_at | 433.8 | 40.5 | 37.5 | 124.2 | 80.3 | 38.0 | 16.9 | 55.7 |
| | | 215034_s_at | 307.6 | 37.0 | 52.5 | 88.0 | 18.8 | 32.0 | 6.9 | 62.3 |
| | TMTC1 | 226322_at | 123.9 | 26.1 | 10.8 | 46.2 | 8.7 | 24.5 | 38.4 | 7.6 |
| | TUSC3 | 213423_x_at | 122.9 | 8.8 | 9.9 | 10.0 | 6.2 | 29.6 | 20.5 | 5.6 |
| Extracellular/ | APOD | 201525_at | 512.2 | 13.9 | 8.6 | 11.2 | 9.5 | 16.5 | 6.1 | 5.9 |
| secretory | CCL18 | 209924_at | 125.7 | 6.7 | 8.7 | 9.7 | 13.3 | 11.2 | 9.9 | 3.8 |
| | | 32128_at | 161.4 | 8.4 | 12.9 | 20.2 | 12.9 | 3.4 | 11.8 | 5.1 |
| | CXCL10 | 204533_at | 5184.1 | 106.0 | 11.2 | 16.0 | 4.9 | 3.8 | 98.0 | 32.1 |
| | CXCL11 | 210163_at | 566.7 | 62.7 | 18.3 | 10.4 | 22.5 | 34.1 | 68.4 | 26.3 |
| | | 211122_s_at | 457.7 | 49.9 | 30.2 | 12.9 | 63.7 | 48.2 | 22.0 | 30.0 |
| | CXCL13 | 205242_at | 3567.7 | 764.1 | 1143.5 | 19.8 | 398.6 | 708.5 | 75.0 | 91.1 |
| | CXCL9 | 203915_at | 364.8 | 5.8 | 3.8 | 10.9 | 9.8 | 10.9 | 9.7 | 4.9 |
| | FGF2 | 204422_s_at | 86.2 | 6.5 | 3.6 | 12.9 | 23.2 | 7.8 | 11.6 | 11.5 |
| | IL2 | 207849_at | 121.5 | 7.9 | 51.6 | 20.1 | 37.3 | 8.2 | 4.1 | 11.1 |
| | MMP12 | 204580_at | 339.0 | 21.4 | 5.2 | 9.5 | 16.5 | 16.2 | 62.9 | 15.9 |
| | PPIA | 217602_at | 543.6 | 4.6 | 5.3 | 6.8 | 4.2 | 4.6 | 8.2 | 7.6 |
| | PTHLH | 206300_s_at | 517.7 | 36.7 | 22.6 | 10.3 | 41.0 | 24.4 | 37.1 | 6.4 |
| | | 210355_at | 464.1 | 16.9 | 18.8 | 8.2 | 59.0 | 36.0 | 11.6 | 7.9 |
| | | 211756_at | 1459.3 | 75.4 | 102.4 | 12.0 | 259.7 | 206.5 | 758.9 | 5.6 |
| | SERPING1 | 200986_at | 66.2 | 6.8 | 11.3 | 15.8 | 8.5 | 22.5 | 12.3 | 5.1 |
| | SPARC | 200665_s_at | 590.8 | 26.2 | 20.1 | 4.8 | 60.1 | 15.0 | 9.9 | 6.2 |
| | STC2 | 203438_at | 520.7 | 11.0 | 12.4 | 13.3 | 11.9 | 17.7 | 3.5 | 20.1 |
| Intracellular | ALDH1L2 | 231202_at | 157.8 | 40.4 | 6.2 | 7.0 | 21.3 | 15.0 | 35.2 | 13.8 |
| | ANXA3 | 209369_at | 760.0 | 15.2 | 4.9 | 65.6 | 74.4 | 41.6 | 33.1 | 9.8 |
| | DGKI | 206806_at | 97.4 | 6.7 | 9.9 | 5.0 | 7.7 | 4.8 | 4.6 | 5.9 |
| | EFS | 204400_at | 53.9 | 5.4 | 6.9 | 9.5 | 12.4 | 7.5 | 10.2 | 13.3 |
| | FERMT2 | 209209_s_at | 159.5 | 30.6 | 35.2 | 10.8 | 68.3 | 15.0 | 12.9 | 7.8 |
| | | 209210_s_at | 3068.1 | 213.4 | 33.2 | 10.8 | 102.5 | 37.1 | 44.9 | 62.1 |
| | HIST1H1A | 208484_at | 101.0 | 10.0 | 10.7 | 12.0 | 7.4 | 6.0 | 3.1 | 13.5 |
| | IRF4 | 216987_at | 95.8 | 13.9 | 16.7 | 23.2 | 50.4 | 63.0 | 6.2 | 38.3 |
| | KLF4 | 221841_s_at | 207.7 | 10.6 | 5.0 | 5.2 | 4.2 | 8.1 | 8.5 | 5.0 |
| | METTL3 | 213653_at | 995.8 | 5.3 | 4.2 | 5.2 | 3.8 | 5.5 | 6.6 | 7.3 |
| | MYH6 | 214468_at | 463.2 | 19.9 | 12.8 | 19.4 | 18.4 | 14.9 | 70.2 | 9.6 |
| | MYH6, | 204737_s_at | 247.6 | 68.0 | 50.1 | 65.9 | 103.0 | 156.9 | 75.3 | 60.8 |
| | MYH7 | | | | | | | | | |
| | PCGF2 | 214239_x_at | 392.5 | 10.8 | 11.1 | 3.3 | 13.4 | 12.5 | 5.0 | 8.3 |
| | PHLDA2 | 209803_s_at | 2496.5 | 16.7 | 3.8 | 7.2 | 5.3 | 8.7 | 4.8 | 8.5 |
| | PNMA2 | 209598_at | 278.1 | 4.8 | 6.4 | 7.7 | 12.7 | 6.5 | 5.5 | 9.8 |
| | POLR1C | 209317_at | 3959.1 | 3.2 | 3.5 | 5.2 | 4.6 | 3.8 | 5.0 | 3.8 |
| | RAI14 | 202052_s_at | 146.4 | 12.3 | 24.9 | 28.1 | 34.3 | 25.7 | 10.6 | 12.2 |
| | RGS20 | 210138_at | 168.7 | 32.2 | 53.4 | 37.5 | 14.5 | 32.4 | 17.0 | 19.3 |
| | RPL10 | 221989_at | 1958.4 | 5.1 | 3.4 | 3.6 | 3.1 | 4.5 | 3.6 | 5.3 |
| | RTKN | 225150_s_at | 96.9 | 10.1 | 3.2 | 4.4 | 9.2 | 18.5 | 5.1 | 3.3 |
| | SPHK1 | 219257_s_at | 360.0 | 4.2 | 4.0 | 5.1 | 10.2 | 9.7 | 36.0 | 3.7 |
| | TEAD4 | 41037_at | 186.9 | 26.5 | 29.5 | 7.7 | 18.4 | 16.9 | 6.8 | 4.4 |
| | UCHL1 | 201387_s_at | 2711.2 | 38.0 | 41.7 | 14.5 | 18.6 | 27.6 | 3.4 | 12.4 |
| | WDR74 | 218278_at | 2164.7 | 3.8 | 9.0 | 9.3 | 8.5 | 7.5 | 3.4 | 4.5 |
| Unknown | CA13 | 231270_at | 448.9 | 5.1 | 5.0 | 3.6 | 3.9 | 6.7 | 4.8 | 3.4 |
| | CT45A5 | 235700_at | 390.6 | 36.1 | 24.2 | 18.2 | 10.9 | 14.2 | 9.9 | 25.2 |
| | DNAJC12 | 218976_at | 10104.8 | 35.6 | 18.9 | 8.1 | 38.7 | 37.8 | 15.5 | 11.2 |
| | | 223721_s_at | 1292.3 | 52.0 | 110.8 | 73.1 | 224.2 | 214.7 | 5.5 | 27.0 |
| | | 223722_at | 1546.2 | 54.5 | 29.6 | 12.8 | 55.9 | 89.9 | 20.6 | 16.5 |
| | DOK5 | 1554863_s_at | 390.0 | 36.7 | 17.1 | 14.1 | 22.5 | 20.2 | 33.2 | 3.6 |
| | EFR3B | 227283_at | 330.0 | 6.6 | 7.4 | 4.6 | 12.0 | 9.2 | 11.9 | 7.4 |
| | GAS5 | 227517_s_at | 7076.1 | 7.6 | 3.5 | 5.5 | 4.4 | 6.3 | 4.2 | 6.7 |
| | LOC339751 | 1558653_at | 133.8 | 20.8 | 9.2 | 6.8 | 10.5 | 40.0 | 26.9 | 38.9 |
| | MAK | 220302_at | 474.5 | 17.3 | 10.6 | 34.3 | 5.8 | 23.2 | 5.2 | 3.5 |
| | MIR155HG | 229437_at | 6622.5 | 10.4 | 12.7 | 65.0 | 11.4 | 19.2 | 4.8 | 8.0 |
| | MYO1B | 212364_at | 400.8 | 4.3 | 6.5 | 42.9 | 16.3 | 22.5 | 6.3 | 16.5 |
| | | 212365_at | 282.4 | 3.9 | 3.3 | 4.2 | 5.1 | 10.0 | 3.6 | 5.3 |
| | RIBC2 | 206526_at | 286.2 | 5.1 | 28.3 | 20.3 | 15.4 | 21.9 | 38.4 | 8.8 |
| | RIMKLA | 241075_at | 411.1 | 28.9 | 7.1 | 4.2 | 4.2 | 13.7 | 20.2 | 17.6 |
| | USP12 | 215886_x_at | 349.0 | 6.7 | 4.5 | 5.0 | 6.8 | 5.3 | 3.3 | 4.2 |
| | YAP1 | 213342_at | 120.3 | 8.2 | 6.3 | 7.6 | 14.9 | 6.2 | 7.9 | 7.7 |
| | | 224894_at | 392.1 | 13.1 | 6.1 | 5.7 | 17.6 | 10.1 | 44.0 | 15.4 |
| | | 224895_at | 135.4 | 3.1 | 3.0 | 4.0 | 8.4 | 5.6 | 4.9 | 5.6 |
| | ZBED2 | 219836_at | 19643.9 | 31.2 | 3.9 | 4.3 | 40.8 | 23.5 | 5.1 | 3.2 |
| | — | 230180_at | 2427.6 | 3.9 | 5.2 | 5.8 | 4.7 | 3.6 | 3.5 | 4.4 |
| | — | 232277_at | 391.7 | 25.8 | 8.2 | 11.9 | 10.2 | 26.2 | 46.6 | 17.5 |
| | — | 232667_at | 103.6 | 3.1 | 10.9 | 4.9 | 12.8 | 10.6 | 6.8 | 4.0 |
| | — | 241726_at | 716.2 | 25.6 | 24.4 | 4.5 | 25.6 | 32.7 | 44.6 | 42.6 |
| | — | 244434_at | 19.0 | 15.0 | 7.9 | 17.4 | 15.3 | 17.2 | 6.0 | 8.4 |
| | — | 1556773_at | 336.0 | 36.5 | 50.9 | 14.4 | 50.5 | 39.3 | 29.4 | 5.1 |

TABLE 14

| | | | Relative | | | | | | | |
|---|---|---|---|---|---|---|---|---|---|---|
| | | | fluorescence | Expression ratio | | | | | | |
| | Gene | Probe Set | intensity | Tfh (2)/ | Tfh (2)/ | Tfh (2)/ | Tfh (2)/ | | | |
| Localization | symbol | ID | Tfh (2) | Naïve | Th1 | Th17 | Th2 | Tfh (2)/Th22 | Tfh (2)/Th9 | Tfh (2)/Treg |
| Membrane | B4GALT6 | 206233_at | 145.1 | 10.6 | 36.8 | 15.2 | 26.6 | 14.2 | 3.0 | 7.5 |
| | CLIC2 | 213415_at | 231.7 | 12.4 | 3.1 | 8.9 | 9.2 | 8.7 | 4.1 | 7.6 |
| | EMP2 | 225078_at | 91.6 | 10.9 | 9.2 | 8.8 | 8.1 | 13.1 | 15.1 | 20.6 |
| | EPHA4 | 206114_at | 293.6 | 7.2 | 8.1 | 3.9 | 24.4 | 46.0 | 90.8 | 11.7 |
| | GAP43 | 204471_at | 386.8 | 54.8 | 65.9 | 42.3 | 102.5 | 46.6 | 3.1 | 40.5 |
| | IL23R | 1552912_a_at | 498.2 | 71.0 | 34.8 | 4.1 | 112.7 | 14.0 | 17.8 | 10.9 |
| | | 1561853_a_at | 233.2 | 56.9 | 54.4 | 3.6 | 28.1 | 38.6 | 9.9 | 22.3 |
| | KCNS3 | 205968_at | 99.9 | 14.5 | 5.6 | 10.0 | 7.3 | 22.7 | 13.3 | 8.3 |
| | KITLG | 226534_at | 488.1 | 25.2 | 5.2 | 4.0 | 5.8 | 7.3 | 23.8 | 16.3 |
| | LHFP | 218656_s_at | 1726.4 | 65.1 | 59.7 | 46.4 | 16.9 | 23.8 | 19.5 | 20.7 |
| | MAP1B | 212233_at | 176.8 | 4.1 | 7.1 | 4.5 | 3.8 | 8.9 | 3.4 | 6.5 |
| | | 226084_at | 135.9 | 16.0 | 11.4 | 7.5 | 6.3 | 5.6 | 4.1 | 4.1 |
| | PAWR | 204005_s_at | 136.9 | 3.2 | 13.2 | 7.8 | 21.5 | 5.2 | 3.7 | 4.5 |
| | TM4SF1 | 209386_at | 1218.9 | 31.2 | 42.2 | 43.3 | 76.8 | 19.5 | 15.0 | 25.2 |
| | | 209387_s_at | 830.9 | 82.1 | 38.5 | 235.0 | 80.4 | 42.3 | 19.3 | 64.0 |
| | | 215034_s_at | 672.5 | 94.6 | 48.0 | 149.4 | 33.1 | 88.9 | 24.3 | 106.9 |
| | TMTC1 | 224397_s_at | 44.4 | 3.6 | 3.4 | 8.5 | 4.9 | 5.8 | 17.1 | 11.0 |
| | | 226322_at | 138.1 | 43.7 | 15.3 | 29.6 | 14.5 | 42.7 | 15.6 | 7.9 |
| | TUSC3 | 213423_x_at | 129.5 | 3.4 | 13.5 | 10.3 | 12.0 | 14.1 | 8.6 | 9.0 |
| Extracellular/ | APOD | 201525_at | 414.3 | 9.5 | 6.1 | 7.3 | 6.7 | 7.1 | 4.9 | 3.9 |
| secretory | CXCL11 | 210163_at | 593.9 | 19.5 | 11.7 | 7.2 | 20.9 | 11.3 | 34.9 | 18.3 |
| | | 211122_s_at | 476.4 | 23.2 | 23.9 | 8.7 | 42.0 | 11.2 | 38.0 | 36.0 |
| | CXCL13 | 205242_at | 3809.9 | 840.7 | 1039.1 | 21.9 | 883.7 | 848.0 | 26.7 | 104.6 |
| | FGF1 | 205117_at | 187.7 | 4.7 | 5.3 | 3.8 | 4.9 | 3.2 | 6.3 | 6.1 |
| | INHBA | 210511_s_at | 487.7 | 21.3 | 17.7 | 29.0 | 43.5 | 17.8 | 56.2 | 53.8 |
| | MMP12 | 204580_at | 365.6 | 54.4 | 4.9 | 9.0 | 31.2 | 8.8 | 11.2 | 24.4 |
| | PTHLH | 210355_at | 1543.8 | 123.0 | 364.8 | 7.1 | 78.6 | 71.5 | 24.7 | 8.6 |
| | SPARC | 200665_s_at | 382.1 | 10.4 | 17.9 | 6.3 | 32.2 | 12.0 | 6.7 | 5.6 |
| | TNFRSF11B | 204932_at | 604.0 | 25.1 | 39.8 | 9.7 | 25.0 | 31.8 | 16.7 | 20.2 |
| | | 204933_s_at | 155.6 | 8.0 | 12.2 | 20.2 | 17.1 | 13.2 | 12.2 | 11.2 |
| Intracellular | ALDH1L2 | 231202_at | 95.6 | 37.4 | 5.8 | 3.7 | 17.9 | 20.4 | 9.1 | 8.7 |
| | ANXA3 | 209369_at | 773.4 | 12.1 | 5.3 | 95.4 | 45.5 | 57.4 | 15.8 | 14.0 |
| | DGKI | 206806_at | 263.9 | 38.5 | 14.1 | 20.3 | 16.9 | 19.1 | 12.2 | 6.7 |
| | EFS | 204400_at | 93.3 | 15.3 | 20.6 | 7.8 | 18.7 | 10.7 | 13.4 | 18.3 |
| | FERMT2 | 209209_s_at | 249.8 | 12.1 | 7.3 | 8.3 | 11.4 | 6.3 | 5.5 | 13.9 |
| | | 209210_s_at | 4842.7 | 31.3 | 13.3 | 9.0 | 32.1 | 19.2 | 25.7 | 46.4 |
| | KLF4 | 221841_s_at | 422.0 | 6.9 | 4.7 | 9.8 | 4.0 | 5.6 | 10.7 | 8.1 |
| | MYH6 | 214468_at | 328.4 | 11.7 | 11.0 | 17.2 | 30.2 | 9.3 | 49.6 | 4.5 |
| | MYH6, | 204737_s_at | 161.9 | 38.7 | 68.1 | 62.2 | 87.4 | 76.3 | 27.5 | 26.1 |
| | MYH7 | | | | | | | | | |
| | MYO5B | 225301_s_at | 353.8 | 26.2 | 8.0 | 8.4 | 10.7 | 23.9 | 7.5 | 4.4 |
| | PLOD2 | 202620_s_at | 417.0 | 22.8 | 13.2 | 4.8 | 15.0 | 24.4 | 10.4 | 14.8 |
| | PNMA2 | 209598_at | 249.6 | 5.6 | 18.7 | 5.0 | 7.8 | 9.9 | 4.4 | 8.0 |
| | RAI14 | 202052_s_at | 223.9 | 38.0 | 62.6 | 57.4 | 47.3 | 37.3 | 18.7 | 55.9 |
| | RGS20 | 210138_at | 173.8 | 41.9 | 22.0 | 3.4 | 17.0 | 20.2 | 12.0 | 33.2 |
| | TEAD4 | 41037_at | 181.6 | 13.4 | 13.7 | 4.7 | 19.6 | 3.4 | 13.8 | 3.6 |
| | TOM1L1 | 204485_s_at | 421.0 | 27.1 | 9.1 | 3.5 | 8.6 | 3.9 | 3.8 | 9.6 |
| | UCHL1 | 201387_s_at | 2288.2 | 52.4 | 37.3 | 12.9 | 18.4 | 31.6 | 3.4 | 13.1 |
| Unknown | C8orf47 | 1552390_a_at | 72.7 | 6.9 | 16.6 | 15.1 | 12.0 | 9.6 | 3.0 | 4.9 |
| | CA13 | 231270_at | 712.2 | 5.9 | 7.9 | 7.0 | 10.3 | 12.0 | 5.8 | 4.0 |
| | CCDC77 | 224521_s_at | 813.2 | 4.0 | 3.5 | 3.6 | 3.4 | 4.6 | 4.4 | 3.0 |
| | CT45A1, | 1567912_s_at | 63.0 | 19.9 | 7.4 | 5.9 | 32.3 | 6.2 | 11.9 | 3.2 |
| | CT45A2, | | | | | | | | | |
| | CT45A3, | | | | | | | | | |
| | CT45A4, | | | | | | | | | |
| | CT45A5, | | | | | | | | | |
| | CT45A6, | | | | | | | | | |
| | LOC100133581 | | | | | | | | | |
| | CT45A5 | 235700_at | 346.5 | 9.0 | 7.5 | 20.8 | 7.3 | 25.5 | 10.3 | 18.4 |
| | DNAJC12 | 218976_at | 9918.0 | 23.7 | 21.6 | 12.6 | 51.0 | 33.3 | 13.0 | 13.9 |
| | | 223721_s_at | 1195.1 | 53.2 | 166.9 | 123.9 | 123.1 | 82.4 | 4.1 | 58.2 |
| | | 223722_at | 1282.6 | 33.4 | 19.6 | 10.9 | 64.3 | 25.2 | 10.2 | 12.9 |
| | DOK5 | 214844_s_at | 8541.1 | 14.1 | 55.1 | 7.3 | 136.8 | 123.0 | 61.5 | 4.8 |
| | | 1554863_s_at | 967.5 | 14.4 | 87.8 | 15.8 | 70.0 | 45.5 | 32.0 | 5.9 |
| | EFR3B | 227283_at | 315.6 | 8.3 | 5.0 | 4.3 | 13.0 | 11.6 | 8.8 | 4.3 |
| | FLJ31958 | 232803_at | 226.3 | 3.3 | 4.7 | 4.5 | 4.7 | 4.8 | 3.5 | 3.3 |
| | KIAA0895 | 213424_at | 136.3 | 5.2 | 13.6 | 3.5 | 4.2 | 6.1 | 7.0 | 5.0 |
| | LOC340184 | 1563589_at | 164.1 | 8.3 | 7.3 | 4.6 | 12.8 | 22.6 | 6.8 | 8.6 |
| | LOC400043 | 226582_at | 102.0 | 9.8 | 4.3 | 16.6 | 15.8 | 7.2 | 6.7 | 10.9 |
| | MYO1B | 212364_at | 374.5 | 4.2 | 6.2 | 33.1 | 70.4 | 16.5 | 7.3 | 14.7 |
| | OSBPL6 | 223805_at | 198.2 | 17.9 | 3.7 | 6.4 | 6.3 | 6.6 | 8.4 | 6.2 |
| | RIBC2 | 206526_at | 188.7 | 5.0 | 28.7 | 27.1 | 23.6 | 31.0 | 48.8 | 13.3 |
| | RIMKLA | 236884_at | 174.0 | 5.3 | 5.8 | 3.4 | 4.7 | 3.7 | 4.4 | 4.1 |
| | | 241075_at | 386.4 | 19.8 | 16.0 | 7.2 | 10.0 | 29.3 | 13.6 | 12.5 |
| | SPEF2 | 232745_x_at | 146.8 | 31.3 | 6.7 | 5.7 | 10.0 | 13.8 | 8.5 | 27.8 |
| | YAP1 | 213342_at | 117.4 | 4.7 | 8.8 | 12.7 | 14.8 | 19.4 | 9.1 | 3.9 |
| | | 224894_at | 380.6 | 9.8 | 6.3 | 6.3 | 12.4 | 14.1 | 46.1 | 11.1 |
| | — | 227140_at | 893.6 | 9.8 | 7.9 | 9.9 | 21.2 | 25.6 | 46.9 | 26.7 |
| | — | 232277_at | 688.7 | 19.1 | 14.1 | 23.6 | 13.9 | 12.9 | 238.8 | 55.6 |
| | — | 241726_at | 472.6 | 31.0 | 19.4 | 3.9 | 29.7 | 15.0 | 14.6 | 10.8 |

In the present Example, the genes which were found to be specifically expressed in Tfh cells by Chtanova T. et al. (see Chtanova T. et al., supra), i.e. SGPP2 (sphingosine-1-phosphate phosphatase 2), CXCL13 (chemokine (C-X-C motif) ligand 13), IL-21 (interleukin 21) and MAF (v-maf musculoaponeurotic fibrosarcoma oncogene homolog) were also identified. These known genes are shown in Table 15.

TABLE 15

| | | | | | | Tfh (1) | | Tfh (2) | |
|---|---|---|---|---|---|---|---|---|---|
| Gene | Entrez | Protein | Transcript | UniGene | Probe Set | w/o | w/ CD2/3/28 | w/o | w/ CD2/3/28 |
| symbol | Gene ID | ID | ID | ID | ID | stimulation | stimulation | stimulation | stimulation |
| SGPP2 | 130367 | NP_689599 | NM_152386 | Hs.591604 | 244780_at | | | | |
| CXCL13 | 10563 | NP_006410 | NM_006419 | Hs.100431 | 205242_at | | | | |
| IL21 | 59067 | NP_068575 | NM_021803 | Hs.567559 | 221271_at | | | | |
| MAF | 4094 | NP_001026974, | NM_001031804, | Hs.134859 | 209347_s_at | | | | |
| | | NP_005351 | NM_005360 | | | | | | |

As described above, the genes which have been known to be specifically expressed in Tfh cells could also be detected in the present Example. Therefore, it is apparent that the genes specifically expressed in Tfh cells can be identified according to the present approach for gene search using the Tfh cells and other subsets of Th cells as described above.

The present inventors have identified 161 genes which are specifically expressed in Tfh cells corresponding to the genes identified as above excluding 4 genes shown in Table 15 as the polynucleotide markers for detecting Tfh cells according to the present invention.

The list of the genes as the present polynucleotide marker is shown in Table 16. In Table 16, the genes marked with “.” are specifically expressed in Tfh (1) and Tfh (2) cells with or without activation stimulation with anti-CD2/3/28 antibody.

TABLE 16-1

| | | | | | | | | SEQ | | | | |
|---|---|---|---|---|---|---|---|---|---|---|---|---|
| | | Gene | Entrez | Protein | Transcript | UniGene | Probe Set | ID | Tfh (1) | | Tfh (2) | |
| Localization | No. | symbol | Gene ID | ID | ID | ID | ID | NO: | w/o stimulation | w/ CD2/3/28 stimulation | w/o stimulation | w/ CD2/3/28 stimulation |
| Membrane | 1 | ART3 | 419 | NP_001123488, | NM_001130016, | Hs.24976 | 210147_at | 1 | | | | |
| | | | | NP_001123489, | NM_001130017, | | | | | | | |
| | | | | NP_001170 | NM_001179 | | | | | | | |
| | 2 | B4GALT6 | 9331 | NP_004766 | NM_004775 | Hs.591063 | 206233_at | 2 | | | | |
| | 3 | C3orf52 | 79669 | NP_078892 | NM_024616 | Hs.434247 | 219474_at | 3 | | | | |
| | 4 | CD28 | 940 | NP_006130 | NM_006139 | Hs.591629 | 211861_x_at | 4 | | | | |
| | 5 | CD9 | 928 | NP_001760 | NM_001769 | Hs.114286 | 201005_at | 5 | | | | |
| | 6 | CDH3 | 1001 | NP_001784 | NM_001793 | Hs.191842 | 203256_at | 6 | | | | |
| | 7 | CLIC2 | 1193 | NP_001280 | NM_001289 | Hs.655445 | 213415_at | 7 | | | | |
| | 8 | ELOVL7 | 79993 | NP_001098028, | NM_001104558, | Hs.274256 | 227180_at | 8 | | | | |
| | | | | NP_079206 | NM_024930 | | | | | | | |
| | 9 | EMP2 | 2013 | NP_001415 | NM_001424 | Hs.531561 | 225078_at | 9 | | | | |
| | | | | | | | 225079_at | 10 | | | | |
| | 10 | EPHA4 | 2043 | NP_004429 | NM_004438 | Hs.371218 | 206114_at | 11 | | | | |
| | 11 | FAM26F | 441168 | NP_001010919 | NM_001010919 | Hs.381220 | 229543_at | 12 | | | | |
| | 12 | FCGR1B | 2210 | NP_001004340, | NM_001004340, | Hs.534956 | 214511_x_at | 13 | | | | |
| | | | | NP_001017986 | NM_001017986 | | | | | | | |
| | 13 | FLT1 | 2321 | NP_001153392, | NM_001159920, | Hs.654360 | 226498_at | 14 | | | | |
| | | | | NP_001153502, | NM_001160030, | | | | | | | |
| | | | | NP_001153503, | NM_001160031, | | | | | | | |
| | | | | NP_002010 | NM_002019 | | | | | | | |
| | 14 | FUT7 | 2529 | NP_004470 | NM_004479 | — | 210506_at | 15 | | | | |
| | 15 | GABBR1, | 10537, | NP_001461, | NM_001470, | Hs.167017 | 205890_s_at | 16 | | | | |
| | | UBD | 2550 | NP_006389, | NM_006398, | | | | | | | |
| | | | | NP_068703, | NM_021903, | | | | | | | |
| | | | | NP_068704, | NM_021904, | | | | | | | |
| | | | | NP_068705 | NM_021905 | | | | | | | |
| | 16 | GAP43 | 2596 | NP_001123536, | NM_001130064, | Hs.134974 | 204471_at | 17 | | | | |
| | | | | NP_002036 | NM_002045 | | | | | | | |
| | 17 | GPC4 | 2239 | NP_001439 | NM_001448 | Hs.58367 | 204984_at | 18 | | | | |
| | 18 | GPR26 | 2849 | NP_703143 | NM_153442 | Hs.12751 | 1553864_at | 19 | | | | |
| | 19 | GPR84 | 53831 | NP_065103 | NM_020370 | Hs.306199 | 223767_at | 20 | | | | |
| | 20 | IL12RB2 | 3595 | NP_001550 | NM_001559 | Hs.479347 | 206999_at | 21 | | | | |
| | 21 | IL23R | 149233 | NP_653302 | NM_144701 | Hs.677426 | 1552912_a_at | 22 | | | | |
| | | | | | | | 1561853_a_at | 23 | | | | |
| | 22 | KCNS3 | 3790 | NP_002243 | NM_002252 | Hs.414489 | 205968_at | 24 | | | | |
| | 23 | KIAA1244 | 57221 | NP_065073 | NM_020340 | Hs.656215 | 231856_at | 25 | | | | |
| | 24 | KISS1R | 84634 | NP_115940 | NM_032551 | Hs.208229 | 242517_at | 26 | | | | |
| | 25 | KITLG | 4254 | NP_000890, | NM_000899, | Hs.1048 | 226534_at | 27 | | | | |
| | | | | NP_003985 | NM_003994 | | | | | | | |
| | 26 | LHFP | 10186 | NP_005771 | NM_005780 | Hs.507798 | 218656_s_at | 28 | | | | |
| | 27 | LST1 | 7940 | NP_009092, | NM_007161, | Hs.436066 | 211582_x_at | 29 | | | | |
| | | | | NP_995309, | NM_205837, | | 214181_x_at | 30 | | | | |
| | | | | NP_995310, | NM_205838, | | | | | | | |
| | | | | NP_995311, | NM_205839, | | | | | | | |
| | | | | NP_995312 | NM_205840 | | | | | | | |
| | 28 | MAP1B | 4131 | NP_005900 | NM_005909 | Hs.335079 | 212233_at | 31 | | | | |
| | | | | | | | 226084_at | 32 | | | | |
| | 29 | MARS | 4141 | NP_004981 | NM_004990 | Hs.632707 | 213672_at | 33 | | | | |
| | 30 | NTRK3 | 4916 | NP_001007157, | NM_001007156, | Hs.410969 | 215025_at | 34 | | | | |
| | | | | NP_001012338, | NM_001012338, | | | | | | | |
| | | | | NP_002521 | NM_002530 | | | | | | | |
| | 31 | PAWR | 5074 | NP_002574 | NM_002583 | Hs.643130 | 204005_s_at | 35 | | | | |
| | 32 | PLSCR1 | 5359 | NP_066928 | NM_021105 | Hs.130759 | 202430_s_at | 36 | | | | |
| | | | | | | | 202446_s_at | 37 | | | | |
| | 33 | PPP2CB | 5516 | NP_001009552, | NM_001009552, | Hs.491440 | 201374_x_at | 38 | | | | |
| | | | | NP_004147 | NM_004156 | | | | | | | |
| | 34 | RHOU | 58480 | NP_067028 | NM_021205 | Hs.647774 | 223169_s_at | 39 | | | | |
| | 35 | SGMS2 | 166929 | NP_001129729, | NM_001136257, | Hs.595423 | 227038_at | 40 | | | | |
| | | | | NP_001129730, | NM_001136258, | | | | | | | |
| | | | | NP_689834 | NM_152621 | | | | | | | |
| | 36 | SLC35E4 | 339665 | NP_001001479 | NM_001001479 | Hs.715651 | 1568623_a_at | 41 | | | | |
| Membrane | 37 | SORBS1 | 10580 | NP_001030126, | NM_001034954, | Hs.719081 | 218087_s_at | 42 | | | | |
| | | | | NP_001030127, | NM_001034955, | | | | | | | |
| | | | | NP_001030128, | NM_001034956, | | | | | | | |
| | | | | NP_001030129, | NM_001034957, | | | | | | | |
| | | | | NP_006425, | NM_006434, | | | | | | | |
| | | | | NP_056200, | NM_015385, | | | | | | | |
| | | | | NP_079267 | NM_024991 | | | | | | | |
| | 38 | TAC1 | 6863 | NP_003173, | NM_003182, | Hs.2563 | 206552_s_at | 43 | | | | |
| | | | | NP_054702, | NM_013996, | | | | | | | |
| | | | | NP_054703, | NM_013997, | | | | | | | |
| | | | | NP_054704 | NM_013998 | | | | | | | |
| | 39 | THBS1 | 7057 | NP_003237 | NM_003246 | Hs.164226 | 201109_s_at | 44 | | | | |
| | | | | | | | 201110_s_at | 45 | | | | |
| | 40 | TM4SF1 | 4071 | NP_055035 | NM_014220 | Hs.715499 | 209386_at | 46 | | | | |
| | | | | | | | 209387_s_at | 47 | | | | |
| | | | | | | | 215034_s_at | 48 | | | | |
| | 41 | TMCC2 | 9911 | NP_055673 | NM_014858 | Hs.6360 | 213096_at | 49 | | | | |
| | 42 | TMEM213 | 155006 | NP_001078898 | NM_001085429 | Hs.567729 | 239593_at | 50 | | | | |
| | | | | | | | 240183_at | 51 | | | | |
| | 43 | TMTC1 | 83857 | NP_787057 | NM_175861 | Hs.401954 | 224397_s_at | 52 | | | | |
| | | | | | | | 226322_at | 53 | | | | |
| | 44 | TUSC3 | 7991 | NP_006756, | NM_006765, | Hs.591845 | 213423_x_at | 54 | | | | |
| | | | | NP_839952 | NM_178234 | | | | | | | |
| Extracellular/ | 45 | APOD | 347 | NP_001638 | NM_001647 | Hs.522555 | 201525_at | 55 | | | | |
| secretory | 46 | CCL18 | 6362 | NP_002979 | NM_002988 | Hs.143961 | 209924_at | 56 | | | | |
| | | | | | | | 32128_at | 57 | | | | |
| | 47 | CCL2 | 6347 | NP_002973 | NM_002982 | Hs.303649 | 216598_s_at | 58 | | | | |
| | 48 | COL6A2 | 1292 | NP_001840, | NM_001849, | Hs.420269 | 209156_s_at | 59 | | | | |
| | | | | NP_478054, | NM_058174, | | | | | | | |
| | | | | NP_478055 | NM_058175 | | | | | | | |
| | 49 | CSF2 | 1437 | NP_000749 | NM_000758 | Hs.1349 | 210229_s_at | 60 | | | | |
| | 50 | CXCL10 | 3627 | NP_001556 | NM_001565 | Hs.632586 | 204533_at | 61 | | | | |
| | 51 | CXCL11 | 6373 | NP_005400 | NM_005409 | Hs.632592 | 210163_at | 62 | | | | |
| | | | | | | | 211122_s_at | 63 | | | | |
| | 52 | CXCL9 | 4283 | NP_002407 | NM_002416 | Hs.77367 | 203915_at | 64 | | | | |
| | 53 | FGF1 | 2246 | NP_000791, | NM_000800, | Hs.483635 | 205117_at | 65 | | | | |
| | | | | NP_001138364, | NM_001144892, | | | | | | | |
| | | | | NP_001138406, | NM_001144934, | | | | | | | |
| | | | | NP_001138407, | NM_001144935, | | | | | | | |
| | | | | NP_149127, | NM_033136, | | | | | | | |
| | | | | NP_149128 | NM_033137, | | | | | | | |
| | | | | | NR_026695, | | | | | | | |
| | | | | | NR_026696 | | | | | | | |
| | 54 | FGF18 | 8817 | NP_003853 | NM_003862 | Hs.87191 | 206987_x_at | 66 | | | | |
| | | | | | | | 211029_x_at | 67 | | | | |
| | 55 | FGF2 | 2247 | NP_001997 | NM_002006 | Hs.284244 | 204422_s_at | 68 | | | | |
| | 56 | IFNB1 | 3456 | NP_002167 | NM_002176 | Hs.93177 | 208173_at | 69 | | | | |
| | 57 | IL2 | 3558 | NP_000577 | NM_000586 | Hs.89679 | 207849_at | 70 | | | | |
| | 58 | IL8 | 3576 | NP_000575 | NM_000584 | Hs.624 | 211506_s_at | 71 | | | | |
| | 59 | INHBA | 3624 | NP_002183 | NM_002192 | Hs.583348 | 210511_s_at | 72 | | | | |
| | 60 | MDK | 4192 | NP_001012333, | NM_001012333, | Hs.82045 | 209035_at | 73 | | | | |
| | | | | NP_001012334, | NM_001012334, | | | | | | | |
| | | | | NP_002382 | NM_002391 | | | | | | | |
| Extracellular/ | 61 | MMP12 | 4321 | NP_002417 | NM_002426 | Hs.1695 | 204580_at | 74 | | | | |
| secretory | 62 | PPIA | 5478 | NP_066953 | NM_021130 | — | 217602_at | 75 | | | | |
| | 63 | PRG4 | 10216 | NP_001121180, | NM_001127708, | Hs.647723 | 206007_at | 76 | | | | |
| | | | | NP_001121181, | NM_001127709, | | | | | | | |
| | | | | NP_001121182, | NM_001127710, | | | | | | | |
| | | | | NP_005798 | NM_005807 | | | | | | | |
| | 64 | PTHLH | 5744 | NP_002811, | NM_002820, | Hs.591159 | 206300_s_at | 77 | | | | |
| | | | | NP_945315, | NM_198964, | | 210355_at | 78 | | | | |
| | | | | NP_945316, | NM_198965, | | 211756_at | 79 | | | | |
| | | | | NP_945317 | NM_198966 | | | | | | | |
| | 65 | SERPING1 | 710 | NP_000053, | NM_000062, | Hs.384598 | 200986_at | 80 | | | | |
| | | | | NP_001027466 | NM_001032295 | | | | | | | |
| | 66 | SPARC | 6678 | NP_003109 | NM_003118 | Hs.111779 | 200665_s_at | 81 | | | | |
| | 67 | STC2 | 8614 | NP_003705 | NM_003714 | Hs.233160 | 203438_at | 82 | | | | |
| | 68 | TNFRSF11B | 4982 | NP_002537 | NM_002546 | Hs.81791 | 204932_at | 83 | | | | |
| | | | | | | | 204933_s_at | 84 | | | | |
| Intracellular | 69 | ALDH1L2 | 160428 | NP_001029345 | NM_001034173, | Hs.42572 | 231202_at | 85 | | | | |
| | | | | | NR_027752 | | | | | | | |
| | 70 | ANXA3 | 306 | NP_005130 | NM_005139 | Hs.480042 | 209369_at | 86 | | | | |
| | 71 | BSPRY | 54836 | NP_060158 | NM_017688 | Hs.614517 | 218792_s_at | 87 | | | | |
| | | | | | | | 222746_s_at | 88 | | | | |
| | 72 | C10orf10 | 11067 | NP_008952 | NM_007021 | Hs.93675 | 209183_s_at | 89 | | | | |
| | 73 | CHMP4C | 92421 | NP_689497 | NM_152284 | Hs.183861 | 226803_at | 90 | | | | |
| | 74 | DGKI | 9162 | NP_004708 | NM_004717 | Hs.242947 | 206806_at | 91 | | | | |
| | 75 | DPYSL4 | 10570 | NP_006417 | NM_006426 | Hs.100058 | 205493_s_at | 92 | | | | |
| | 76 | EFS | 10278 | NP_005855, | NM_005864, | Hs.24587 | 204400_at | 93 | | | | |
| | | | | NP_115835 | NM_032459 | | | | | | | |
| | 77 | ELL | 8178 | NP_006523 | NM_006532 | Hs.515260 | 1565254_s_at | 94 | | | | |
| | 78 | FBXO17, | 115290, | NP_001139373, | NM_001145901, | Hs.709416 | 220233_at | 95 | | | | |
| | | SARS2 | 54938 | NP_060297, | NM_017827, | | | | | | | |
| | | | | NP_079183, | NM_024907, | | | | | | | |
| | | | | NP_680474 | NM_148169 | | | | | | | |
| | 79 | FERMT2 | 10979 | NP_001128471, | NM_001134999, | Hs.509343 | 209209_s_at | 96 | | | | |
| | | | | NP_001128472, | NM_001135000, | | 209210_s_at | 97 | | | | |
| | | | | NP_006823 | NM_006832 | | | | | | | |
| | 80 | GBP4 | 115361 | NP_443173 | NM_052941 | Hs.409925 | 235574_at | 98 | | | | |
| | 81 | HIST1H1A | 3024 | NP_005316 | NM_005325 | Hs.150206 | 208484_at | 99 | | | | |
| | 82 | INSC | 387755 | NP_001027024, | NM_001031853, | Hs.591997 | 237056_at | 100 | | | | |
| | | | | NP_001036001 | NM_001042536 | | | | | | | |
| | 83 | IRF4 | 3662 | NP_002451 | NM_002460 | Hs.401013 | 216987_at | 101 | | | | |
| | 84 | KLF4 | 9314 | NP_004226 | NM_004235 | Hs.376206 | 221841_s_at | 102 | | | | |
| | 85 | MAST4 | 375449 | NP_055998, | NM_015183, | Hs.595458 | 1554652_s_at | 103 | | | | |
| | | | | NP_942123 | NM_198828 | | | | | | | |
| | 86 | METTL3 | 56339 | NP_062826 | NM_019852 | Hs.168799 | 213653_at | 104 | | | | |
| Intracellular | 87 | MGST1 | 4257 | NP_064696, | NM_020300, | Hs.389700 | 1565162_s_at | 105 | | | | |
| | | | | NP_665707, | NM_145764, | | | | | | | |
| | | | | NP_665734, | NM_145791, | | | | | | | |
| | | | | NP_665735 | NM_145792 | | | | | | | |
| | 88 | MYH6 | 4624 | NP_002462 | NM_002471 | Hs.278432 | 214468_at | 106 | | | | |
| | 89 | MYH6, | 4624, | NP_002462, | NM_002471, | Hs.278432 | 204737_s_at | 107 | | | | |
| | | MYH7 | 4625 | NP_000248 | NM_000257 | | | | | | | |
| | 90 | MYO5B | 4645 | NP_001073936 | NM_001080467 | Hs.200136 | 225301_s_at | 108 | | | | |
| | 91 | NEXN | 91624 | NP_653174 | NM_144573 | Hs.612385 | 1552309_a_at | 109 | | | | |
| | 92 | PCGF2 | 7703 | NP_009075 | NM_007144 | Hs.371617 | 214239_x_at | 110 | | | | |
| | 93 | PHLDA2 | 7262 | NP_003302 | NM_003311 | Hs.154036 | 209803_s_at | 111 | | | | |
| | 94 | PLOD2 | 5352 | NP_000926, | NM_000935, | Hs.477866 | 202619_s_at | 112 | | | | |
| | | | | NP_891988 | NM_182943 | | 202620_s_at | 113 | | | | |
| | 95 | PNMA2 | 10687 | NP_009188 | NM_007257 | Hs.591838 | 209598_at | 114 | | | | |
| | 96 | POLR1C | 9533 | NP_004866, | NM_004875, | Hs.719353 | 209317_at | 115 | | | | |
| | | | | NP_976035 | NM_203290 | | | | | | | |
| | 97 | RAI14 | 26064 | NP_001138992, | NM_001145520, | Hs.431400 | 202052_s_at | 116 | | | | |
| | | | | NP_001138993, | NM_001145521, | | | | | | | |
| | | | | NP_001138994, | NM_001145522, | | | | | | | |
| | | | | NP_001138995, | NM_001145523, | | | | | | | |
| | | | | NP_001138997, | NM_001145525, | | | | | | | |
| | | | | NP_056392 | NM_015577 | | | | | | | |
| | 98 | RGS20 | 8601 | NP_003693, | NM_003702, | Hs.368733 | 210138_at | 117 | | | | |
| | | | | NP_733466 | NM_170587 | | | | | | | |
| | 99 | RPL10 | 6134 | NP_006004 | NM_006013, | Hs.534404 | 221989_at | 118 | | | | |
| | | | | | NR_026898 | | | | | | | |
| | 100 | RTKN | 6242 | NP_001015055, | NM_001015055, | Hs.192854 | 225150_s_at | 119 | | | | |
| | | | | NP_001015056, | NM_001015056, | | | | | | | |
| | | | | NP_149035 | NM_033046 | | | | | | | |
| | 101 | S100A9 | 6280 | NP_002956 | NM_002965 | Hs.112405 | 203535_at | 120 | | | | |
| | 102 | SOCS3 | 9021 | NP_003946 | NM_003955 | Hs.527973 | 206359_at | 121 | | | | |
| | 103 | SPHK1 | 8877 | NP_001136073, | NM_001142601, | Hs.68061 | 219257_s_at | 122 | | | | |
| | | | | NP_001136074, | NM_001142602, | | | | | | | |
| | | | | NP_068807, | NM_021972, | | | | | | | |
| | | | | NP_892010 | NM_182965 | | | | | | | |
| | 104 | STAG3 | 10734 | NP_036579 | NM_012447 | Hs.592283 | 219753_at | 123 | | | | |
| | 105 | TEAD4 | 7004 | NP_003204, | NM_003213, | Hs.94865 | 41037_at | 124 | | | | |
| | | | | NP_958849, | NM_201441, | | | | | | | |
| | | | | NP_958851 | NM_201443 | | | | | | | |
| | 106 | TOM1L1 | 10040 | NP_005477 | NM_005486 | Hs.153504 | 204485_s_at | 125 | | | | |
| | 107 | UCHL1 | 7345 | NP_004172 | NM_004181 | Hs.518731 | 201387_s_at | 126 | | | | |
| | 108 | VPS53 | 55275 | NP_001121631, | NM_001128159, | Hs.461819 | 219794_at | 127 | | | | |
| | | | | NP_060759 | NM_018289 | | | | | | | |
| | 109 | WDR74 | 54663 | NP_060563 | NM_018093 | Hs.655372 | 218278_at | 128 | | | | |
| | 110 | XAF1 | 54739 | NP_059993, | NM_017523, | Hs.441975 | 206133_at | 129 | | | | |
| | | | | NP_954590 | NM_199139 | | | | | | | |
| | 111 | ZNF334 | 55713 | NP_060572, | NM_018102, | Hs.584933 | 220022_at | 130 | | | | |
| | | | | NP_955473 | NM_199441 | | | | | | | |
| | 112 | ZNF503 | 84858 | NP_116161 | NM_032772 | — | 227195_at | 131 | | | | |
| Unknown | 113 | C8orf47 | 203111 | NP_775820 | NM_173549 | Hs.171455 | 1552390_a_at | 132 | | | | |
| | 114 | CA13 | 377677 | NP_940986 | NM_198584 | Hs.127189 | 231270_at | 133 | | | | |
| | 115 | CCDC77 | 84318 | NP_001123618, | NM_001130146, | Hs.631656 | 224521_s_at | 134 | | | | |
| | | | | NP_001123619, | NM_001130147, | | | | | | | |
| | | | | NP_001123620, | NM_001130148, | | | | | | | |
| | | | | NP_115734 | NM_032358 | | | | | | | |
| | 116 | CT45A1, | 100133581, | NP_001007552, | NM_001007551, | Hs.460937 | 1567912_s_at | 135 | | | | |
| | | CT45A2, | 441519, | NP_001017417, | NM_001017417, | | | | | | | |
| | | CT45A3, | 441520, | NP_001017435, | NM_001017435, | | | | | | | |
| | | CT45A4, | 441521, | NP_001017436, | NM_001017436, | | | | | | | |
| | | CT45A5, | 541465, | NP_001017438, | NM_001017438, | | | | | | | |
| | | CT45A6, | 541466, | NP_689795, | NM_152582, | | | | | | | |
| | | LOC100133581 | 728911 | XP_001716054, | XM_001716002, | | | | | | | |
| | | | | XP_002346378 | XM_002346337 | | | | | | | |
| | 117 | CT45A5 | 441521 | NP_001007552 | NM_001007551 | Hs.535081 | 235700_at | 136 | | | | |
| | 118 | DNAJC12 | 56521 | NP_068572, | NM_021800, | Hs.260720 | 218976_at | 137 | | | | |
| | | | | NP_957714 | NM_201262 | | 223721_s_at | 138 | | | | |
| | | | | | | | 223722_at | 139 | | | | |
| | 119 | DOK5 | 55816 | NP_060901 | NM_018431 | Hs.656582 | 1554863_s_at | 140 | | | | |
| | | | | | | | 214844_s_at | 141 | | | | |
| | 120 | EFR3B | 22979 | NP_055786 | NM_014971 | Hs.4892 | 227283_at | 142 | | | | |
| | 121 | FLJ31958 | 143153 | — | — | Hs.706372 | 232803_at | 143 | | | | |
| | 122 | GADD45B | 4616 | NP_056490 | NM_015675 | Hs.110571 | 209305_s_at | 144 | | | | |
| | 123 | GAS5 | 60674 | — | NR_002578 | Hs.656411 | 227517_s_at | 145 | | | | |
| | 124 | KIAA0895 | 23366 | NP_001093895, | NM_001100425, | Hs.6224 | 213424_at | 146 | | | | |
| | | | | NP_056129 | NM_015314 | | | | | | | |
| | 125 | LMO4 | 8543 | NP_006760 | NM_006769 | Hs.436792 | 209204_at | 147 | | | | |
| | 126 | LOC339751 | 339751 | — | — | Hs.623925 | 1558653_at | 148 | | | | |
| | 127 | LOC340184 | 340184 | — | — | — | 1563589_at | 149 | | | | |
| | 128 | LOC400043 | 400043 | — | NR_026656 | Hs.19193 | 226582_at | 150 | | | | |
| | 129 | MAK | 4117 | NP_005897 | NM_005906 | Hs.446125 | 220302_at | 151 | | | | |
| | 130 | MIR155HG | 114614 | — | NR_001458 | Hs.697120 | 229437_at | 152 | | | | |
| | 131 | MYO1B | 4430 | NP_001123630, | NM_001130158, | Hs.439620 | 212364_at | 153 | | | | |
| | | | | NP_001155291, | NM_001161819, | | 212365_at | 154 | | | | |
| | | | | NP_036355 | NM_012223 | | | | | | | |
| | 132 | NAPSB | 256236 | — | NR_002798 | Hs.636624 | 228055_at | 155 | | | | |
| | | | | | | | 228056_s_at | 156 | | | | |
| | 133 | NPHP1 | 4867 | NP_000263, | NM_000272, | Hs.280388 | 238844_s_at | 157 | | | | |
| | | | | NP_001121650, | NM_001128178, | | | | | | | |
| | | | | NP_001121651, | NM_001128179, | | | | | | | |
| | | | | NP_997064 | NM_207181 | | | | | | | |
| | 134 | OSBPL6 | 114880 | NP_115912, | NM_032523, | Hs.318775 | 223805_at | 158 | | | | |
| | | | | NP_665682 | NM_145739 | | | | | | | |
| | 135 | RASSF4 | 83937 | NP_114412 | NM_032023 | Hs.522895 | 226436_at | 159 | | | | |
| | | | | | | | 49306_at | 160 | | | | |
| | 136 | RIBC2 | 26150 | NP_056468 | NM_015653 | Hs.475110 | 206526_at | 161 | | | | |
| | 137 | RIMKLA | 284716 | NP_775913 | NM_173642 | Hs.420244 | 236884_at | 162 | | | | |
| | | | | | | | 241075_at | 163 | | | | |
| Unknown | 138 | SH3TC2 | 79628 | NP_078853 | NM_024577 | Hs.483784 | 219710_at | 164 | | | | |
| | 139 | SPEF2 | 79925 | NP_079143, | NM_024867, | Hs.298863 | 232745_x_at | 165 | | | | |
| | | | | NP_653323 | NM_144722 | | | | | | | |
| | 140 | USP12 | 219333 | NP_872294 | NM_182488 | Hs.42400 | 215886_x_at | 166 | | | | |
| | 141 | YAP1 | 10413 | NP_001123617, | NM_001130145, | Hs.503692 | 213342_at | 167 | | | | |
| | | | | NP_006097 | NM_006106 | | 224894_at | 168 | | | | |
| | | | | | | | 224895_at | 169 | | | | |
| | 142 | ZBED2 | 79413 | NP_078784 | NM_024508 | Hs.136912 | 219836_at | 170 | | | | |
| | 143 | — | — | — | — | Hs.670716 | 1556773_at | 171 | | | | |
| | 144 | — | — | — | — | Hs.684722 | 1561654_at | 172 | | | | |
| | 145 | — | — | — | — | Hs.675457 | 215314_at | 173 | | | | |
| | 146 | — | — | — | — | Hs.28792 | 227140_at | 174 | | | | |
| | 147 | — | — | — | — | Hs.705517 | 230180_at | 175 | | | | |
| | 148 | — | — | — | — | Hs.535966 | 232277_at | 176 | | | | |
| | 149 | — | — | — | — | Hs.618995 | 232667_at | 177 | | | | |
| | 150 | — | — | — | — | Hs.656964 | 239653_at | 178 | | | | |
| | 151 | — | — | — | — | Hs.606172 | 241726_at | 179 | | | | |
| | 152 | — | — | — | — | Hs.593315 | 242203_at | 180 | | | | |
| | 153 | — | — | — | — | Hs.601938 | 244125_at | 181 | | | | |
| | 154 | — | — | — | — | Hs.567457 | 244434_at | 182 | | | | |

As apparent from Table 16, 161 genes of the present invention are useful as the polynucleotide markers for detecting Tfh cells.

### Example 2

### Expression Assay of Protein Markers for Detecting

Tfh cells in human peripheral blood-derived cultured Th1, Th2 and Tfh cells

### 1. Preparation of Th Cells

Tfh cells, Th1 cells and Th2 cells in human peripheral blood were separated with a cell sorter (FACS Aria: Becton Dickinson). The separated respective Th cells were seeded in a 96-well plate at a density of 1.0×10⁵ cells/0.3 ml/well. The medium use was the Yssel medium. In order to allow activation and proliferation of Th cells, magnetic beads coated with an anti-CD2/3/28 antibody (Miltenyi Biotec) were added to each well at 0.5×10⁵. The cells were added with cytokines and neutralizing antibodies suitable for differentiation culture of respective Th cells and cultured in an incubator at 37° C. and 5% CO₂. The antibodies and settings for gating used for separation and culture conditions for the Th cells are the same as Example 1.

### 2. Preparation of Sample for CD9 Measurement

The prepared respective Th cells (5×10⁶ cells/ml) were added with a PE-labeled anti-human CD9 antibody (BioLegend) at a final concentration of 0.2 μg/ml and incubated at 4° C. for 20 minutes. After the reaction, the Th cells were added with PBS containing 0.5% BSA and recovered by centrifugation. The washed Th cells were suspended in PBS containing 0.5 μg/ml 7-aminoactinomycin D (7-AAD) and 0.5% BSA to prepare a sample for CD9 measurement (5×10⁶ cells/ml). A negative control sample (5×10⁶ cells/ml) was prepared by adding a PE-labeled murine IgG 1 isotype control (Biolegend) at a final concentration of 1.0 μg/ml instead of the anti-CD9 antibody and incubating at 4° C. for 20 minutes.

### 3. Preparation of Sample for CXCR5 Measurement

Samples for CXCR5 measurement for the respective Th cells (5×10⁶ cells/ml) were prepared in the same manner as the preparation of sample for CD9 measurement as above 2. except that a PerCP/Cy5.5-labeled anti-CXCR5 antibody (BD Biosciences) at a final concentration of 1.0 μg/ml was used instead of the PE-labeled anti-CD9 antibody.

A negative control sample (5×10⁶ cells/ml) was prepared by adding a PerCP/Cy5.5-labeled anti-mouse IgG2b antibody (BioLegend) at a final concentration of 1.0 μg/ml instead of the PerCP/Cy5.5-labeled anti-CXCR5 antibody and incubating at 4° C. for 20 minutes.

### 4. Preparation of Sample for IL-21 Measurement

In order to measure IL-21 positive cells in the prepared respective Th cell samples, samples for measurement of IL-21 were prepared from the respective samples. To the samples which were adjusted to 2.5×10⁵ cells/ml were added phorbol myristate acetate (PMA: 50 ng/ml) and ionomycin (1 μM) to stimulate the cells. After 4 hours, the cells were added with brefeldin A (10 μg/ml) and cultured for additional 2 hours. The cells were washed with phosphate buffered saline (PBS) and fixed with 4% paraformaldehyde. After fixation, the cells were treated with a saponin buffer (0.5% saponin, 0.5% bovine serum albumin (BSA), 1 mM sodium azide (in PBS)) in order to increase permeability of the cell membrane. The cells were allowed to react with a fluorescence (PE)-labeled anti-IL-21 antibody. After the reaction, the cells were washed with the saponin buffer and then with the 0.5% BSA-containing PBS and then suspended in the 0.5% BSA-containing PBS to prepare the samples for IL-21 measurement.

A negative control sample was prepared by adding the PE-labeled anti-mouse IgG 1 antibody (BioLegend) at a final concentration of 1.0 μg/ml instead of the PE-labeled anti-IL-21 antibody and incubating at 4° C. for 20 minutes.

### 5. Expression Assay of CD9 Protein Using Flow Cytometer

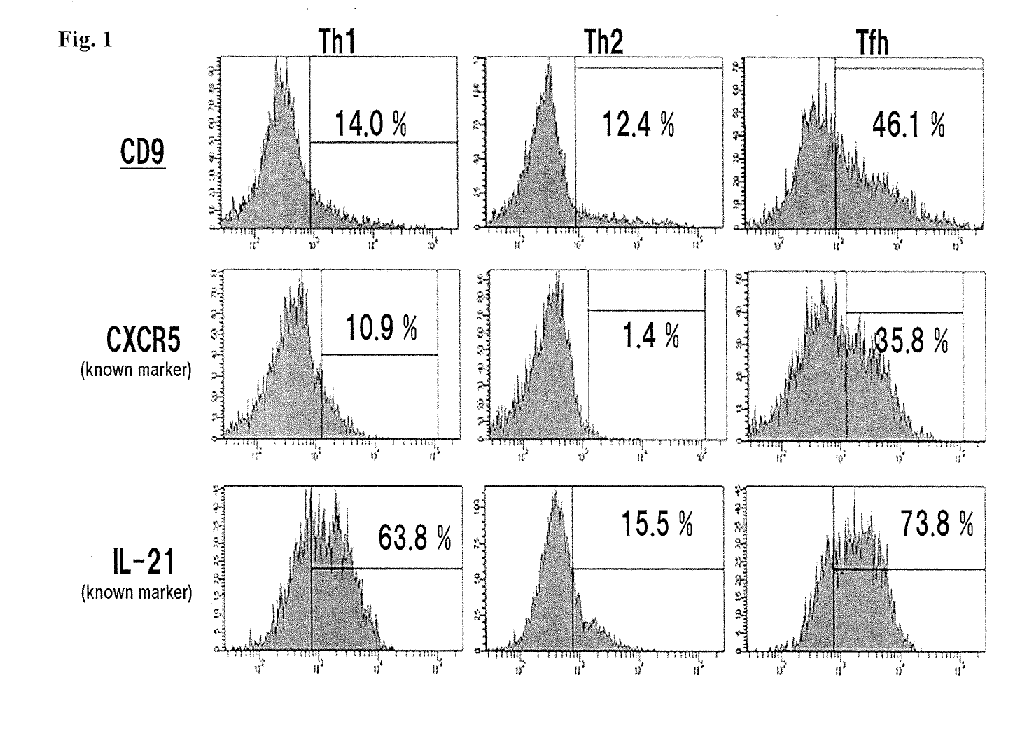

The samples for CD9 measurement, CXCR5 measurement and IL-21 measurement prepared as above were analyzed with the FACSCanto II (BD Biosciences) and FACS DIVA software (BD Biosciences). The histograms (particle distribution) of fluorescence intensity obtained from the analyses are shown in FIG. 1. In FIG. 1, the vertical axis of histograms represents the number of cells and the horizontal axis represents fluorescence intensity. The numbers shown in upper right of the histograms represent the ratio (%) of marker positive cells relative to total cells in the respective samples. The positive cells and negative cells were determined based on the maximum fluorescence intensity in negative controls. Namely, the cells having the fluorescence intensity higher than the maximum fluorescence intensity in a negative control were determined as positive cells. On the other hand, the cells having the fluorescence intensity at or lower than the maximum fluorescence intensity in the negative control were determined as negative cells. The positive cell ratio was calculated as the ratio of the positive cells relative to total number of cells.

According to FIG. 1, it is found that CD9 is specifically and highly expressed in Tfh cells than in Th1 cells and Th2 cells. It is also found that the positive cell ratio of Tfh cells in the sample for CD9 measurement is higher than the sample for CXCR5 measurement, which is a known marker for Tfh cells. Moreover, the positive cell ratio of the sample for IL-21 measurement, which is a known marker for Tfh cells, is high for not only Tfh cells but also for Th1 cells, while the positive cell ratio of the sample for CD9 measurement is low for Th1 cells and Th2 cells and high only for Tfh cells. In summary, it is found that CD9 protein can be suitably used as a protein marker for detecting Tfh cells.

### Example 3

### Analysis of Tfh Cell Ratio in Tfh Cell Detection Marker Fraction

### 1. Preparation of Fractionated Sample

The cultured Tfh cells prepared in Example 2 were stained with the PerCP/Cy5.5-labeled anti-CXCR5 antibody to prepare a sample for CXCR5 measurement. The cultured Tfh cells were stained with the PerCP/Cy5.5-labeled anti-CXCR5 antibody and the PE-labeled anti-human CD9 antibody to prepare a sample for CXCR5/CD9 measurement. CXCR5 positive cells were separated from the sample for CXCR5 measurement with a cell sorter (FACS Aria: Becton Dickinson). CXCR5 positive and CD9 positive cells were separated from the sample for CXCR5/CD9 measurement with the cell sorter.

The separated cells were respectively seeded in a 96-well plate at a density of 1.5×10⁵ cells/0.3 ml/well or less. The medium used was the Yssel medium containing IL-2 (R&D systems) at a final concentration of 10 ng/ml and the cells were cultured in an incubator at 37° C. and 5% CO₂ for 3 days to prepare a CXCR5 fractionated sample and a CXCR5/CD9 fractionated sample.

### 2. Sample Processing for Measurement of IL-21 Positive Cells

In order to measure IL-21 positive cells in the cultured Tfh cell sample before fractionation as described in 1., and the CXCR5 fractionated sample and the CXCR5/CD9 fractionated sample after fractionation, samples for IL-21 measurement were prepared therefrom. To the samples adjusted to 2.5×10⁵ cells/ml were added phorbol myristate acetate (PMA: 50 ng/ml) and ionomycin (1 μM) to stimulate the cells. After 4 hours, the cells were added with brefeldin A (10 μg/ml) and cultured for additional 2 hours. The cells were then washed with phosphate buffered saline (PBS) and fixed with 4% paraformaldehyde. After fixation, the cells were treated with a saponin buffer (0.5% saponin, 0.5% bovine serum albumin (BSA), 1 mM sodium azide (in PBS)) in order to increase permeability of the cell membrane. The cells were allowed to react with a fluorescence (PE)-labeled anti-IL-21 antibody. After the reaction, the cells were washed with the saponin buffer and with the 0.5% BSA-containing PBS and then suspended in the 0.5% BSA-containing PBS to prepare the samples for IL-21 measurement.

A negative control sample was prepared by adding the PE-labeled anti-mouse IgG 1 antibody (BioLegend) at a final concentration of 1.0 μg/ml instead of the PE-labeled anti-IL-21 antibody and incubating at 4° C. for 20 minutes.

### 3. Measurement of IL-21 Positive Cells

The samples for IL-21 measurement prepared as in the above 2. were analyzed with the FACSCanto II (BD Biosciences) and FACS DIVA software (BD Biosciences). The ratio of IL-21 positive cells relative to total number of cells in the samples for IL-21 measurement was calculated to obtain the Tfh cell ratio (%).

The positive cells and negative cells were determined based on the maximum fluorescence intensity in the negative control. Namely, the cells having the fluorescence intensity higher than the maximum fluorescence intensity in the negative control were determined as positive cells.

On the other hand, the cells having the fluorescence intensity at or lower than the maximum fluorescence intensity in the negative control were determined as negative cells. The positive cell ratio was calculated as the ratio of the positive cells relative to total number of cells.

### 4. Calculation of Enrichment Ratio of Tfh Cells

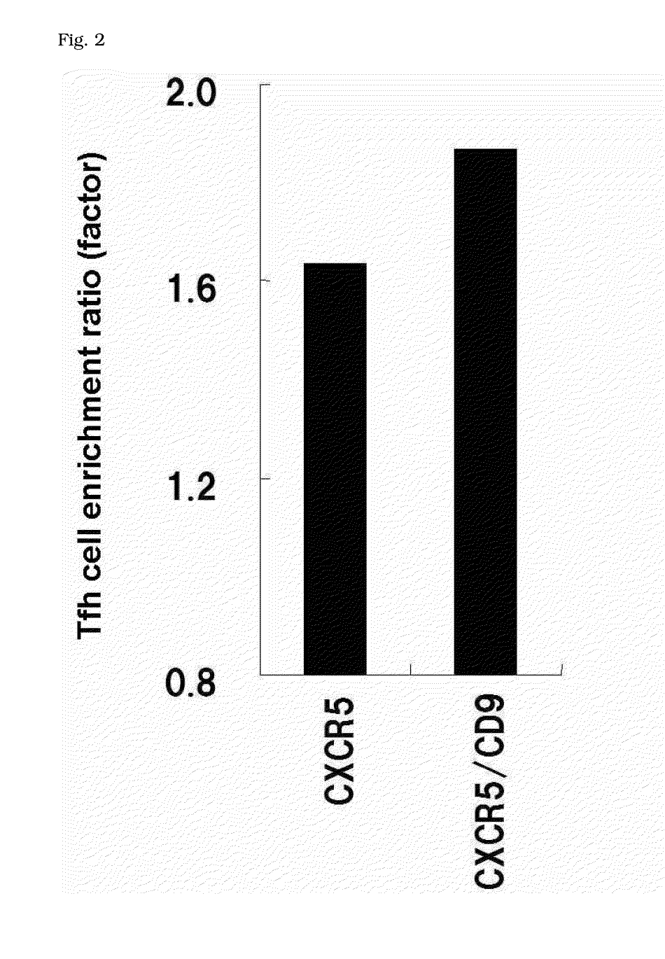

Based on the Tfh cell ratio (%) of the samples obtained in the above 3., enrichment ratio of Tfh cells in the Tfh cell detection marker fractions was calculated. Specifically, the ratio of the Tfh cell ratio (%) of the respective fractions relative to the Tfh cell ratio (%) before fractionation was calculated. The obtained ratios are shown in FIG. 2 as the enrichment ratios of Tfh cells in the CXCR5 fractionated sample and CXCR5/CD9 fractionated sample. According to FIG. 2, Tfh cells were enriched to a higher concentration in the CXCR5/CD9 fraction than CXCR5 which is a known marker for Tfh cells. Therefore, it is found that the combination of CD9 protein and CXCR5 protein is useful as a protein marker for detecting Tfh cells compared to the CXCR5 protein alone.

1. A polynucleotide marker for detecting human follicular helper T cells, which is a polynucleotide having a base sequence of at least one gene selected from:
a gene encoding a membrane protein represented by CD9, TM4SF1, IL23R, ART3, ELOVL7, KCNS3, LHFP, PAWR, THBS1, B4GALT6, C3orf52, CD28, CDH3, CLIC2, EMP2, EPHA4, FAM26F, FCGR1B, FLT1, FUT7, GABBR1, UBD, GAP43, GPC4, GPR26, GPR84, IL12RB2, KIAA1244, KISS1R, KITLG, LST1, MAP1B, MARS, NTRK3, PLSCR1, PPP2CB, RHOU, SGMS2, SLC35E4, SORBS1, TAC1, TMCC2, TMEM213, TMTC1 or TUSC3;
a gene encoding an extracellular/secretory protein represented by APOD, CXCL9, CXCL10, CXCL11, FGF2, IL2, PTHLH, SERPING1, CCL18, CCL2, COL6A2, CSF2, FGF1, FGF18, IFNB1, IL8, INHBA, MDK, MMP12, PPIA, PRG4, SPARC, STC2 or TNFRSF11B;
a gene encoding an intracellular protein represented by ANXA3, DGKI, EFS, MYH6, MYH7, MYO5B, PCGF2, PNMA2, RGS20, ALDH1L2, BSPRY, C10orf10, CHMP4C, DPYSL4, ELL, FBXO17, SARS2, FERMT2, GBP4, HIST1H1A, INSC, IRF4, KLF4, MAST4, METTL3, MGST1, NEXN, PHLDA2, PLOD2, POLR1C, RAI14, RPL10, RTKN, S100A9, SOCS3, SPHK1, STAG3, TEAD4, TOM1L1, UCHL1, VPS53, WDR74, XAF1, ZNF334 or ZNF503;
a gene represented by DNAJC12, DOK5, LOC400043, MYO1B, SPEF2, YAP1, C8orf47, CA13, CCDC77, CT45A1, CT45A2, CT45A3, CT45A4, CT45A5, CT45A6, LOC100133581, EFR3B, FLJ31958, GADD45B, GAS5, KIAA0895, LMO4, LOC339751, LOC340184, MAK, MIR155HG, NAPSB, NPHP1, OSBPL6, RASSF4, RIBC2, RIMKLA, SH3TC2, USP12 or ZBED2; and
a gene consisting of a base sequence represented by any of SEQ ID NOs: 171, 182 and 172 to 181;
or a variant or fragment thereof.

2. The polynucleotide marker according to claim 1, which is a polynucleotide having the base sequence of the gene encoding CD9 or a variant or fragment thereof.

3. A polynucleotide marker for detecting human follicular helper T cells, which is a combination of the polynucleotide marker according to claim 2 and a polynucleotide having a base sequence of a gene encoding CXCR5 or a variant or fragment thereof.

4. A protein marker for detecting human follicular helper T cells, which is a protein encoded by at least one gene selected from the genes defined in claim 1, or a functionally equivalent variant or fragment thereof.

5. A protein marker for detecting human follicular helper T cells, which is a combination of the protein marker according to claim 4 and a protein having an amino acid sequence of CXCR5 or a functionally equivalent variant or fragment thereof.

6. A method for detecting human follicular helper T cells, comprising detecting at least one polynucleotide marker for detecting human follicular helper T cells according to claim 1.

7. A protein marker for detecting human follicular helper T cells, which is a protein encoded by at least one gene selected by the gene defined in claim 2, or a functionally equivalent variant or fragment thereof.

8. A protein marker for detecting human follicular helper T cells, which is a combination of the protein marker according to claim 7 and a protein having an amino acid sequence of CXCR5 or a functionally equivalent variant or fragment thereof.

9. A method for detecting human follicular helper T cells, comprising detecting at least one protein marker for detecting human follicular helper T cells according to claim 4 in a sample containing cells.


SEQUENCE LISTING (182 sequences)

SEQ ID NO: 1 - DNA, 534 bp, Homo sapiens
gaatattttcaacccatctatgtctacaaccctggtgagaaaaaccagaagcttgaagaccatagtgagaaaaactggaagcttgaagaccatggtgagaaaaaccagaagcttgaagaccatggtgtgaaaatccttgaacccacccaaatacctgctccaggtccagttcctgttccaggtcccaaaagccatccttctgcatcctcgggcaaactgctgcttccacagtttgggatggtcatcattttaatcagtgtttctgctataaatctctttgttgctctgtagttttgatgcattgttttatctctcttattcctttacttgaaataacttatagggatccacaggagatcaaaaggaatgatgtattttttacgtgttggccaaagtcactggataaaatgagaattgtatatttgttattcattttgcaaattcagaaagttggtccagatatatgtcacagaacttttcacttgtatactactcttacaatggaaaaaaatcccgaaaactgtatacttctga

SEQ ID NO: 2 - DNA, 438 bp, Homo sapiens
gtactcgaaaaatcaaaccttctgaaaattatgatatttgtacttcatttgtgtattagttcattttctgtttctattacatagtttttaaagtcaatgaaagagattgagaatgttaacatttgttgatcaatgtggagctgtccacattcggtatcttttcatttttatatatattttacagtttcaagaaaacttagtactactagtatctgccttattttcttaagtctgatgttaatttttgtttcaggagtgagatataaacattttgctttttttcagttttgaaatatagtttgtcatcctgtgaaaaccacctcgtttgctttttgtttgtttgttttgcttttttagtgtagacctaaaattatttttgcctataaacagtgaacttaatatgtttctgtgagagtgacagagctgtgcccgtttatc

SEQ ID NO: 3 - DNA, 444 bp, Homo sapiens
cagtgtcttcaggcaatgattgtgtatcctgttacagatggttgtgatacaagaagaaccactcttctttgaaaataaactcttggaagcttttgccagctatctggggggtaggaggaatattagcaactgtattggttgtctacagatacagaattgcctgttgtgagggaactgattgttttgttgggaaaagaaatttaccaggagaaagagttttgtgctgtattgtgagagatctcgcctctcagttaaatgagccctgggttaaagtcagtgtgaagggcagctgtgtgcgggcacgagccagagtgtctgcctcagactagatttgacttgagttctttatgacccaggactctggataatgtgaatttgctttcctatttaactagaagatacatgtactatagatcattgtctcattttagtgattgttcctta

SEQ ID NO: 4 - DNA, 156 bp, Homo sapiens
tctcaacttattcccttcaattcaagtaacaggaaacaagattttggtgaagcagtcgcccatgcttgtagcgtacgacaatgcggtcaaccttagctacaatgagaagagcaatggaaccattatccatgtgaaagggaaacacctttgtccaag

SEQ ID NO: 5 - DNA, 185 bp, Homo sapiens
ctgatgccatcaaagaggtcttcgacaataaattccacatcatcggcgcagtgggcatcggcattgccgtggtcatgatatttggcatgatcttcagtatgatcttgtgctgtgctatccgcaggaaccgcgagatggtctagagtcagcttacatccctgagcaggaaagtttacccatgaaga

SEQ ID NO: 6 - DNA, 477 bp, Homo sapiens
gtggccgtagcaacttggcggagacaggctatgagtctgacgttagagtggttgcttccttagcctttcaggatggaggaatgtgggcagtttgacttcagcactgaaaacctctccacctgggccagggttgcctcagaggccaagtttccagaagcctcttacctgccgtaaaatgctcaaccctgtgtcctgggcctgggcctgctgtgactgacctacagtggactttctctctggaatggaaccttcttaggcctcctggtgcaacttaatttttttttttaatgctatcttcaaaacgttagagaaagttcttcaaaagtgcagcccagagctgctgggcccactggccgtcctgcatttctggtttccagaccccaatgcctcccattcggatggatctctgcgtttttatactgagtgtgcctaggttgccccttattttttattttccctgttgcgttgctatagatg

SEQ ID NO: 7 - DNA, 518 bp, Homo sapiens
  misc_feature (159)..(166): n is a, c, g, or t
ttcttctatccaactctcttatgaaaaggaactctgtattttctattagccataaataatctgtccactgtattttacaggtcttcatacttttacttaattttctttatctgtatggcaaaccactgcaatcctgaatgacatggaaagcatcacaannnnnnnncctttgcttgaattcctggaatgcatacatataagctaaacagatgtctgcagttataaatgtcataagtagaggtacaatctcaccctgctccttagaaacatttccatataaatcgctaaaataatttcacatttttgttagtttaatatatacatgagtttatttctgatataaataataaatacagagagtgagcatatcagagaggcaaattcttaaagaatgatttttaaaatcagctctaggaagagctcaagatcaattggtcatagaacagcatttgacgcctagaactatgaccacctcatggtcagagatgagaatgtagcctttgtgaccagattatatt

SEQ ID NO: 8 - DNA, 493 bp, Homo sapiens
  misc_feature (35)..(35): n is a, c, g, or t
taacctccctgtagtctttaaaatgtttaacttgnctataagctaacatgtaataaaacacatactcaattatatgaatagaagtggagagccagaatgctacaaaagaaatcacagctgctagaagtatcctccatagaaacatcaacatgattgtggatcaaaatgattttcactggatatggaatttgtatgggccatatttattaaaagagttctgtgtggtcacatagagttcctttgggatttcatcccacttttcaggacttaatttgtttgggtttgcttacctaacaagcaacaacaacaacaaaataaatagttccaaaatctagtttattacataaatctctatgaatccttgagggcctatatctatattataaataaatacaaatatagatttttaaatatctatgggactttgccatttacagccttaagtataaaattacgagattatattctttccattaccctttatttctgctaa

SEQ ID NO: 9 - DNA, 395 bp, Homo sapiens
  misc_feature (28)..(28): n is a, c, g, or t
gagaatgcttgacttgttggtctgtgtnaatgtgtatatatatatanncctgaatacaggaacatcggagacctattcactcccacacactctgctatagtttgcgtgcttttgtggacacccctcatgaacaggctggcgctctaggacgctctgtgttcactgatgatgaagaaacctagaactccaagcctgtttgtaaacacactaaacacagtggcctagatagaaactgtatcgtagtttaaaatctgcctcgcgggatgttactaaactcgctaatagtttaaaggttacttacaatagagcaagttggacaattttgtggtgttggggaaatgttagggcaaggcctagaggttcattttgaatcttggtttgtgactttagggtag

SEQ ID NO: 10 - DNA, 476 bp, Homo sapiens
  misc_feature (181)..(181): n is a, c, g, or t
tctggccttctataattttctggtagtcacgatggaaacaaacaaaacaccttagaaccagagatcgaccccctcaagcaatacatcaattcccttcacaagaaacgtcggggctacatgagtatctgtgttgaatgcggtctgaaatgatcctatggattttcccggctggttgccactnctgtacaacattcagtgcccacatccanctgtgccattaagcttttttgagacatgagagatgcctcttccctgctgtatgacatgcatttgggaagttggaaagaaatgacaaaatcagggagaaaacatccaagcttcttacctgtagatagaatcagccctcacttggtgcttattaccagttattcaagaacaataacaacaacaaaattagtagacatccaagaagcacatattaggaccaaagatagcatcaactgtatttgaaggaactgtagtttgcgcattttatg

SEQ ID NO: 11 - DNA, 375 bp, Homo sapiens
gcaaccccaacagcttgaagaggacagggacggagagctccagacctaacactgccttgttggatccaagctcccctgaattctctgctgtggtatcagtgggcgattggctccaggccattaaaatggaccggtataaggataacttcacagctgctggttataccacactagaggctgtggtgcacgtgaaccaggaggacctggcaagaattggtatcacagccatcacgcaccagaataagattttgagcagtgtccaggcaatgcgaacccaaatgcagcagatgcacggcagaatggttcccgtctgagccagtactgaataaactcaaaactcttgaaattagtttacctcatccatgcactttaatt

SEQ ID NO: 12 - DNA, 136 bp, Homo sapiens
tctactcattcaaaaggtcataactcaggagtgctgtttataccagatgaatctacaaagccaagaacaggaatcaccgtatctccttcagtagacctgatactgtgagtcttctcttttctgttgacatatttgt

SEQ ID NO: 13 - DNA, 496 bp, Homo sapiens
  misc_feature (126)..(128): n is a, c, g, or t
gcaaagcctttaagtttttccactggaattctaacctcaccattctgaaaaccaacataagtcacaatggcacctaccattgctcaggcatgggaaagcatcgctacacatcagcaggaatatcannntnnnctgtnnnaggcctccagttaccaactcctgtctggtttcatgtccttttctatctggcagtgggaataatgtttttagtgaacactgttctctgggtgacaatacgtaaagaactgaaaagaaagaaaaagtggaatttagaaatctctttggattctggtcatgagaagaaggtaatttccagccttcaagaagacagacatttagaagaagagctgaaatgtcaggaacaaaaagaagaacagctgcaggaaggggtgcaccggaaggagccccagggggccacgtagcagcggctcagttggtggccatcgatctggaccgtcccctgcccacttgctccccatgagcactgcgtacaaac

SEQ ID NO: 14 - DNA, 217 bp, Homo sapiens
gtggcagaaccctgcagggacatggaattaaggccattgcaatgtatcatctttgtagcattgtcatcactcctaagctgccttcacagttttagtacactaagatgaggaaatcgaaaatgggcagagaaagctcatactgtataattgaagacagtgacagagaacgtgtcagttatgccaaaactcttttgatttctgttccaggatttccaac

SEQ ID NO: 15 - DNA, 455 bp, Homo sapiens
tctacctgtcctttgagaactctcagcaccgcgactacattacggagaaattctggcgcaacgcactggtggctggcactgtgccagtggtgctggggcccccacgggccacctatgaggccttcgtgccggctgacgccttcgtgcatgtggatgactttggctcagcccgagagctggcggctttcctcactggcatgaatgagagccgataccaacgcttctttgcctggcgtgacaggctccgcgtgcgactgttcaccgactggcgggaacgtttctgtgccatctgtgaccgctacccacacctaccccgcagccaagtctatgaggaccttgagggttggtttcaggcctgagatccgctggccgggggaggtgggtgtgggtggaagggctgggtgtcgaaatcaaaccaccaggcatccggcccttaccggcaagcagcgggctaa

SEQ ID NO: 16 - DNA, 508 bp, Homo sapiens
gatcttaaagccacggagaagcctctcatcttatggcattgacaaagagaagaccatccaccttaccctgaaagtggtgaagcccagtgatgaggagctgcccttgtttcttgtggagtcaggtgatgaggcaaagaggcacctcctccaggtgcgaaggtccagctcagtggcacaagtgaaagcaatgatcgagactaagacgggtataatccctgagacccagattgtgacttgcaatggaaagagactggaagatgggaagatgatggcagattacggcatcagaaagggcaacttactcttcctggcatcttattgtattggagggtgaccaccctggggatggggtgttggcaggggtcaaaaagcttatttcttttaatctcttactcaacgaacacatcttctgatgatttcccaaaattaatgagaatgagatgagtagagtaagatttgggtgggatgggtaggatgaagtatattgcccaactctatgtttctttga

SEQ ID NO: 17 - DNA, 245 bp, Homo sapiens
ttctctctgtgtggcaaacatttaaaaaaaaaaaaaaaaagcaggaaagatcccaagtcaaacagtgtggcttaaacattttttgtttcttggtgttgttatggcaagtttttggtaatgatgattcaatcattttgggaaattcttgcactgtatccaagttatttgatctggtgcgtgtggccctgtgggagtccactttcctctctctctctctctctgttccaagtgtgtgtgcaatgttc

SEQ ID NO: 18 - DNA, 466 bp, Homo sapiens
  misc_feature (245)..(245): n is a, c, g, or t
gggagaactaggtgtccagatttttgtatgtgttgtttttcttgggggatggggtggggtgggagtaggtagagctgagaatactacatcttagtggtgacctttagccacgtgggtgaagtggcaaaggccatggccatatctgttgtcccaggccaaagactaacaactgccttgggaatcccttccttgtgtccttaccaaatgatagctcataaaactctgataatgtaacaaatcacttncaaaggagttcccagaagtcttcagaaagactaaaattctgtctcttcctgctttagacagccattaagatcccaactaattttaccgaacctaaaacccacaaagaggttgtttgtgttattgttcaatcttcagttgtaagagtaattctctatttttatattgaaacataattacttgatagctcagggtctacacttcattcaactttttacaccaa

SEQ ID NO: 19 - DNA, 164 bp, Homo sapiens
atgcatacgggtgaatgtttgtgtgcccatgagtatatgcatatgggtatatgtttgtgtgcccgtgattatatgcatatgggtgaatgtttgtgtgcccgtgattatatgcatgtgggtgaatgttcatgaacacaaacagctacactcggagtagaaaacaa

SEQ ID NO: 20 - DNA, 481 bp, Homo sapiens
atctgccaaagcccagccaattaaaggagccagaagagctccggattcttcatcggaatttgggaaggtgactcgaatgtgttttgctgtgttcctctgctttgccctgagctacatccccttcttgctgctcaacattctggatgccagagtccaggctccccgggtggtccacatgcttgctgccaacctcacctggctcaatggttgcatcaaccctgtgctctatgcagccatgaaccgccaattccgccaagcatatggctccattttaaaaagagggccccggagtttccataggctccattagaactgtgaccctagtcaccagaattcaggactgtctcctccaggaccaaagtggccaggtaataggagaataggtgaaataacacatgtgggcattttcacaacaatctctccccagcctcccaaatcaagtctctccatcacttgatcaatgtttcagccctagactgcc

SEQ ID NO: 21 - DNA, 546 bp, Homo sapiens
cttcactgtgtggacctagagactccaacttgaattcctagtaactttcttggtatgctggccagaaagggaaatgaggaggagagtagaaaccacagctcttagtagtaatggcatacagtctagaggaccattcatgcaatgactatttctaaagcacctgctacacagcaggctgtacacagcagatcagtactgttcaacagaacttcctgagatgatggaaatgttctacctctgcactcactgtccagtacattagacactaggcacattggctgttaatcacttggaatgtgtttagcttgactgaggaattaaattttgattgtaaatttaaatcgccacacatggctagtggctactgtattggagtgcacagctctagatggctcctagattattgagagcctccaaaacaaatcaacctagttctatagatgaagacataaaagacactggtaaacaccaatgtaaaagggcccccaaggtggtcatgactggtctcatttgcagaagtctaagaatgtacctttttctggccgg

SEQ ID NO: 22 - DNA, 263 bp, Homo sapiens
  misc_feature (28)..(28): n is a, c, g, or t
catgtaagaattcccgggagctccatgncnttttaattttagccattctnctgncctcatttcttaaaattagaganttaaggtcccgaaggtggaacatgcttcatggtcacacatacaggcacaaaaacagcattatgtggacgcctcatgtattttttatagagtcaactatttcctctttattttccctcattgaaagatgcaaaacagctctctattgtgtacagaaagggtaaataatgcaaaatacctggtagtaa

SEQ ID NO: 23 - DNA, 496 bp, Homo sapiens
ttcaaattttctgcctgagggaagccatctcagcaataataatgaaattacttccttaacacttaaaccaccagttgattccttagactcaggaaataatcccaggttacaaaagcatcctaattttgctttttctgtttcaagtgtgaattcactaagcaacacaatatttcttggagaattaagcctcatattaaatcaaggagaatgcagttctcctgacatacaaaactcagtagaggaggaaaccaccatgcttttggaaaatgattcacccagtgaaactattccagaacagaccctgcttcctgatgaatttgtctcctgtttggggatcgtgaatgaggagttgccatctattaatacttattttccacaaaatattttggaaagccacttcaataggatttcactcttggaaaagtagagctgtgtggtcaaaatcaatatgagaaagctgccttgcaatctgaacttgggttttccctgcaataga

SEQ ID NO: 24 - DNA, 477 bp, Homo sapiens
attgtggtgagcgatcctgactccacagatgcttcaagcattgaagacaatgaggacatttgtaacaccacctccttggagaattgcacagcaaaatgagcgggggtgtttgtgcctgtttctcttatcctttcccaacattaggttaacacagctttataaacctcagtgggttcgttaaaatcatttaattctcagggtgtacctttccagccatagttggacattcattgctgaattctgaaatgatagaattgtctttatttttctctgtgaggtcaattaaatgccttgttctgaaatttattttttacaagagagagttgtgatatagtttggaatataagataaatggtattgggtggggtttgtggctacagcttatgcatcattctgtgtttgtcatttactcacattgagctaactttaaattactgacaagtagaatcaaaggtgcagctgactgagacgacatgc

SEQ ID NO: 25 - DNA, 371 bp, Homo sapiens
tgggatatcagtgaactatgttgtatacttttgaatttttacattttataaatggaattgaaagttggataactgctttttttaaattttccaacagaagtaacaccacagttgctttgtttctttttatagcttacctgaggttcagttcttctttgtgaacctgtgagtactccacagtttactgggggaaaaggcttcagtaaagcagaggctagaattacagtatttatacatagcaacttttcataaagtagaaaaattcaaaggaagctgtctcaatttgagaataccagctgggcacggtggctcacgcctgtaatcccagcacttactttgggaggccaaggtgggcagataacctgcggtca

SEQ ID NO: 26 - DNA, 201 bp, Homo sapiens
  misc_feature (172)..(172): n is a, c, g, or t
tctctgagcggacccggtgggaatccgagcggctccctcgggagcggggactgctggaacagcggctattcttctgttattagtatttttcttactgtccaagatcaactgtggaaatattttggtctcttgtgacgttcggtgcagtttcgttgtgaagtttgctattganatngaaattatgacttctgtgtttcctga

SEQ ID NO: 27 - DNA, 442 bp, Homo sapiens
  misc_feature (75)..(75): n is a, c, g, or t
ttataactgccatctccaactacatccttatgatgtttttaacaataaaattaaaacaactgttaaactaaaaanccacaccgttttnccagtacttgatctctgagctacaatactcnnctaaatataattttccaatcaaaatattctattctatattctaagggttaatatgtgattatagtgtccacttgccaccatttttttaaatcaatggacttgaaaagtattaatttagatggatgcgcagatataccctcagttcagtcatagattggagtttgcatataataatgtaaatgtatgtcgacactattctaaatagttctattatgactgaaatttaattaaataaaaaaggttgtaaaatgtgatgtgtatgtgtatatactgtatgtgtactttttaaaataggtgtatgtcccaacccttttttatacag

SEQ ID NO: 28 - DNA, 472 bp, Homo sapiens
acatacagacatatgcatccccacacacgcctatgcacaaacgtggattatcgcacagactgggaggtttagtggtgcatttctcctctgttttctttttaatatacatttaaaatacagtattatcactttataaaacatacattaagcctaataaatggaccaataagccaaactatcagtattttgtatatcctgcataaactctaatttagttcctcaacatattttcagtgtttatgcagacctttagagttaagcctttgtatttccatgttattccacaatatgcaatatttctctgagtagcttctgctatgatattcttatgaagaaaaggggcaactttctgtccactataggagagaattcagccgaagatatgagagtaatgagagacattttccagtcattggatcgtgttttcttttgtccattattgtactgtgctgtaccacatttatttctatat

SEQ ID NO: 29 - DNA, 444 bp, Homo sapiens
agcccacctgctggatgaggaacttgaggcaagtcaccagcccctgatcatttcgcctaaaagagcaaggactagagttcctgacctccaggccagtccctgatccctgacctaatgttatcgcggaatgatgatatatgtatctacgggggcctggggctgggcgggctcctgcttctggcagtggtccttctgtccgcctgcctgtgttggctgcatcgaagagcaccttctgtcctggtcccaggcccagggctcctcagagcaggaactccactatgcatctctgcagaggctgccagtgcccagcagtgagggacctgacctcaggggcagagacaagagaggcaccaaggaggatccaagagctgactatgcctgcattgctgagaacaaacccacctgagcaccccagacaccttcctcaacccaggcgggtggaca

SEQ ID NO: 30 - DNA, 535 bp, Homo sapiens
  misc_feature (45)..(81): n is a, c, g, or t
cgggcaccatgataccctatgttctggggattagcagtgaggaannnnnnnnnnnnnnnnnnnnnnnnnnnnnnnnnnnnngatgaggaacttgaggcaagtcaccagcccctgatcatttcgcctaaaagagcaaggactagagttnctgnnntncaggccagtccctgatccctgacctaatgttatcgcggaatgatgnnnngcccagggctcctcagagcaggaactccactatgcatctctgcagaggctgccagtgcccagcagtgagggacctgacctcaggggcagagacaagagaggcaccaaggaggatccaagagctgactatgcctgcattgctgagaacaaacccacctgagcaccccagacaccttcctcaacccaggcgggtggacagggtccccctgtggtccagccagtaaaaaccatggtccccccacttctgtgtctcagtcctctcagtccatctcgagcctccgttcaaantgatcatcatcaaaacttatgtggctttttgacctttgaatag

SEQ ID NO: 31 - DNA, 431 bp, Homo sapiens
  misc_feature (131)..(131): n is a, c, g, or t
aagcagcctagggatgagcatttctttgaaagcaattaggttattcacctggtattaaaactatttactgttaaaaaatctgtgacttcatgaagttgatttttaaaggcagcatcaaaaactgaaaaggnaagggaaaaaataggcagcttctctgcacttgtttggagctccccaaaacaggagccatggagaagtggcatcaagaccgggctgccctttcgagaacaccctgtggcagttcagagacacgcttttcctacactgcatgcagcccctctttccagcactggaaagaagtggtcttgagcccagctgagaagcacttcacactcctctctcttgttctgaatggtgtttgtgtcagtctgcagctgtgtatggtattatgtcttataatcctgcatcacttctatcctatccagtcatat

SEQ ID NO: 32 - DNA, 563 bp, Homo sapiens
  misc_feature (35)..(35): n is a, c, g, or t
aatggcttctatgatcagaactgggaaaacagtgnatcttatggtggaagaggtnctcagcaagtgtacagtatttaccttcctttgtcttacatnggctttttaaattttccattaatttcaacataattatgggaacaagtgtacagaagaattttttttttaagatatgtgagaacttttcatagatgaactttttaacaaatgttttcatttacaggaaattgcaaagaaaattctcaagtgatagtctttttttttaagtgtttcgtaagacaaaaattgaataatgttttttgaagttctggcaagattgaagtctgatattgcagtaatgatatttattaaaaacccataactaccaggaataatgatacctcccaccccttgattcccataacataaaagtgctacttgagagtgggggagaatggcatggtaggctacttttcagggccttgacaagtacatcacccagtggtatcctacatacttctttcaagatcttcaaccatgaggtaaaagagccaagttcaaagaaccctagcacaaatttgctttgg

SEQ ID NO: 33 - DNA, 218 bp, Homo sapiens
tcacttccctaatttccctaagtagtcctcacccatcactctttccatcttttggccctgccgcttcctcacttacagtttttctttcctgtcttaactagaagagaccttcgacttaaaggtaaccactttccctctgagatgctgtataaaactggaggttaggggatatttatggttgctggtgataactttgttgttctccaggcaaaaacgtc

SEQ ID NO: 34 - DNA, 535 bp, Homo sapiens
  misc_feature (256)..(256): n is a, c, g, or t
tagagatcatctagtcccatcaactcactatatatatgaggaacctgaggtccagagtggggaagtgtcttacccaaggtcacatggtttcagagaaattatgttgaatccaataagccttcccggacattccaagcctcttaaccatggcatctatgttgaggatgtcaatgtttatttcagcaaaggacgtcatggcttttaaaaactccttttaagcctccttgttttgatgtcaccttggtaggctgggccntctgagaggttggaagctctaggcattgttctctttggatccagggatgctaagtagaaactgcatgagccaccagtgccccggcaccctttaacaccaccagatgggtgttttcccccatccaccactggcagggttgccccttccctccaatcatcactgtgctccttttttcccggcctacgaggcagctcctgccactatctttagagccaataaagagaattaaaaacctgtgcaccaggnagcatcttttaaatacactagccattctcttgc

SEQ ID NO: 35 - DNA, 377 bp, Homo sapiens
ttgaaagttgtgggtcagctgaccaggtagaggattcaagactcaatgtggaaaaaatattttaaactactgattgaatgttaatggtcaatgctagcacaatattcctatgctgcaatacattaaaataactaagcaagtatatttatttctagcaaacagatgtttgttttcaaaatacttctttttcattattggttttaaaaaagcattatccttttatctcacaaataagtaatatctttcagttattaaatgatagataatgcctttttggttttgtgtggtattcaactaatacatggtttaaagtcacagccgtttgaatatattttatcttggtagtacattttctcccttaggaatatacatagtct

SEQ ID NO: 36 - DNA, 488 bp, Homo sapiens
gatctctgattcattgtccattttactaccaaatattaactaaggccttattaatttttatataaattatatcttgtcctattaaatctagttacaatttatttcatgcataagagctaatgttattttgcaaatgccatatattcaaaaaagctcaaagataattttctttactattatgttcaaataatattcaatatgcatattatctttaaaaagttaaatgtttttttaatcttcaagaaatcatgctacacttaacttctcctagaagctaatctataccataatattttcatattcacaagatattaaattaccaattttcaaattattgttagtaaagaacaaaatgattctctcccaaagaaagacacattttaaatactccttcactctaaaactctggtattataacttttgaaagttaatatttctacatgaaatgtttagctcttacactctatccttcctagaaaatggtaatt

SEQ ID NO: 37 - DNA, 457 bp, Homo sapiens
  misc_feature (292)..(292): n is a, c, g, or t
acttggcacccatgtctaccaaagtttacaattcaaaatgagaaaagagaggatgtactaaaaataagtggtccatgtgttgtgtgcagctgttgtggagatgttgattttgagattaaatctcttgatgaacagtgtgtggttggcaaaatttccaagcactggactggaattttgagagaggcatttacagacgctgataactttggaatccagttccctttagaccttgatgttaaaatgaaagctgtaatgattggtgcctgtttcctcattgacttcatgttttttngaaagcannnnnnnngccaggaacaaaaatcaggagtgtggtagtggattagtgaaagtctcctcaggaaatctgaagtctgtatattgattgagactatctaaactcatacctgtatgaattaagctgtaaggcctgtagctctggttgtatacttttgctttt

SEQ ID NO: 38 - DNA, 110 bp, Homo sapiens
gaaagagcgagagaaggggaaagacaagtcgggagaggccggtaggcgtgaggcgggcctgaagcggcagcgggcggccttcgtccggcgagagctaggccgaggacccg

SEQ ID NO: 39 - DNA, 438 bp, Homo sapiens
aatgggtgccggagattcgatgccactgtcccaaagcccccatcatcctagttggaacgcagtcggatctcagagaagatgtcaaagtcctcattgagttggacaaatgcaaagaaaagccagtgcctgaagaggcggctaagctgtgcgccgaggaaatcaaagccgcctcctacatcgagtgttcagccttgactcaaaaaaacctcaaagaggtctttgatgcagccatcgtcgctggcattcaatactcggacactcagcaacagccaaagaagtctaaaagcaggactccagataaaatgaaaaacctctccaagtcctggtggaagaagtactgctgtttcgtatgatgctggcaagacacccagaaaggctattttcagatgaaatcgatattagaagctatattagctgaaacaactccttttactgcgt

SEQ ID NO: 40 - DNA, 477 bp, Homo sapiens
  misc_feature (30)..(30): n is a, c, g, or t
tactgtactgtttcaatgtgttaagtgccntgntgtaaagtaaaattttaagtctagattcattattttcctgacatatatttttcattatgatatctactgtatgctattgtgatagttttatgaaatgcttacattttaatcaaatatgtaaatttcagaagctcttttttcctacccaccagtaccttaatcattggtttatcacattggattcaaattcaggttccttttttgataaagaggaaatttgttttccatgcttgtgattttgtgtttgtaaacatttcaggaggaatttaggagtgtaactatagtttatacttctataatgttgtcttacatttacatttttaagtgcctaattgttaaaactgatttatgtttctcttctaaagaggatatgtgtacttggtttctaatctctttggttttgctttttaaaaaatgcaagagcctatactttgtatttaccta

SEQ ID NO: 41 - DNA, 405 bp, Homo sapiens
  misc_feature (362)..(362): n is a, c, g, or t
gtggagaactggcagtgacctctactgggggccatggcagggaggggagccttctggaagggctgccttggagattggaatggggactcccagggagacctgcgttccatccctgcctgcctcacccctgccacagactctgcacaccactggatggtgggtccaagcctggcacagtccctgtgcttgtcagagtcattattatgattaatatcaattacgatgccaaaaattgctgggcaaactttgaagacctcaacttgttacaatgacgatgatgatgattcttggcggttacacaatccttcctcctgggggggaggcagctaggaggcccagcaggggggcttctatgctgctgngctcccctagggagttggggtagtctgtgccaactccaggcag

SEQ ID NO: 42 - DNA, 520 bp, Homo sapiens
aactttgtatagcccatgtacctaccttgtatagaaaaataattttaaaaatttgaatggaagggggtaaaggaagtcatgaagtttttttgcatttttatttaaatgaaggaattccaaataactcacctacagatttttagcacaaaaatagccattgtaaagtgttaaaatttacgataagtattctattggggaggaaaggtaactctgatctcagttacagtttttttttcctttttaatttcattattttgggtttttggtttttgcagtcctatttatctgcagtcgtattaagtcctattgctagaataggttactacaaaaaaggttatattctgaaagaaaaataactgacattatatataaccaattaatttaaagtattgccatttaaattacacactgagagcatgtcctatgcagacatagatttttctgttcatttatttttcttcattgcagtggattgatttgataaatagatgtgttgaattactacatttgctgtacatat

SEQ ID NO: 43 - DNA, 486 bp, Homo sapiens
ttcagcttcatttgtgtcaatgggcaatgacaggtaaattaagacatgcactatgaggaataattatttatttaataacaattgtttggggttgaaaattcaaaaagtgtttatttttcatattgtgccaatatgtattgtaaacatgtgttttaattccaatatgatgactcccttaaaatagaaataagtggttatttctcaacaaagcacagtgttaaatgaaattgtaaaacctgtcaatgatacagtccctaaagaaaaaaaatcattgctttgaagcagttgtgtcagctactgcggaaaaggaaggaaactcctgacagtcttgtgcttttcctatttgttttcatggtgaaaatgtactgagattttggtattacactgtatttgtatctctgaagcatgtttcatgttttgtgactatatagagatgtttttaaaagtttcaatgtgattctaatgtcttcatttcattgtatgatg

SEQ ID NO: 44 - DNA, 558 bp, Homo sapiens
  misc_feature (124)..(124): n is a, c, g, or t
tcatgatgctgactggcgttagctgattaacccatgtaaataggcacttaaatagaagcaggaaagggagacaaagactggcttctggacttcctccctgatccccacccttactcatcacctngcagtggccagaattagggaatcagaatcaaaccagtgtaaggcagtgctggctgccattgcctggtcacattgaaattggtggcttcattctagatgtagcttgtgcagatgtagcaggaaaataggaaaacctaccatctcagtgagcaccagctgcctcccaaaggaggggcagccgtgcttatatttttatggttacaatggcacaaaattattatcaacctaactaaaacattccttttctcttttttcctgaattatcatggagttttctaattctctcttttggaatgtangattttttttaaatgctttacgatgtaaaatatttattttttacttattctggaagatctggctgaaggattattcatggaacaggaagaagcgtaaagactatccatgtcatctttgttgagagtcttcgtgact

SEQ ID NO: 45 - DNA, 552 bp, Homo sapiens
aacttacatacaaatattacctcatttgttgtgtgactgagtaaagaatttttggatcaagcggaaagagtttaagtgtctaacaaacttaaagctactgtagtacctaaaaagtcagtgttgtacatagcataaaaactctgcagagaagtattcccaataaggaaatagcattgaaatgttaaatacaatttctgaaagttatgttttttttctatcatctggtataccattgctttatttttataaattattttctcattgccattggaatagaatattcagattgtgtagatatgctatttaaataatttatcaggaaatactgcctgtagagttagtatttctatttttatataatgtttgcacactgaattgaagaattgttggttttttcttttttttgttttttttttttttttttttttttttgcttttgacctcccatttttactatttgccaatacctttttctaggaatgtgcttttttttgtacacatttttatccattttacattctaaagcagtgtaagttgtatattactgtttct

SEQ ID NO: 46 - DNA, 379 bp, Homo sapiens
gttttcactgcttgtatgatgtttcccattcatacacctataaatctctaacaagaggccctttgaactgccttgtgttctgtgagaaacaaatatttacttagagtggaaggactgattgagaatgttccaatccaaatgaatgcatcacaacttacaatgctgctcattgttgtgagtactatgagattcaaatttttctaacatatggaaagccttttgtcctccaaagatgagtactagggatcatgtgtttaaaaaaagaaaggctacgatgactgggcaagaagaaagatgggaaactgaataaagcagttgatcagcatcattggaacatggggacgagtgacggcaggaggaccacgaggaaataccctca

SEQ ID NO: 47 - DNA, 519 bp, Homo sapiens
ttggctcttggtggaattgaattcatcttgtgtcttattcaagtaataaatggagtgcttggaggcatatgtggcttttgctgctctcaccaacagcaatatgactgctaaaagaaccaacccaggacagagccacaatcttcctctatttcattgtaatttatatatttcacttgtattcatttgtaaaactttgtattagtgtaacatactccccacagtctacttttacaaacgcctgtaaagactggcatcttcacaggatgtcagtgtttaaatttagtaaacttcttttttgtttgtttatttgtgtaacatactccccacagtctacttttacaaacgcctgtaaagactggcatcttcacaggatgtcagtgtttaaatttagtaaacttcttttttgtttgtttatttgtttttgtttttttttaaggaatgaggaaacaaaccaccctctgggggtagtttacagactgagtgacagtactcagtatatctgagataaactctataatg

SEQ ID NO: 48 - DNA, 178 bp, Homo sapiens
  misc_feature (119)..(119): n is a, c, g, or t
tatgactgctaaaagaaccaacccaggacagagccacaatcttcctctatttcattgtaatttatatatttcacttgtattcatttgtaaaactttgtattagtgtaacatactccccncagtntacttttacaaacgcctgtaaagactggcatcttcacaggatgtcagtgtttaa

SEQ ID NO: 49 - DNA, 496 bp, Homo sapiens
ctttctgcctgtactggatctgttattttcagggaaacaggccccagggcccccctgagcctcaccctaagcccttaggcctctgagagtgctgttgggttctatttatttatttatttgttcctttgttccctacccgtgcccccagtgtcttccctgctgagtaccaggagaggtcctgccccatcctctctctgaagccagggcccttccattccatttagcctttggatcatcctggctgggagaagtgggaccgagccacccagccccactatccccaagcagccctacagccgggatgggaggcacgtggcctctcttttatccgtctatttattttgtaagtgtattcgtgtggaggaggttgttgctttatttttttaaggctctggagtgttgtgtatggtttcttttcacatcccagcctcccatgggcacttctaagaagagaggggatttcttggaaaaggagagaggaatcccctagagcagg

SEQ ID NO: 50 - DNA, 527 bp, Homo sapiens
  misc_feature (446)..(446): n is a, c, g, or t
gagtggacgttgcacatcactgtgaaaccttgcagaggaggagagggcaggttcatcagaagaaagaaaggacaaaatgactccttatgaagcattttgtgccttctgtgagaaaacatgtatttagatcagataaactctagtcaaaatacaaaaaggaaaaatgaaagacctctgaaataggaacaatctcttgaagaggcaaatgactcaaaactgctcagtggctctttcagaaaatctaagtaaagttccctgacaacagaaactgaagagattgcctggttcatcttgtagtcttccaaaacagcagataatttctgaatctcagatgttgaatcagtacaacgggatggatttcttgttcctaagtgttaaatgatcacatacataaaagagtctgagcctgagcaacatagtagagaccctgtctctacaaaaaaaanaaaaaattagccaggcgtggtggtgcacactgatagtcccagctactcaggaggctgagggaggcggatcaattgagctag

SEQ ID NO: 51 - DNA, 356 bp, Homo sapiens
  misc_feature (58)..(58): n is a, c, g, or t
ccaggagccctggctagggtagtgatgacatgcacccaaacacccctaaacacaggtngcctcgctttgangggaccttgtaaagaaaatangccccaggccccagtggtgggaggcaggttgtctagaacacagccttgcctcaaaaagaaaaaaataaagaaaagaaaatctgaaagggaaagcaggcatcaacttccagggatctaataacttcctaacttaaagcaacagaacttacctaacttttggacttttcattttaaaactttgcctctagcagtngacactgtcctcaacacagctacagtttatgtaacacagagtccatattatatggggcatgtctgtatcaa

SEQ ID NO: 52 - DNA, 499 bp, Homo sapiens
gagacctggagatctaattctgtttagacattttttgtcccagaaatacagaagcttgaaatgctatgaaggcagagcttctattctttatgggatgaaatatttcaaaagaggataaatcctctgtggtaagccatttggaaaatcctaccaagaattggcttatttaattttccagaaccaggaatgagtatctaatagcttttgtagaaccttccagaatatgtggggaaaaagggctattgctaagtgagctttatctaatatcctcctaagagttttactagtgcttttttgaggaattacagggaagctcctggaattgtacatggatatctttatccctagggggaaatcaaggagctgggcacccctaattctttatggaagtgtttaaaactattttaattttattacaagtattactagagtagtggttctactctaagatttcaaaagtgcatttaaaatcatacatgttcccgcctgcaaatatatt

SEQ ID NO: 53 - DNA, 464 bp, Homo sapiens
  misc_feature (117)..(117): n is a, c, g, or t
gaaaaacaccatgcatctgatgagataattcataaatgttgccccttttttaaatgatacaaccctaaaagtgactgaattgcccaagtgcttgaacatggcagaggtagttactcntattttgcagtttgtgcacttaaaaattcctacagtgattgttactttactggggaaaaaagatgaggtgaaacttcctcccaaggaattaaaatatctgtagaagccatggcttgcttttataatgtggaaatcatttgatttgctgtaattcacgcagatccctccttttgtcagggggaaatgatttgcatcatgttcttttttcataatgcttttacttcctgtttggatcagttgtatgtaaatgtacatttttgttactttggctgtgcccgttagaatttatcttccataaagtatttctcccattgagtctaatgatgtatactttgcctaggtctttc

SEQ ID NO: 54 - DNA, 443 bp, Homo sapiens
ctttgctatgacttctggccagatgtggaaccatatccgtggacctccatatgctcataagaacccacacaatggacaagtgagctacattcatgggagcagccaggctcagtttgtggcagaatcacacattattctggtactgaatgccgctatcaccatggggatggttcttctaaatgaagcagcaacttcgaaaggcgatgttggaaaaagacggataatttgcctagtgggattgggcctggtggtcttcttcttcagttttctactttcaatatttcgttccaagtaccacggctatccttatagctttttaattaaatgaagccaagtgggatttgcataaagtgaatgtttaccatgaagataaactgttcctgactttatactattttgaattcattcatttcattgtgatcagctagcttattcttgtgtac

SEQ ID NO: 55 - DNA, 540 bp, Homo sapiens
tgagaatggacgctgcatccaggccaactactcactaatggaaaacggaaagatcaaagtgttaaaccaggagttgagagctgatggaactgtgaatcaaatcgaaggtgaagccaccccagttaacctcacagagcctgccaagctggaagttaagttttcctggtttatgccatcggcaccgtactggatcctggccaccgactatgagaactatgccctcgtgtattcctgtacctgcatcatccaactttttcacgtggattttgcttggatcttggcaagaaaccctaatctccctccagaaacagtggactctctaaaaaatatcctgacttctaataacattgatgtcaagaaaatgacggtcacagaccaggtgaactgccccaagctctcgtaaccaggttctacagggaggctgcacccactccatgttacttctgcttcgctttcccctaccccacccccccccataaagacaaaccaatcaaccacgacaaaggaagttgacctaaacatgtaaccatgccctaccct

SEQ ID NO: 56 - DNA, 566 bp, Homo sapiens
tctatacctcctggcagattccacaaaagttcatagttgactattctgaaaccagcccccagtgccccaagccaggtgtcatcctcctaaccaagagaggccggcagatctgtgctgaccccaataagaagtgggtccagaaatacatcagcgacctgaagctgaatgcctgaggggcctggaagctgcgagggcccagtgaacttggtgggcccaggagggaacaggagcctgagccagggcaatggccctgccaccctggaggccacctcttctaagagtcccatctgctatgcccagccacattaactaactttaatcttagtttatgcatcatatttcattttgaaattgatttctattgttgagctgcattatgaaattagtattttctctgacatctcatgacattgtctttatcatcctttcccctttcccttcaactcttcgtacattcaatgcatggatcaatcagtgtgattagctttctcagcagacattgtgccatatgtatcaaatgacaaatctttattgaatggttttgctcagcaccaccttttaatata

SEQ ID NO: 57 - DNA, 519 bp, Homo sapiens
  misc_feature (362)..(389): n is a, c, g, or t
atagttgactattctgaaaccagcccccagtgccccaagccaggtgtcatcctcctaaccaagagaggccggcagatctgtgctgaccccaataagaagtgggtccagaaatacatcagcgacctgaagctgaatgcctgaggggcctggaagctgcgagggcccagtgaacttggtgggcccaggagggaacaggagcctgagccagggcaatggccctgccaccctggaggccacctcttctaagagtcccatctgctatgcccagccacattaactaactttaatcttagtttatgcatcatatttcattttgaaattgatttctattgttgagctgcattatgaaattagtattttcnnnnnnnnnnnnnnnnnnnnnnnnnnnnatcctttcccctttcccttcaactcttcgtacattcaatgcatggatcaatcagtgtgattagctttctcagcagacattgtgccatatgtatcaaatgacaaatctttattgaatggttttgctcagca

SEQ ID NO: 58 - DNA, 475 bp, Homo sapiens
ctcgctcagccagatgcaatcaatgccccagtcacctgctgctataacttcaccaataggaagatctcagtgcagaggctcgcgagctatagaagaatcaccagcagcaagtgtcccaaagaagctgtgatcttcaagaccattgtggccaaggagatctgtgctgaccccaagcagaagtgggttcaggattccatggaccacctggacaagcaaacccaaactccgaagacttgaacactcactccacaacccaagaatctgcagctaacttattttcccctagctttccccagacatcctgttttattttattataatgaattttgtttgttgatgtgaaacattatgccttaagtaatgttaattcttatttaagttattgatgttttaagtttatctttcatggtactagtgttttttagatacagagacttggggaaattgcttttcctcttgaaccacagttctaccc

SEQ ID NO: 59 - DNA, 361 bp, Homo sapiens
gcacgccatcaatgccatcgtgcgcagcccgcgtggcggggcccggaggcacgcagagctgtccttcgtgttcctcacggacggcgtcacgggcaacgacagtctgcacgagtcggcgcactccatgcgcaacgagaacgtggtacccaccgtcctggccttgggcagcgacgtggacatggacgtgctcaccacgctcagcctgggtgaccgcgccgccgtgttccacgagaaggactatgacagcctggcgcaacccggcttcttcgaccgcttcatccgctggatctgctagcgccgccgcccgggccccgcagtcgagggtcgtgagcccaccccgtccatggtgctaagcgggccc

SEQ ID NO: 60 - DNA, 287 bp, Homo sapiens
ccctgggagcatgtgaatgccatccaggaggcccggcgtctcctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggccagccactacaaacagcactgccctccaaccccggaaacttcctgtgcaacccagattatcacctttgaaagtttcaa

SEQ ID NO: 61 - DNA, 544 bp, Homo sapiens
taactctaccctggcactataatgtaagctctactgaggtgctatgttcttagtggatgttctgaccctgcttcaaatatttccctcacctttcccatcttccaagggtactaaggaatctttctgctttggggtttatcagaattctcagaatctcaaataactaaaaggtatgcaatcaaatctgctttttaaagaatgctctttacttcatggacttccactgccatcctcccaaggggcccaaattctttcagtggctacctacatacaattccaaacacatacaggaaggtagaaatatctgaaaatgtatgtgtaagtattcttatttaatgaaagactgtacaaagtataagtcttagatgtatatatttcctatattgttttcagtgtacatggaataacatgtaattaagtactatgtatcaatgagtaacaggaaaattttaaaaatacagatagatatatgctctgcatgttacataagataaatgtgctgaatggttttcaaataaaaatgaggtactctcctggaaatatt

SEQ ID NO: 62 - DNA, 399 bp, Homo sapiens
ttctttccccaaatatcatgtagcacatcaatatgtagggaaacattcttatgcatcatttggtttgttttataaccaattcattaaatgtaattcataaaatgtactatgaaaaaaattatacgctatgggatactggcaacagtgcacatatttcataaccaaattagcagcaccggtcttaatttgatgtttttcaacttttattcattgagatgttttgaagcaattaggatatgtgtgtttactgtactttttgttttgatccgtttgtataaatgatagcaatatcttggacacatttgaaatacaaaatgtttttgtctaccaaagaaaaatgttgaaaaataagcaaatgtatacctagcaatcacttttactttttgtaattctgtctct

SEQ ID NO: 63 - DNA, 416 bp, Homo sapiens
gagacttttctatggttttgtgactttcaacttttgtacagttatgtgaaggatgaaaggtgggtgaaaggaccaaaaacagaaatacagtcttcctgaatgaatgacaatcagaattccactgcccaaaggagtccaacaattaaatggatttctaggaaaagctaccttaagaaaggctggttaccatcggagtttacaaagtgctttcacgttcttacttgttgtattatacattcatgcatttctaggctagagaaccttctagatttgatgcttacaactattctgttgtgactatgagaacatttctgtctctagaagttatctgtctgtattgatctttatgctatattactatctgtggttacagtggagacattgacattattactggagtcaagcccttataagtc

SEQ ID NO: 64 - DNA, 367 bp, Homo sapiens
gattatcaattaccacaccatctcccatgaagaaagggaacggtgaagtactaagcgctagaggaagcagccaagtcggttagtggaagcatgattggtgcccagttagcctctgcaggatgtggaaacctccttccaggggaggttcagtgaattgtgtaggagaggttgtctgtggccagaatttaaacctatactcactttcccaaattgaatcactgctcacactgctgatgatttagagtgctgtccggtggagatcccacccgaacgtcttatctaatcatgaaactccctagttccttcatgtaacttccctgaaaaatctaagtgtttcataaatttgagagtctgtgacccacttacc

SEQ ID NO: 65 - DNA, 515 bp, Homo sapiens
  misc_feature (149)..(150): n is a, c, g, or t
gggggactgctttcaaatcagccaaagagcctgtcagatcttcttagaaggaagaggttggtagttccctgctctgttttgaacatgctctagtttattaacctggggacattcccattgctgtcttaagtaagtctcatagccagctnntgtcacgtgactctcatatggattcattttcgggccagctctgaacaaagcatcatgaacatatgtgcttttggtcgtttgcaatgtgatggtggtggaggtaggtattggtttccttggaaggcatgataagaaagattcacaatggccaacagtgtgtatgaacaaaaaactgattggagcatcagctagtactgaaggtccttgctttgtgtcagaggcaaaggaacccaaggcgccaagtcctcagccttgagtgtactgctgacaactaaactcacaggcngcaaagcagacctctgatgaagatgcctgttatttcacatcactgtctttttgtgtatcatagtctgcaccttacaaat

SEQ ID NO: 66 - DNA, 494 bp, Homo sapiens
tacacggccctgatgtcggctaagtactccggctggtacgtgggcttcaccaagaaggggcggccgcggaagggccccaagacccgggagaaccagcaggacgtgcatttcatgaagcgctaccccaaggggcagccggagcttcagaagcccttcaagtacacgacggtgaccaagaggtcccgtcggatccggcccacacaccctgcctaggccaccccgccgcggcccctcaggtcgccctggccacactcacactcccagaaaactgcatcagaggaatatttttacatgaaaaataaggaagaagctctatttttgtacattgtgtttaaaagaagacaaaaactgaaccaaaactcttggggggaggggtgataaggattttattgttgacttgaaacccccgatgacaaaagactcacgcaaagggactgtagtcaacccacaggtgcttgtctctctctaggaacagacaactctaaactcgtccc

SEQ ID NO: 67 - DNA, 497 bp, Homo sapiens
tacacggccctgatgtcggctaagtactccggctggtacgtgggcttcaccaagaaggggcggccgcggaagggccccaagacccgggagaaccagcaggacgtgcatttcatgaagcgctaccccaaggggcagccggagcttcagaagcccttcaagtacacgacggtgaccaagaggtcccgtcggatccggcccacacaccctgcctaggccaccccgccgcggcccctcaggtcgccctggccacactcacactcccagaaaactgcatcagaggaatatttttacatgaaaaataaggaagaagctctatttttgtacattgtgtttaaaagaagacaaaaactgaaccaaaactcttggggggaggggtgataaggattttattgttgacttgaaacccccgatgacaaaagactcacgcaaagggactgtagtcaacccacaggtgcttgtctctctctaggaacagacaactttaaactcgtccccag

SEQ ID NO: 68 - DNA, 521 bp, Homo sapiens
atatcttcttcaggctctgacaggcctcctggaaacttccacatatttttcaactgcagtataaagtcagaaaataaagttaacataactttcactaacacacacatatgtagatttcacaaaatccacctataattggtcaaagtggttgagaatatattttttagtaattgcatgcaaaatttttctagcttccatcctttctccctcgtttcttctttttttgggggagctggtaactgatgaaatcttttcccaccttttctcttcaggaaatataagtggttttgtttggttaacgtgatacattctgtatgaatgaaacattggagggaaacatctactgaatttctgtaatttaaaatattttgctgctagttaactatgaacagatagaagaatcttacagatgctgctataaataagtagaaaatataaatttcatcactaaaatatgctattttaaaatctatttcctatattgtatttctaatcagatgtattactcttattatttctat

SEQ ID NO: 69 - DNA, 464 bp, Homo sapiens
atgaactttgacatccctgaggagattaagcagctgcagcagttccagaaggaggacgccgcattgaccatctatgagatgctccagaacatctttgctattttcagacaagattcatctagcactggctggaatgagactattgttgagaacctcctggctaatgtctatcatcagataaaccatctgaagacagtcctggaagaaaaactggagaaagaagattttaccaggggaaaactcatgagcagtctgcacctgaaaagatattatgggaggattctgcattacctgaaggccaaggagtacagtcactgtgcctggaccatagtcagagtggaaatcctaaggaacttttacttcattaacagacttacaggttacctccgaaactgaagatctcctagcctgtccctctgggactggacaattgcttcaagcattcttcaaccagcagatgctgt

SEQ ID NO: 70 - DNA, 464 bp, Homo sapiens
accaggatgctcacatttaagttttacatgcccaagaaggccacagaactgaaacagcttcagtgtctagaagaagaactcaaacctctggaggaagtgctgaatttagctcaaagcaaaaactttcacttaagacccagggacttaatcagcaatatcaacgtaatagttctggaactaaagggatctgaaacaacattcatgtgtgaatatgcagatgagacagcaaccattgtagaatttctgaacagatggattaccttttgtcaaagcatcatctcaacactaacttgataattaagtgcttcccacttaaaacatatcaggccttctatttatttatttaaatatttaaattttatatttattgttgaatgtatggttgctacctattgtaactattattcttaatcttaaaactataaatatggatcttttatgattctttttgtaagccctagggg

SEQ ID NO: 71 - DNA, 216 bp, Homo sapiens
gtgtgaaggtgcagttttgccaaggagtgctaaagaacttagatgtcagtgcataaagacatactccaaacctttccaccccaaatttatcaaagaactgagagtgattgagagtggaccacactgcgccaacacagaaattattgtaaagctttctgatggaagagagctctgtctggaccccaaggaaaactgggtgcagagggttgtggagaa

SEQ ID NO: 72 - DNA, 441 bp, Homo sapiens
aaaggagcagtcgcacagacctttcctcatgctgcaggcccggcagtctgaagaccaccctcatcgccggcgtcggcggggcttggagtgtgatggcaaggtcaacatctgctgtaagaaacagttctttgtcagtttcaaggacatcggctggaatgactggatcattgctccctctggctatcatgccaactactgcgagggtgagtgcccgagccatatagcaggcacgtccgggtcctcactgtccttccactcaacagtcatcaaccactaccgcatgcggggccatagcccctttgccaacctcaaatcgtgctgtgtgcccaccaagctgagacccatgtccatgttgtactatgatgatggtcaaaacatcatcaaaaaggacattcagaacatgatcgtggaggagtgtgggtgctcatagagttgcccagc

SEQ ID NO: 73 - DNA, 425 bp, Homo sapiens
ctccgcggtcgccaaaaagaaagataaggtgaagaagggcggcccggggagcgagtgcgctgagtgggcctgggggccctgcacccccagcagcaaggattgcggcgtgggtttccgcgagggcacctgcggggcccagacccagcgcatccggtgcagggtgccctgcaactggaagaaggagtttggagccgactgcaagtacaagtttgagaactggggtgcgtgtgatgggggcacaggcaccaaagtccgccaaggcaccctgaagaaggcgcgctacaatgctcagtgccaggagaccatccgcgtcaccaagccctgcacccccaagaccaaagcaaaggccaaagccaagaaagggaagggaaaggactagacgccaagcctggatgccaaggagcccctggtgtcacatggggcct

SEQ ID NO: 74 - DNA, 510 bp, Homo sapiens
agatgatggaccctggttatcccaaactgattaccaagaacttccaaggaatcgggcctaaaattgatgcagtcttctattctaaaaacaaatactactatttcttccaaggatctaaccaatttgaatatgacttcctactccaacgtatcaccaaaacactgaaaagcaatagctggtttggttgttagaaatggtgtaattaatggtttttgttagttcacttcagcttaataagtatttattgcatatttgctatgtcctcagtgtaccactacttagagatatgtatcataaaaataaaatctgtaaaccataggtaatgattatataaaatacataatatttttcaattttgaaaactctaattgtccattcttgcttgactctactattaagtttgaaaatagttaccttcaaagcaagataattctatttgaagcatgctctgtaagttgcttcctaacatccttggactgagaaattatacttacttctggcataactaaa

SEQ ID NO: 75 - DNA, 360 bp, Homo sapiens
  misc_feature (99)..(99): n is a, c, g, or t
ctggcacataggcactgaaacactgcatgttaatccacaccccaccccacctatgagtgtagtcaaagctggtaagtgacaagggctttcgtggaaacntnncctgacctaatgttgggcatcaggttacccaaagagcttcaggnaaatgangaaaggacttgcaggtcttgantgagaatggagnggtaactgccaatgagggctttggctttagcngaaagtctgaaagggaagccataggaacttaaacgtaccgactataaagctctgagaaaagctgatgttttagaaagaccatacattctaggtacaaatacctaaaaactaaaaaataagtacgttggccaggcgggcgga

SEQ ID NO: 76 - DNA, 436 bp, Homo sapiens
tagtgtattcacttaccctagttcattataaaaaatatctaggcattgtggatataaaactgttgggtattctacaacttcaatggaaattattacaagcagattaatccctctttttgtgacacaagtacaatctaaaagttatattggaaaacatggaaatattaaaattttacacttttactagctaaaacataatcacaaagctttatcgtgttgtataaaaaaattaacaatataatggcaataggtagagatacaacaaatgaatataacactataacacttcatattttccaaatcttaatttggatttaaggaagaaatcaataaatataaaatataagcacatatttattatatatctaaggtatacaaatctgtctacatgaagtttacagattggtaaatatcacctgctcaacatgtaattatt

SEQ ID NO: 77 - DNA, 497 bp, Homo sapiens
cctgtcctccagcaccatagagaggcgctagagcccattcctctttctccaccgtcacccaacatcaatcctttaccactctaccaaataatttcatattcaagcttcagaagctagtgaccatcttcataatttgctggagaagtgtatttcttccccttactctcacacctgggcaaactttcttcagtgtttttcatttcttacgttctttcacttcaagggagaatatagaagcatttgatattatctacaaacactgcagaacagcatcatgtcataaacgattctgagccattcacactttttatttaattaaatgtatttaattaaatctcaaatttattttaatgtaaagaacttaaattatgttttaaacacatgccttaaatttgtttaattaaatttaactctggtttctaccagctcatacaaaataaatggtttctgaaaatgtttaagtattaacttacaaggatataggtttttctcatgta

SEQ ID NO: 78 - DNA, 445 bp, Homo sapiens
gagctcgattcacggaggcattgaaattttcagcagagaccttccaaggacatattgcaggattctgtaatagtgaacatatggaaagtattagaaatatttattgtctgtaaatactgtaaatgcattggaataaaactgtctcccccattgctctatgaaactgcacattggtcattgtgaatatttttttttttgccaaggctaatccaattattattatcacatttaccataatttattttgtccattgatgtatttattttgtaaatgtatcttggtgctgctgaatttctatattttttgtaacataatgcactttagatatacatatcaagtatgttgataaatgacacaatgaagtgtctctattttgtggttgattttaatgaatgcctaaatataattatccaaattgattttcctttgtgcatgtaaaaataac

SEQ ID NO: 79 - DNA, 252 bp, Homo sapiens
aaaacttcacatttatgtggcttgtttatccttagctcacagattgaggtaataatgacactcctagactttgggatcaaataacttagggccaagtcttgggtctgaatttatttaagttcacaacctagggcaagttactctgcctttctaagactcacttacatcttctgtgaaatataattgtaccaacctcatagagtttggtgtcaactaaatgagattatatgtggactaaatatctgtcatata

SEQ ID NO: 80 - DNA, 513 bp, Homo sapiens
gacgaccagccaggatatgctctcaatcatggagaaattggaattcttcgatttttcttatgaccttaacctgtgtgggctgacagaggacccagatcttcaggtttctgcgatgcagcaccagacagtgctggaactgacagagactggggtggaggcggctgcagcctccgccatctctgtggcccgcaccctgctggtctttgaagtgcagcagcccttcctcttcgtgctctgggaccagcagcacaagttccctgtcttcatggggcgagtatatgaccccagggcctgagacctgcaggatcaggttagggcgagcgctacctctccagcctcagctctcagttgcagccctgctgctgcctgcctggacttgcccctgccacctcctgcctcaggtgtccgctatccaccaaaagggctcctgagggtctgggcaagggacctgcttctattagcccttctccatggccctgccatgctctccaaaccactttttgcagctttctc

SEQ ID NO: 81 - DNA, 416 bp, Homo sapiens
gttggttcaaacttttgggagcacggactgtcagttctctgggaagtggtcagcgcatcctgcagggcttctcctcctctgtcttttggagaaccagggctcttctcaggggctctagggactgccaggctgtttcagccaggaaggccaaaatcaagagtgagatgtagaaagttgtaaaatagaaaaagtggagttggtgaatcggttgttctttcctcacatttggatgattgtcataaggtttttagcatgttcctccttttcttcaccctcccctttgttcttctattaatcaagagaaacttcaaagttaatgggatggtcggatctcacaggctgagaactcgttcacctccaagcatttcatgaaaaagctgcttcttattaatcatacaaactctcaccatgatgtg

SEQ ID NO: 82 - DNA, 526 bp, Homo sapiens
gtccacattcctgcaagcattgattgagacatttgcacaatctaaaatgtaagcaaagtagtcattaaaaatacaccctctacttgggctttatactgcatacaaatttactcatgagccttcctttgaggaaggatgtggatctccaaataaagatttagtgtttattttgagctctgcatcttaacaagatgatctgaacacctctcctttgtatcaataaatagccctgttattctgaagtgagaggaccaagtatagtaaaatgctgacatctaaaactaaataaatagaaaacaccaggccagaactatagtcatactcacacaaagggagaaatttaaactcgaaccaagcaaaaggcttcacggaaatagcatggaaaaacaatgcttccagtggccacttcctaaggaggaacaaccccgtctgatctcagaattggcaccacgtgagcttgctaagtgataatatctgtttctactacggatttaggcaacaggacctgtacattgtcacattgcat

SEQ ID NO: 83 - DNA, 380 bp, Homo sapiens
tactgcttgcagtaattcaactggaaattaaaaaaaaaaaactagactccattgtgccttactaaatatgggaatgtctaacttaaatagctttgagatttcagctatgctagaggcttttattagaaagccatatttttttctgtaaaagttactaatatatctgtaacactattacagtattgctatttatattcattcagatataagatttgtacatattatcatcctataaagaaacggtatgacttaattttagaaagaaaattatattctgtttattatgacaaatgaaagagaaaatatatatttttaatggaaagtttgtagcatttttctaataggtactgccatatttttctgtgtggagtatttttata

SEQ ID NO: 84 - DNA, 406 bp, Homo sapiens
gataaaacggcaacacagctcacaagaacagactttccagctgctgaagttatggaaacatcaaaacaaagcccaagatatagtcaagaagatcatccaagatattgacctctgtgaaaacagcgtgcagcggcacattggacatgctaacctcaccttcgagcagcttcgtagcttgatggaaagcttaccgggaaagaaagtgggagcagaagacattgaaaaaacaataaaggcatgcaaacccagtgaccagatcctgaagctgctcagtttgtggcgaataaaaaatggcgaccaagacaccttgaagggcctaatgcacgcactaaagcactcaaagacgtaccactttcccaaaactgtcactcagagtctaaagaagaccatcaggttccttcacagc

SEQ ID NO: 85 - DNA, 570 bp, Homo sapiens
tcctaatgcctaaatcagggcctaggtagtgagctgtgggcacatattaagtattggttaaactaaaaataataagcaaaatggacattatctataaaagcttttgtggaaatggctagagctagggtaaggaaacaaatttggttccccatacctgccctccaagaaaataaagctgtcaaggaaaatctgggctaagagtaggatatgagggatgatggataaggcatgagacatgagaaataagggggattaaattattattactattatacaaatgatgcctgagtagatttttaaaatgattaaatacccaatgatgtaaaaaacatttataaaataggaaagtaagactgactcaaccataatttgttgagtcaacccaaaaatctatttggttattttcaaacagaaatagcctacagatgatatctgagattgttccaaactttttctatgaatatgtatactttttttacataattaacataatactgtatattaatttgttacctgctttttcaattaacaatatatcataagcatctatgccaataaacacaattctgc

SEQ ID NO: 86 - DNA, 382 bp, Homo sapiens
gaagacttactgttggccatagttaattgtgtgaggaacacgccggcctttttagccgaaagactgcatcgagccttgaagggtattggaactgatgagtttactctgaaccgaataatggtgtccagatcagaaattgaccttttggacattcgaacagagttcaagaagcattatggctattccctatattcagcaattaaatcggatacttctggagactatgaaatcacactcttaaaaatctgtggtggagatgactgaaccaagaagataatctccaaaggtccacgatgggcttttccaacagctccaccttacttcttctcatactatttaagagaacaagcaaatataaacagcaacttgtgttcctaacagg

SEQ ID NO: 87 - DNA, 486 bp, Homo sapiens
ctggagaagcactgccattcagcctcctgctccagctgttcacatgcagaaatgctctcttcacaggcagagaagcctgtggctaaagtttccacatcccattaactcagtgcttttgtctttttcatgacatggcacatagagaaaatatttttttctagcacacaagagcaacctgaaaggctgctcctggctaggggactctgtcccgggggaccgtgtcctcccccatgtcctgcctaggccctcagaggaccaggggatcatgtctccaggtaacccgactgtagcccctgctggctgagctccagcctgtgcccactgataatagcagggacggcctttctcttagagcagctgataagtttccctacctgatggccccctctgacataaactgcacacctggggtgatggcttaaagccagaaagagctgagggagttaagagggccaaccttagggcacgtgggcattattaaaggtc

SEQ ID NO: 88 - DNA, 374 bp, Homo sapiens
  misc_feature (202)..(202): n is a, c, g, or t
tgccccgcaagggttctggcagtgactgccgtctgggccacaatgccttctcctgggtcttctctcgctatgatcaggagtttcgtttctcacacaatgggcagcacgagcccctggggctgctgcggggcccagcccagctgggtgtagtgctggacttgcaggttcaggagctgctcttctatgagccagcctccggcanagtgctctgtgcccatcatgtgtccttcccggggcccctcttcccagtctttgctgtggccgatcagaccatttctatcgtccgctgacctctggccacaggaagccaggtccaccgcccaccaccctttcaggccatgtttctactcagtgtgcttttcccaaatgatgtg

SEQ ID NO: 89 - DNA, 487 bp, Homo sapiens
aactcatacgtcctgtggtggcattgggagagttcccccatgatgagggccaagatagaatctgtaccactcagtgctaccatccccacccctacaccacttccacacaggggcctcatggcatggtcagggtcccagctgtaggtgagagcagggcactgtccagctgtccactggggaagtcaagatgtcctaaggcccaggtcagggcatctggagtctgaaggaccctagttcctagaggcatctggcagcaagaaggtgaggcatcagggaacgggaatcaggctgggactgatcagaggtgaagggacagagagaggagaggaggaagattgagctgggggcaacagccaagctcacctgggcaggtctctgccacctccttgctctgtgagctgtcagtctaggttattctctttttttgtggctatttttaattgctttggatttgttaaatgttttctgtcttctgttaagtgtgttt

SEQ ID NO: 90 - DNA, 380 bp, Homo sapiens
cctatgcgaatgacgtgtatccctgtacccatggtaaatgtaaatatactgtgtctcttttgggagagccttttagtagaggagtcttatatgagtctctacataagtagtttcacttgagttttgcagtttgaaatcttaaaggagctttaattgacatttattataccaattaagcttggaatggggcaatggatgcatttcccaaaacgtgtgaaagcactaacagcttatattgctgaatgagaatctcctgggtgtaatttagccacttagggaactgcgtgaacactcccaggccattatgatgctgttacagcttcagtgtataaatgcatgagtattctttctgttctgttttgtgctctcttgtacattta

SEQ ID NO: 91 - DNA, 518 bp, Homo sapiens
gaaagcaccctaattgacttggaaaaaaaaaacagcaaaagcaaaagtagcaacatatgtcaacatatgtcactgaaataggaaacagtcattggaatgttgcacagaggctaatagctatggactgttggatacaggatacagtggtgagaggagccccattttaggtctttcttttaggtttttggttttcattactccaagtagcccttgacccaagaacaaaggcttgttgtatgagttccactgccagatttatgggatgcctggatcattcagaaggatgcttcaactattatttgtcaggtccaaaggtcgtacttgataaccccattttctatgtatggggtagtctaatatattattttatctactttatttttcccttttcagaaagtccttagtgcaaaccaccattggaatctagtcagaaatgtctgtcagatagttagaattgtaacatctaaacctgccacggatcgaatggtacttacaggtacctctcttagggactctgt

SEQ ID NO: 92 - DNA, 313 bp, Homo sapiens
aggaaagtgggcgcagagccctcctgctcacacagctgctgagacttcagggacccatcagaacttggtgcagcacagccccgcccgtggagggtcccttttacgcaccccaaggcccacacctaagcttccatgtagccctcatccagggaagttttgcgatcctttaggaagacactgtcctcttattacagattgtgtatttccgtaggcttcttagtagcagctttgtacactgaggacactgtagccaggaacctgtgcatgccacccaccgcctggacaggcagtcatcctgcctctgatgtgaatc

SEQ ID NO: 93 - DNA, 201 bp, Homo sapiens
tgggagcatggtgagcagccctggtgctcagcagccatacctatgggacacacactacgaaaaggatgcctttagggtttgggggagattttactcctttcttcaacaactattcactggacaagttctctgctcccatgacgcgccaggcacagttctgcaagtatattgtgaatgtattgttctagtgggatacacaaa

SEQ ID NO: 94 - DNA, 51 bp, Homo sapiens
tttcactgaggccatctatccgatttcaaggaagccaagggcacatctcca

SEQ ID NO: 95 - DNA, 411 bp, Homo sapiens
ggtccgccttctggatgtgtatgaaaaggaagtggtcaagttctcagcctcacctgacccggtccttcagtggactgagaggggctgccgacaggtctcccacgtcttcaccaactttggcaagggcatccgctacgtatcttttgagcagtacgggagagacgtgagttcctgggtggggcactacggcgcccttgtgacccactccagtgtgagggtcaggatccgtctgtcctagcgactggactactgcctgacgttgtcagtcaagaccagccttgcagccaggtgcagtggctcacacctgtggaatcccagcacttcaggaggccgaggtgggaggatcacttggagctcgggagttcaagaccagcctgggcaacatagtgaaaccgtctccacaaataattt

SEQ ID NO: 96 - DNA, 515 bp, Homo sapiens
tgtctccccgctatctaaaaaagtataagaacaagcagataacagcgagaatcttggaggcccatcagaatgtagctcagatgagtctaattgaagccaagatgagatttattcaagcttggcagtcactacctgaatttggcatcactcacttcattgcaaggttccaagggggcaaaaaagaagaacttattggaattgcatacaacagactgattcggatggatgccagcactggagatgcaattaaaacatggcgtttcagcaacatgaaacagtggaatgtcaactgggaaatcaaaatggtcaccgtagagtttgcagatgaagtacgattgtccttcatttgtactgaagtagattgcaaagtggttcatgaattcattggtggctacatatttctctcaacacgtgcaaaagaccaaaacgagagtttagatgaagagatgttctacaaacttaccagtggttgggtgtgaatagaaatactgtttaatgaaactccacggccataa

SEQ ID NO: 97 - DNA, 536 bp, Homo sapiens
  misc_feature (26)..(26): n is a, c, g, or t
aaaatgctattagtccgtcgtgcttnatttgtttttgtccttgaataagcatgttatgtatatngtctcgtgtttttatttttacaccatattgtattacacttttagtattcaccagcataancactgtctgcctaaaatatgcaactctttgcattacaatatgaagtaaagttctatgaagtatgcattttgtgtaactaatgtaaaaacacaaattttataaaattgtacagttttttaaaaactactcacaactagcagatggcttaaatgtagcaatctctgcgttaattaaatgcctttaagagatataattaacgtgcagttttaatatctactaaattaagaatgacttcattatgatcatgatttgccacaatgtccttaactctaatgcctggactggccatgttctagtctgttgcgctgttacaatctgtattggtgctagtcagaaaattcctagctcacatagcccaaaagggtgcgagggagaggtggattaccagtattgttcaataatccatggttca

SEQ ID NO: 98 - DNA, 555 bp, Homo sapiens
  misc_feature (143)..(143): n is a, c, g, or t
caaagccctcactgctggagagaaggccatagcagcggagcgggccatgaaggaagcagctgagaaggaacaggagctgctaagagaaaaacagaaggagcagcagcaaatgatggaggctcaagagagaagcttccaggaanacatngcncaantnnagaagaagntggagnnggaaagggaaaaccttctcagagagcatgaaaggctgctaaaacacaagctgaaggtacaagaagaaatgcttaaggaagaatttcaaaagaaatctgagcagttaaataaagagattaatcaactgaaagaaaaaattgaaagcactaaaaatgaacagttaaggctcttaaagatccttgacatggctagcaacataatgattgtcactctacctggggcttccaagctacttggagtagggacaaaatatcttggctcacgtatttaagagcctgaatattccagnnnngnnnananannnnnannnnnnntnnattttaataacataacactgttgctcattttgtaagtataagtgttatagcagtttcattcaag

SEQ ID NO: 99 - DNA, 509 bp, Homo sapiens
gtcagagctgatcgtgcaggctgcttcctcctctaaggagcgtggtggtgtgtcgttggcagctcttaaaaaggcgctggcggccgcaggctacgacgtggagaagaacaacagccgcattaagctgggcattaagagcctagtaagcaagggaacgttggtgcagacaaagggtaccggagcctcgggttccttcaagctcaacaagaaggcgtcctccgtggaaaccaagcccggcgcctcaaaggtggctacaaaaactaaggcaacgggtgcatctaaaaagctcaaaaaggccacgggggctagcaaaaagagcgtcaagactccgaaaaaggctaaaaagcctgcggcaacaaggaaatcctccaagaatccaaaaaaacccaaaactgtaaagcccaagaaagtagctaaaagccctgctaaagctaaggctgtaaaacccaaggcggccaaggctagggtgacgaagccaaagactgccaaacccaagaaagcggcaccca

SEQ ID NO: 100 - DNA, 409 bp, Homo sapiens
  misc_feature (53)..(53): n is a, c, g, or t
tccttcttactctgcagcaacatggaggagagttttgtgtagtgagtgtgggngaagaaatacatttggctgttctcacaccccctctgactatgcaccagtgaacacatctgagtacataccagctctcctcatcttcttatttatacttaacttatttttgtgtgaaataaatggaggacgaaatcttagagcaacatcatcaaacagtctttggtccttgagaatcttctttgtgttttattttttgatttctgtagcttttcagttgcagatgttgaaattcgtaatgacaaatatgacaaattgtcatgggtgattccacttcatcttattttttctactctcactatacaatcttgcctcattttttaaaactttggaaccagaggatttcaactgcctagca

SEQ ID NO: 101 - DNA, 140 bp, Homo sapiens
atcacagctcacgtacttaggagtttagcttaagtcgtcatatataaaatacagtctctaatatcatgattgatggagagcattcagcttgccttagatgctgtaaattcaggaaaagctgaacattaaggcgtttttgt

SEQ ID NO: 102 - DNA, 523 bp, Homo sapiens
  misc_feature (29)..(29): n is a, c, g, or t
atccgacttgaatattcctggacttacanaatgccaagggggtgactggaagttgtggatatcagggtataaattatatccgtgagttgggggagggaagaccagaattcccttgaattgtgtattgatgcaatataagcataaaagatcaccttgtattctctttaccttctaaaagccattattatgatgttagaagaagaggaagaaattcaggtacagaaaacatgtttaaatagcctaaatgatggtgcttggtgagtcttggttctaaaggtaccaaacaaggaagccaaagttttcaaactgctgcatactttgacaaggaaaatctatatttgtcttccgatcaacatttatgacctaagtcaggtaatatacctggtttacttctttagcatttttatgcagacagtctgttatgcactgtggtttcagatgtgcaataatttgtacaatggtttattcccaagtatgccttaagcagaacaaatgtgtttttctatatagttccttgccttaa

SEQ ID NO: 103 - DNA, 256 bp, Homo sapiens
gagcaggacgaggagcttgaccacatattatcccctccacccatgccgtttcggaaatgcagcaacccagatgtggcttctggccctggaaaatcactgaagtataaaagacagctgagtgaggatggaagacagctaaggcgagggagcctgggaggagccctgactgggaggtaccttcttccaaacccggtggcgggacaggcctggccggcctctgcagagacgtccaacctcgtgcgcatgcgcagccagg

SEQ ID NO: 104 - DNA, 232 bp, Homo sapiens
  misc_feature (128)..(128): n is a, c, g, or t
ctgttatctgtggtgagcaggttggtgtcaaaggaaatccccaaggcttcaaccagggtctggattgtgatgtgatcgtagctgaggtatgtgcttcccaggcctccaaagcttccacatttttgttnggtatcagttatncatgttngggtgtattctcatcccagatttttctcatttagatcataaacataatagaaagggctagaattgcaatcttgtgtaacttaaa

SEQ ID NO: 105 - DNA, 228 bp, Homo sapiens
  misc_feature (55)..(55): n is a, c, g, or t
gaaaaaatggttgacctcacccaggtaatggatgatgaagtattcatggcttttncatcctatgcaacaattatnctttcaaaaatnatgcttatgagtactgcaactgcattctatagattgacaagaaaggtttttgccaatccagaagactgtgtagcatttggcaaaggagaaaatgccaagaagtatcttcgaacagatgacagagtagaacgtgtacgcaga

SEQ ID NO: 106 - DNA, 255 bp, Homo sapiens
aagaacctgctgcggctacaggacctggtggacaagctgcaactgaaggtcaaggcctacaagcgccaggccgaggaggcggaggagcaagccaacaccaacctgtccaagttccgcaaggtgcagcatgagctggatgaggcagaggagcgggcggacatcgctgagtcccaggtcaacaagcttcgagccaagagccgtgacattggtgccaagcaaaaaatgcacgatgaggagtgacactgcctcgggaac

SEQ ID NO: 107 - DNA, 120 bp, Homo sapiens
ctgcagaagctggaagcgcgggtgcgggagctggagaatgagctggaggccgagcagaagcgcaacgcagagtcggtgaagggcatgaggaagagcgagcggcgcatcaaggagctcacc

SEQ ID NO: 108 - DNA, 536 bp, Homo sapiens
gaccatggaacctctgatccaagcagcccagctcctgcaattaaagaagaaaacccaggaggacgcagaggctatctgctccctgtgtacctccctcagcacccagcagattgtcaaaattttaaacctttatactcccctgaatgaatttgaagaacgggtaacagtggcctttatacgaacaatccaggcacaactacaagagcggaatgaccctcagcaactgctattagatgccaagcacatgtttcctgttttgtttccatttaatccatcttctctaaccatggactcaatccacatcccagcgtgtctcaatctggaattcctcaatgaagtctgaagatgcatgtttccagcattagtttgattcccaatgtgagcaagaaggaagtatatacagtaaagtaaattcaaggatctgttaaatctggtaaaagtagatcaaatcagagattgacagcctgtggagggtgctgaactatacagaattagacacaactatgtcattattttttgtacctactgctcagaat

SEQ ID NO: 109 - DNA, 484 bp, Homo sapiens
cagaaactttcccagaagatggaggagagtatatgtgtaaagcagtcaacaataaaggatctgcagctagtacctgtattcttaccattgaaagtaagaattaatcactctttttatcttttattctattaatttttttttccttaaaatcacttttcttcttctcttttttagctgatgactactagctcccctcccctctccctggaactttctctttcactccaactttcttactacatccatcttttctgtggcggggccaaaaaaggaaaccaggagtgccactatgctgacttcttattccttttcataacagtcttcaaagcacagctcatctaaagaatgcctacttcttttccaaataagcatcagatttatcgcctattatgcagtaacagtcaataaaatgtacttatgggggggaattactcaattattctatcagaacctattataaagactgtatttcccatagacgttt

SEQ ID NO: 110 - DNA, 205 bp, Homo sapiens
  misc_feature (38)..(38): n is a, c, g, or t
agatttcctcaagtgtgcatagatctgagaggagtcgnggagtcgagactcgagattccatcatagcgtaggtgtgtggggttgggagccccctgatgggcttgtctgtgtttgcaccttgtcctgtgtctgaggtcctgtgactgtaccctcctttgccctgggacatctgtatctcttggctttgtaataaatgctgcatact

SEQ ID NO: 111 - DNA, 198 bp, Homo sapiens
catacgctggacgagtcggaccgaggctaggacgtggccggcgctctccagccctgcagcagaagaacttcccgtgcgcgcggatcctcgctccgttgcacgggcgccttaagttattggactatctaatatctatgtatttatttcgctggttctttgtagtcacatattttatagtcttaatatcttgtttttgca

SEQ ID NO: 112 - DNA, 530 bp, Homo sapiens
  misc_feature (371)..(371): n is a, c, g, or t
ggagttcattgcaccagttacactgaaggtctttgcaggctattatacgaagggatttgcactactgaattttgtagtaaaatactcccctgaacgacagcgttctcttcgtcctcatcatgatgcttctacatttaccataaacattgcacttaataacgtgggagaagactttcagggaggtggttgcaaatttctaaggtacaattgctctattgagtcaccacgaaaaggctggagcttcatgcatcctgggagactcacacatttgcatgaaggacttcctgttaaaaatggaacaagatacattgcagtgtcatttatagatccctaagttatttacttttcattgaattgaaatttattttggntgaatgactggcatgaacacgtctttgaagttgtggctgagaagatgagaggaatatttaaataacatcaacagaacaacttcactttgggccaaacatttgaaaaactttttataaaaaattgtttgatatttcttaatgtctgctctgagccttaaa

SEQ ID NO: 113 - DNA, 502 bp, Homo sapiens
ttatcaagtgtcaagatcagcaagtgtccttaagtcaaataggtttttttttgttggtggttgtgcttgctttccttttttagaaagttctagaaaataggaaaacgaaaaatttcattgagatgagtagtgcatttaattattttttaaaaaactttttaagtacttgaattttatatcaggaaaacaaagttgttgagccttgcttcttccgttttgccctttgtctcgctccttattcttttttggggggagggttatttgcttttttatcttcctggcataatttccattttattcttctgagtgtctatgttaacttccctctatcccgcttataaaaaaattctccaacaaaaatacttgttgacttgatgttttatcacttctctaagtaaggttgaaatatccttattgtagctactgtttttaatgtaaaggttaaacttgaaaagaaattcttaatcacggtgccaaaattcattttctaacaccatgtgtt

SEQ ID NO: 114 - DNA, 493 bp, Homo sapiens
taagcacgttgcatgacatctggaacacagaaagccctcaatacattgaagctcttaggattttcacgatgttcctgtctgctcaatgcatgctttctttattgttctgacagttgtgtggtaacaagctaatatgcttccagttgacttccagtctaccctggtgttagaaaccgtttcatctcttattgtaaatttgagtgcttgttgttttttatatttgtgatgactcttccagcagttgttgacaattgttagaggtttgacttttaaataattacttattttttctgattgtggttcagtttaactgaagaatatcctgagattgtaagaaaagcattttttaaaaggtatcacttgtgatcatttatctttctaaattctatttttaatactgttccaccaaagtgatgcagtggttaccatgacaccctaatttcatgtgtttttgtatttatgaaaatagtttcattgtcatttattggcggta

SEQ ID NO: 115 - DNA, 291 bp, Homo sapiens
gttgagtcaacgggggtgttgccaccagatgtgctggtgagtgaagccatcaaagtactgatggggaagtgccggcgcttcttggatgaactagatgcggttcagatggactgagcttggatgcttctgaggcaagctgaagctttgggttctgactgacccaccctacaggactgctgaacagagagcccagtgtgactagggatcctgagttttctgggacaattccagctttaatcaatacattttgttaaatgtgccataaaatgagactttttacgcctttataag

SEQ ID NO: 116 - DNA, 449 bp, Homo sapiens
tgtttctaacacgcttgcaacttcccttatggcattaatcttgttgagggagagagacagaatcctggactctccaaagtatttaactgaaagtagggcctgctctgacagggcccatgtcccacaaggctgcttggcctcagtgggtgcttggctgtgctggatgatatgttgatctgtattggataaggaccaatgacagcaaagcaaaaatggctttaaagcttggtgttacttttcttaagttgtttaattatagttaagcaatttcaaaaatgctccaaagaaatgtgaaaggaccttttgtcacagcacttcagaaaatacacaacagccccttctgcccccgcacagaaatgctgcagagtatataaaacttgagacatttttgtaggatgcctgacgaggtgtagccttttatcttgtttccggatgcatatttattacga

SEQ ID NO: 117 - DNA, 386 bp, Homo sapiens
gtagcatgttgtcagtggccaaggctacacagaaggctccctgctgcccggagcaggtacatccaccagagcaaagggaaccacttttattttgcatgagtttggtaactgattactctcccctcaaagaaaagacattcaggtgtttctcaacgacatcttctgtccagcaagctcggtttgaatacgtcacttaccagtgccattgcaggacccaaattcacagttcataaaagatgtgaccactacatgtaaaaatagcattctacttgatcttacagtatgtatgtatgtatgtatggagacatatgtgtgtgtgtggatgtctacatggttaatggaaagcactgtgctctgaagtggatcagtctcaagtgtctggtaac

SEQ ID NO: 118 - DNA, 328 bp, Homo sapiens
  misc_feature (263)..(263): n is a, c, g, or t
ggacccagtgtgcgatggctgcacacagcagcttccttggtagtgtacgcagcctgttggttgtatgggttgctctaagggaccttggagacaggcctttcaggtggatgttcatgtttctgaccttgcactaccccaatgtaggctccaaacaggcatgcgaggtgcctttggaaagccccagggcactgtggccagggttcacattggccaagttatcatgtccatccgcaccaagctgcagaacaaggagcatgtgattngaggccctgcgcagggccaagttcaagtttcctggccgccagaaggtatgtagtgctgcagcccc

SEQ ID NO: 119 - DNA, 190 bp, Homo sapiens
gtgagagagaaaggtgctggcataggatctgcccagaagagaaaatgacccatgcgcagttgggctctggatacggcgctgtctatagcaagttggccagtctggcctcctgttcctctgctggacctggggtaggctgcaggggtgggcagaagcccctcttaaattgtggttgccatggtaccgaggg

SEQ ID NO: 120 - DNA, 478 bp, Homo sapiens
gcaaaatgtcgcagctggaacgcaacatagagaccatcatcaacaccttccaccaatactctgtgaagctggggcacccagacaccctgaaccagggggaattcaaagagctggtgcgaaaagatctgcaaaattttctcaagaaggagaataagaatgaaaaggtcatagaacacatcatggaggacctggacacaaatgcagacaagcagctgagcttcgaggagttcatcatgctgatggcgaggctaacctgggcctcccacgagaagatgcacgagggtgacgagggccctggccaccaccataagccaggcctcggggagggcaccccctaagaccacagtggccaagatcacagtggccacggccatggccacagtcatggtggccacggccacaggccactaatcaggaggccaggccaccctgcctctacccaaccagggccccggggcctgttatgtcaaactgtctt

SEQ ID NO: 121 - DNA, 535 bp, Homo sapiens
  misc_feature (98)..(98): n is a, c, g, or t
cctgtcccatggagagggacccagcataggaaagccacatactcagcctggatgggtggagaggctgagggactcactggagggcaccaagccagccnacagccagggaagnngngagggggggncggnnncccatgcctnccagctgagcactgggaatgtcagcccagtaagtattggccagtcaggcgcctcgtggtcagagcagagccaccaggtcccactgncccgagccctgcacagccctccctcctgcctgngtggnggaggctgnaggtcattggagaggctggactgctgncaccccgggtgctcccgctctgccatagcactgancagtgncaatttacnggaatgtagcagcgnatgnnattncctggaacagttttcgttaagtatagtaaggttttagtgggggggggcaactaaacaaacacaaagtattctgtgtcaggtatagggctggacagggcagttgtgtgttggggatggtaaattactctatttaaagtaggtcttgtgttctaataatgtt

SEQ ID NO: 122 - DNA, 525 bp, Homo sapiens
tcatgcatctgttctacgtgcgggcgggagtgtctcgtgccatgctgctgcgcttcttcctggccatggagaagggcaggcatatggagtatgaatgcccctacttggtatatgtgcccgtggtcgccttccgcttggagcccaaggatgggaaaggtgtgtttgcagtggatggggaattgatggttagcgaggccgtgcagggccaggtgcacccaaactacttctggatggtcagtggttgcgtggagcccccgcccagctggaagccccagcagatgccaccgccagaagagcccttatgacccctgggccacgctgtgccttagtgtctacttgcaggacccttcctccttccctagggctgcagggcctgtccacagttcctgtgggggtggaggagactcctctggagaagggtgagaaggtggaggttatgctttggggggacaggccagaatgaagtcctgggtcaggagcccagctggctgggcccagctgcctatgtaaggccttctagtttgt

SEQ ID NO: 123 - DNA, 530 bp, Homo sapiens
gggtccacaatatttccagactccacacaacccttcaggtcctggcctgggcaaccagctgatgcgactcagccttatggaagaggacgaggaagaagagttagaaatccaggatgagtcaaatgaagaacggcaggatacagacatgcaagcaagtagctactcttccaccagtgagcgcgggctggacctcttagattctacagagctggatattgaggatttctgacaggactctgggcccctccccagctccactccctacctcaagaatgtgaccatttggaaaaggcaaagagaaaaggagcaaaatgaagcattcccccaggcttcagccctgggctctgaggggaaagagttgggcattgtttttctaacctaacctttccctctggggtagagaagccgagagaccctgtcctccctaatgcactgtggcccagtccccttgcctttttcctgttctgtttggagtggagaagggcagcacctctgtgtttaatggaaatagcccatagtctcctggattt

SEQ ID NO: 124 - DNA, 539 bp, Homo sapiens
  misc_feature (31)..(31): n is a, c, g, or t
aacatcgaggatgaaggcagctccttctatngggtctccagccagtatgaganccccgagaannnnnnnnnnnnnnnnnnnnnnnnnnnnnnnnnnnnnnnnnnnnnnnnnntggagaaagttgagacagagtatgctcgctatgagaatggacactactcttaccgcatccaccggtccccgctctgtgagtacatgatcaacttcatccacaagctcaagcacctccctgagaagtacatgatgaacagcgtgctggagaacttcaccatcctgcaggtggtcaccaacagagacacacaggagaccttgctgtgcattgcctatgtctttgaggtgtcagccagtgagcacggggctcagcaccacatctacaggctggtgaaagaatgagagactcggggagcagggaggggggaagagacgtgtgtgcaggaaacggggacgtggggaggggacctgcaggggcagccccctgaagtgccaagagagctgagaggagcagttgtgactctacccaggaacaaactgtgcctgaa

SEQ ID NO: 125 - DNA, 375 bp, Homo sapiens
ttacttagtccttaggccaaccaatttaactgcagtgtcatgtttcacaggccttcctacatttagaaatcgtcacacagctgtgataagagtagattattttactatgaaataattctgaatagatgaaagcataaaatgtgagaaactgaatgtattattcaggaagaatactgagtgccttcatttaactaaagttgaatgtaaaagtcaatttgcacttctttataatcctctggtttagaattataaattgttaaaaccttgataattgtcatttaattatatttcaggtgtcctgaacaggtcactagactctacattgggcagcctttaaatatgattctttgtaatgctaaatagcctttttttctc

SEQ ID NO: 126 - DNA, 502 bp, Homo sapiens
agcccatgatgccgtggcacaggaaggccaatgtcgggtagatgacaaggtgaatttccattttattctgtttaacaacgtggatggccacctctatgaacttgatggacgaatgccttttccggtgaaccatggcgccagttcagaggacaccctgctgaaggacgctgccaaggtgtgcagagaattcaccgagcgtgagcaaggagaagtccgcttctctgccgtggctctctgcaaggcagcctaatgctctgtgggagggactttgctgatttcccctcttcccttcaacatgaaaatatataccccccatgcagtctaaaatgcttcagtacttgtgaaacacagctgttcttctgttctgcagacacgccttcccctcagccacacccaggcacttaagcacaagcagagtgcacagctgtccactgggccattgtggtgtgagcttcagatggtgaagcattctccccagtgtatgtcttgtatccgatatcta

SEQ ID NO: 127 - DNA, 244 bp, Homo sapiens
cagtataatgactcacccggaaggtttcttaattcgttcttccatttatttttaaaaattttgtttgaacgcctactaagttctgggtgcagggtataacacagcaagcaccatggaaaggtccctgctcctagtgctcacactccaataagaagaagtggctgggccgggcacagcggctcacgctgtaaccccagcatttcgggaggcctgggcaggcagatcacctggggtaaggaatttg

SEQ ID NO: 128 - DNA, 116 bp, Homo sapiens
gtcttgaggatacacgggacccagaatccacggggtctggagcataaggtttatctcaagtctcaattgaactgcctcctcttgtcaggcagggacaactgggaggatgagcccca

SEQ ID NO: 129 - DNA, 547 bp, Homo sapiens
ggaccagctcccctagaggagataaagcagcctatgacattctgaggagatgttctcagtgtggcatcctgcttcccctgccgatcctaaatcaacatcaggagaaatgccggtggttagcttcatcaaaaaggaaaacaagtgagaaatttcagctagatttggaaaaggaaaggtactacaaattcaaaagatttcacttttaacactggcattcctgcctacttgctgtggtggtcttgtgaaaggtgatgggttttattcgttgggctttaaaagaaaaggtttggcagaactaaaaacaaaactcacgtatcatctcaatagatacagaaaaggcttttgataaaattcaacttgacttcatgttaaaaaccctcaacaaaccaggcgtcgaaggaacatacctcaaaataataagagccatctatgacaaaaccacagccaacatcatactgaatgagcaaaagctggagcattactcttgagaagtagaacaaggcacttcagtcctattcaacatagtactggaagtctcgccacagca

SEQ ID NO: 130 - DNA, 546 bp, Homo sapiens
ggagaacctactgcaggaagtcagccctgactcaccatcagagaacacacacaggacagagaccctatgagtgtaatgaatgtgggaaaaccttctgtcagaagttctcctttgttgaacatcagcgaactcacactggggagaaaccatatgaatgtaatgaatgtgggaaatccttctgccataagtcagccttcagagtccatagaagaattcacacaggagagaaaccatatgaatgtaatcaatgtgggaaaacctaccgtcgcctgtggactctcactgaacatcagaaaatacacacaggagagaaaccttatgaatgtaacaaatgtgagaaaacatttcgccacaaatcaaactttcttttacatcagaaatcccacaaggaataagttccaacaaagtaataaattccaacaaagcaacaaatcaaataacttacacaatgctaaactgtgagtatgtatagaggagtaaaatcttataaatgtaatgaatatggaaaatttttctgccataagtcaaccctcaatgtaacatcag

SEQ ID NO: 131 - DNA, 511 bp, Homo sapiens
  misc_feature (205)..(205): n is a, c, g, or t
gtccagggagaggcgggatcacggcccaggctgctgacacccgcgcgtggggaggactcgggccacgaaaggaaagaaatgtataccgtatctatctacccgacagcagcgaccgagacccggtgggacactccccttctccccactttcacctccccacccaaactttataaaagttgaaaaaatatcatttgactttttatangaaaaaaaaanggaaaaaataattgangaaagtgttcatctgaggactgcatcgngtggacactggtatttatttatgttagctccaagcggaccggtggttcaaaagtgcattatttagtttgagctctgtaggtaaaaaggaggtgggaaaaattttaaaacttgagggtnaaaatgtggaanacaaaccctcccatcccttgtagattataaataaaagcaaaaccgccacagaactagaggtcttctctttaatgttactttaaaattgctatgattgtattgtacgttatttaatgtctga

SEQ ID NO: 132 - DNA, 259 bp, Homo sapiens
gaagtggtggacctttcagcagccacatagatagaagagtgaaccgacacagtgtgttatcatagaggagacaaaaatggtagtgaagcttgtggttatgtatcttatctttctacatttacatgttttctgtaaggagtgtggttatagagagactgttggaaccatgagaaggatgtttctgtgttcttgattatattgttctgcaaaactgctaagccatgaacagttttcttagctacttagaatggttatacat

SEQ ID NO: 133 - DNA, 348 bp, Homo sapiens
aagacctgtgaagtatcctagtcagggaaagaacctgttttctggaagaatgtaagttgctatatgtggaggggcatgtaggacaggggttctaattactgtctgtgagagttactactttgtaacttatggtttctgcttcagtattgtgttggttctgtattataaattgcaccttaatagaccaaaaatatctatacagcactgatttccttgaatattgtgtgttaattgatatatcttctagaggcaaaaggtttaaatgtgttaaaatggttatattgcctcctttttaaacaaattaaagagttgtaaatactagtgtagatctcttttgtttaccacagc

SEQ ID NO: 134 - DNA, 355 bp, Homo sapiens
tcttgcatactcagcttgctttatcatttttgctgtccttttaacatttgcgaggagtaggggcctggtcctgaatgacttggaggctttcattatttatcctgtctgtattgaccggtttttgttttttcagaaggcagtgatgatgaaaacttaggaagaaggtattttgcaataagcttggctgagtgtccatgggaagaatactttccctaaagagagagaagcactcacagaggctgcctttctcctgagctcggggagaaggcagcacatcacaacctgtcacattgaaataggcgctcattctgctatttcaccttccgtcctgagcagagcctgaattacgtttttg

SEQ ID NO: 135 - DNA, 80 bp, Homo sapiens
atcttcgaaatgcttgaaggagtgcaaggacctactgcagtcaggaagcgattttttgaatccatcatcaaggaagcagc

SEQ ID NO: 136 - DNA, 528 bp, Homo sapiens
  misc_feature (29)..(29): n is a, c, g, or t
ttctctggagatgacctagaatgcagagnaanagcctcctctcccaaaagccaacnagaaattaatgctgatataaaangtnaantagtgaaggaantccgatgcnttggacnaaaatatgaaaaaatcttcgaaatgcttgaaggagtgcaaggacctactgcagtcaggaancgattttttgaatccatcatcaaggaagcagcaagatgtatgagacgagactttgttaagcaccttaagaagaaactgaaacgtatgatttgagaatacttgtccctggaggattatcacaccccaaatgcataatctcgttaatgattgaggagagaaaaggatcagattgctgttttctacaatggagcaggatattgctgaagtctcctggcatatgttaccgaatcaantngccttccagaggctaagaaatttctgttagtaaaagatgttctttttcccaaannannnnanntgaaannanaanntgtgttttggttattttgtattcccacctgtgctggtagatat

SEQ ID NO: 137 - DNA, 560 bp, Homo sapiens
cccaagcccctagagaagtcagtctccccgcaaaattcagattcttcaggttttgcagatgtgaatggttggcaccttcgtttccgctggtccaaggatgctccctcagaactcctgaggaagttcagaaactatgaaatatgaaatatctctgcttcaaaaaatgaggaagagcaagactgtcccctatgctgccaacatgcagtctttgtttatgtcttaaaaatgtcatgtttatgtcatgtctgtgaattgctgagtactaattgattcctccatccttgaatcagttctcataatgctttttaaataagaaaaattcagaagatgaatttcttccaatatttgaataaattaaagctcttagatacagagtagattgtattatatgctttttcctattaatactacttatagaaatccattaaaaagcaatctctgtacagtgtatttaaatatttcattgacatactgtgatctctattagtgatggatgtacaaaaaatgttttcttacccttgacttacaatgaaatgtgaaattacttgtctgaaccccgt

SEQ ID NO: 138 - DNA, 255 bp, Homo sapiens
gaagattactacacattactgggatgtgatgaactatcttcggttgaacaaatcctggcagaatttaaagtcagagctctggaatgtcacccagacaagcatcctgaaaaccccaaagctgtggagacttttcagaaactgcagaaggcaaaggagattctgaccaatgaagagagtcgagcccgctatgaccactggcgaaggagccagatgtcgatgccattccagcagtgggaagctttgaatgactcagtg

SEQ ID NO: 139 - DNA, 300 bp, Homo sapiens
agtgaagacggtgggtttctcgctgggtgcgacgtgaatttgtgaagctcaggatgcccatggattagactcatgtagtagcttaaagagtcattaggcgataggagggagaaaaccaagaagttagcagagtctggatataattcagtgtccgtaaatcccatgaagagaagctcatcagaataaaggcaatgaatttgtgccagaagagcctgtgactacatttctttcaatctctttggctgtttgtttcagtatacacactccctgttattgcttactattttatgagctctaata

SEQ ID NO: 140 - DNA, 415 bp, Homo sapiens
atcacacggcagcacagcacgggacagctctaccgcttgcaagatgtttccagccctctgaagcttcatcgaacagagacttttccagcctacagatctgagcactgacagtaactgccaagaattgttaacacacttgtgatgtgtcagccacagattcacccaggaggtcacagaatgacagcaagggaaatgacgaccaagagaagaagcttaaagtcctggctaattgtgtggtcattggaaaactctgcaatacaataattttctttattttctttttcttttttaaattcttagtgtaattgaaacgtgctctatagatattgactctgtgttccctcttttacagctggacagaaagaagtcaatgtcacgaaatgattttctattgtagatactttgtcccttgcac

SEQ ID NO: 141 - DNA, 542 bp, Homo sapiens
  misc_feature (499)..(499): n is a, c, g, or t
gtcctggctaattgtgtggtcattggaaaactctgcaatacaataattttctttattttctttttcttttttaaattcttagtgtaattgaaacgtgctctatagatattgactctgtgttccctcttttacagctggacagaaagaagtcaatgtcacgaaatgattttctattgtagatactttgtcccttgcacttctctgaatctgtccttttgtggattcttgtgattttccttccaagtgtttcagttgtatgacagtcagtattgacaataaaatggcttttaattatttgttatttgtttacaccctattcctcagttattattactgtggttctgattaactactggaaattatatttgattatatcaccaattagttaaatcagtgcttcgactcactcttatctgttctgttcaaaactatttgttcaaagaacccgttagtgttgtttacagggttacagtttctctcacatgctttcctcaccccnttaccccnctttttgaaagcctttattttgttcggagtctctt

SEQ ID NO: 142 - DNA, 528 bp, Homo sapiens
  misc_feature (73)..(73): n is a, c, g, or t
gcctggctagctggaagacgcggtatggcagggacaggagggcggcaggaagggctttgtggccctgctgggnccaaaggcagttttctgtgctccctttagccccagcctggctggccaggtctcctgtacttcaggggaccatggggccgcccaagccagtcaccttccccttccaagctctccccccatgactgttgatttctggcgcgtggaggcagagttgtgaatccacaaatgctgtagcccatatctcgtaatgtgtctgtgtccacttagagcctcacgacctggctgtgtgtgtgcatgtgtgtgtagtatagtttgtagttgacaatatgtgtatataatagctggtgtatttatatgtgagtgcagaattagtgcccgtggcggttttctgtattttaaacccattgaattggttagttttattttagggaagtgggaaaggcggagtggggggaagtataggaagaaggattcttagcaaaatcattctctaggactattagtcgcgatctccca

SEQ ID NO: 143 - DNA, 570 bp, Homo sapiens
  misc_feature (41)..(41): n is a, c, g, or t
cttgctatagatttgcagcttaataacattgttgtacaaanaaacactgtcaaggatttcaaattttnggtattntattgattctngtnttgggcctaacctcagcctatatctgtctatctaacctatctatctatctatctatctatctatctatctatctatctacctacctacctatctattatctatctacctagatagacagagatatctatagatagctatggtaaattttgaaacacgttccaaatacattataataaattgtgtgttttgctatttttgggtgcagtttttatatcaaagtatttaatcctactgctttaaatttttctattattattgatattttgtatcctttttgctatcagttattgaaaaaggtatattaaagtctttatgattgaanannnngnnannnnnnnttttagttctataaaaattgtaaatgtgtgttaactgtgtgtgagtgaatatacatacacaagtttgacaacttcctcaatagttgattcttttatatgggatgatgggatacatcttcctttcaagtaataatttctgcct

SEQ ID NO: 144 - DNA, 478 bp, Homo sapiens
cgctggaagagctcgtggcgtgcgacaacgcggcgcagaagatgcagacggtgaccgccgcggtggaggagcttttggtggccgctcagcgccaggatcgcctcacagtgggggtgtacgagtcggccaagttgatgaatgtggacccagacagcgtggtcctctgcctcttggccattgacgaggaggaggaggatgacatcgccctgcaaatccacttcacgctcatccagtccttctgctgtgacaacgacatcaacatcgtgcgggtgtcgggcatgcagcgcctggcgcagctcctgggagagccggccgagacccagggcaccaccgaggcccgagacctgcattgtctcctggtcacgaaccctcacacggacgcctggaagagccacggcttggtggaggtggccagctactgcgaagaaagccggggcaacaaccagtgggtcccctacatctctcttcaggaacgctg

SEQ ID NO: 145 - DNA, 260 bp, Homo sapiens
tgagtagggcaaattttaaatgggtattatttttcatcttcaaacaggcagacctgttatcctaaactaggtgagtcagcttttggtacatgtgatgattttcagtgtaaccaatgatgtaatgattctgccaaatgaaatataatgatatcactgtaaaaccgttccattttgattctgaggttactctactaacaagcatcacacatttgtattttgccctgattaatatgttggcttcgctttcagggtttttaatg

SEQ ID NO: 146 - DNA, 535 bp, Homo sapiens
gaaaaacatgagcactctgcagacaaaatgaccttaaatcacattgattaagatattttaaaagttagcagtgaaccaaaagtagtttcagattagcagaaataaagagctttaagttttaaaagttgagattgaatatttaatgaaagtttattaattctttttccaggaatagcagtaaggtcagtttttttccctaaaataaaaagttttaataaacagaaaattatagcaacagtacttaggagaatagttgaagattgtattaattttaattataatagttatttgtcataggctattgatgattagaatttcattagttttgtccactataattttaaaaatagttgtgtcaaatacaaattctggtaaactgttaggttttaacaccagacagtattcaaaggaaattaatgtttgcaaacataaatccatagtcttcatttctttatattgtcacctttgtaaaagtgtttaaaatttgtattgtttgttttgtatatctttgggcatcttgtgtctagctataata

SEQ ID NO: 147 - DNA, 177 bp, Homo sapiens
gccgggcgggggaatatacaaagtgaagccacattgccaaacttgcagcagcgattgcagcagttgctgccgctgcgccgcgcctgaagccgcgccgcgcgggccgagggctcctgcagctgctcgcgcgcagtcggaggcggagaaggacgaagactgagactgacacttctgctc

SEQ ID NO: 148 - DNA, 547 bp, Homo sapiens
  misc_feature (145)..(155): n is a, c, g, or t
agatgtaaagggtccattcatgaggaataacatcactttgaccctttgtgttagctcctactgctaatctttccataagcgtgaacttactttcaatttgaagaagatgaaaaagtcttttgcaaaaccccaatactgttttctnnnnnnnnnnnctgaanggagaaacgtagtcttacctcgtttttttccactacaagccaagctacttgtaatccttccaaacctttaaaggggacctcccttgttagcatctcccagagaacctggaacaaaaagaaaatggaatttttattttntgtactttatatttttntggntacatattactgagaccttatctanacatgtaanttgtatctanattaccangacacaaccaaccccaactgtagaaactnnngcaaaacaattaagcacacannttcttctatatattaaatactttttacgttcttttttataaagaaaaaacctggtttagaaattaggacatctagagccagacttaaaattttatgaaaccaatgggcctgactgatttaat

SEQ ID NO: 149 - DNA, 353 bp, Homo sapiens
ggacttgaatagaccttttacagagaagatacataaaaggccaacaaatgcatgaaaggatgttcaacatcactaataactagaagaaatgcaaatcaaaatgacagtgagatattgtcttacactcattaggatggatactatattttttttaaaaagacaagataacaagtgttagtaaggacatacagaaattggaacccttgttcattgttggtaggaatggaagatggtacatctgccttggaaaactatatggtggttctccccaaaattaaaatagaattaccatctgatccagcaattccacttctgggtacacacccaaaagaactgaaaacatggctaacatg

SEQ ID NO: 150 - DNA, 567 bp, Homo sapiens
  misc_feature (520)..(520): n is a, c, g, or t
tctgctctgttcagtttgctgttgtgtttttctcccccatgttggggtggtggggtacagggaaataaaatgctttctcccaggcccctaatccttccccatgcctccatcagcctcaaagctgctgacagtcatgaactgcaccttccagccctgcccataagctactcaaagcaaattcaaattctcttctggccaggaggaagggcagatgctccctccttcctcaagcctccctggctcattgatccattttgagggcatttgggggtcaaagttgagaccagattgcttcagtttgtataaaattagcatttcttatcacaccaaggccacacctgttctctggcctcacaaaccagtgaggatgtaaaggtttgttgaggtggaggaacagaagtgaaatgagcaatctgctccatttagaagtcagtcgcttcggctgttcattccactaatatttatctagtacctattctgtgccaagcattgtctctacctcagtttgccacaaatatgnaaaaaaaaaaattcttggaactgtgaggcttcaatgtgttgtggacc

SEQ ID NO: 151 - DNA, 521 bp, Homo sapiens
agatgaagtgtgccagctggttcccccaagccagctcatgctgctgaccactgactcagctctgaccttcacatttgctctgaagcaagtgcgttcagctgctggggcagtgatatcacatagtacatatattatttccttagtttatttccaaactggtattttaaatagacacttcgaactttgggctactctgtttaaatctgccactttctggactggaccttagtactgtaaattctttttaaagaataataatgttaccaactgctgagatttttatgtattttgtgactttgtaacaactgctattgtaataagtgtcatcttgtgggcattatacaaaggcatattataaaataataatgatatttttgtatagaagagtcaactgttcagatgtaagatgttgaaaaatgttaaaatctaaagagtaatttatcctagtggtaatggttatatgtatttgtacagtttaaattaatgtctcaaagctgtgcagtcttttgttactgggacac

SEQ ID NO: 152 - DNA, 524 bp, Homo sapiens
  misc_feature (99)..(99): n is a, c, g, or t
gggtaaataacatctgacagctaatgagatattttttccatacaagataaaaagatttaatcaaaaaatttcatatttgaaatgaagtcccaaatctangttcaagttcaatagcttagccacataatacggttgtgcgagcagagaatctacctttccacttctaagcctgtttcttcctccatnnnatggggataatactttacaaggttgttgtgaggcttagatgagatagagaattattccataagataatcaagtgctacattaatgttatagttagattaatccaagaactagtcaccctactttattagagaagagaaaagctaatgatttgatttgcagaatatttaaggtttggatttctatgcagtttttctaaataaccatcacttacaaatatgtaaccaaacgtaattgttagtatatttaatgtaaacttgttttaacaactcttctcaacattttgtccaggttattcactgtaaccaaataaatctcatgagtctttagttgattta

SEQ ID NO: 153 - DNA, 458 bp, Homo sapiens
atgaccaggttaaatccctctacatgtggtttctgtttgaaaaaaagaaaactgacatttgaacaggacttttaatttgtttaaaactctggtaattacttgtaacagtagaaaatagaagtcattcttattttagaaaaagtgacagaagcagtccagtaagattatatgtttctgtttctggtaaataccatatatgatcctcgaaatgataatatctccagaatattgttttcacccaaatttgagtagatattttaaacacctaacaaagtaaagggctaaaagccattcagatagcagtaaaacattctgtatgatgtgcaataaaacatccaagatcttttttgaaagttttatttataatatacatttttgtatgagaaaggtgattggtacagggtgcctattttagtcatggatcaaaatttgtgtaacttgcagggctttctttcttt

SEQ ID NO: 154 - DNA, 454 bp, Homo sapiens
  misc_feature (214)..(214): n is a, c, g, or t
gtacatctcggattttcctcttaacaaacaataatctccttcttgctgaccaaaagtctggacaaatcaagtcagaggttccactggtggatgtgaccaaggtatcaatgagctcacaaaatgatggcttcttcgccgtccacctcaaagagggctcagaagcagctagtaaaggagactttctcttcagcagtgatcacctgattgaaatggncnccaancnnctatcgcacaactctcagccaaaccaaacagaagctcaatattgagatttccgatgagttcctggtacagttcagacaggacaaagtatgtgtgaagtttattcagggaaaccagaaaaatgggagtgtcccaacatgtaaacgaaaaaacaaccgtctccttgaagttgctgtcccttaactggcgcctcctctctactttcatggacttgttcctttgtaatagtgca

SEQ ID NO: 155 - DNA, 249 bp, Homo sapiens
gtgtgaaggtgggctcacggctgactctctgtgcccagggctgtgctgccatcctggatacaggcacacctgtcatcgtaggacccactgaggagatccgggccctgcatgcagccattgggggaatccccttgctggctggggagtacatcatccggtgctcagaaatcccaaagctccccgcagtctcactcctcattgggggggtctggtttaatctcacggcccaggattacgtcatccagtttg

SEQ ID NO: 156 - DNA, 231 bp, Homo sapiens
  misc_feature (132)..(132): n is a, c, g, or t
tccagtacctgtgtggatcctcggcgacgttttcttgggggcgtatgtgaccgtcttcgaccgcggggacatgaagagcggcgcacgagtgggactggcgcgcgctcgccctcgcggagcggacctgggaangcgcgagaccgcgcaggcgcagtaccgcgggtgccgcccaggtgatgcgcatgcgcaccgggtagcagagctagcgctactcagtaaaaatccaatatt

SEQ ID NO: 157 - DNA, 175 bp, Homo sapiens
  misc_feature (89)..(89): n is a, c, g, or t
gatgtatccagttaaagcaggcaatagatgaaaataaaaatgctcttcaaaaattaagcaaagctgatgaatctgcacctgttgcaaanctataatcagagaaaagaagaggagcatactcttttggacaagcttacccaacaactgcagggccttgctgtgacaataagcagag

SEQ ID NO: 158 - DNA, 546 bp, Homo sapiens
agtggcatgaaggactctactgtggtgtggccccctctgcaaagtgcatttggagaccaggttccatgccaacaaactatgagctgtactatggcttcacaaggtttgctattgagctcaatgagttagatccagtactaaaagatctccttccaccaacagacgcccggttccggccagatcaaagatttttggaagaaggaaatttagaagctgcagcatcagagaagcaaagagtagaggaactccagagatctcggagacgatatatggaagaaaacaatcttgaacatataccaaaattttttaaaaaagttattgatgccaatcaaagagaagcctgggtttctaacgacacctactgggagcttcgaaaggaccctgggtttagcaaagtagacagccctgttctttggtagactgggaatgtagagctagccaacatatcacattctgaatgaataaataactatgcacaattatgtttcttatagctatgtgtggtttctgggtcaactgaaaacctaccatttgcttttctattca

SEQ ID NO: 159 - DNA, 378 bp, Homo sapiens
  misc_feature (27)..(27): n is a, c, g, or t
tgactttcaatacttcccctgaaggangnnnnnnnnnnagaaatgtctgttttgaggaaagggctttgaattccccagatactgaacaatttgtgtttgtgactgatggagaatttcaggaatgaatgagaaagcctttgcgaaactatgcaacagtttacatcagtcatgtgaagtatttgtctaaaacagagcaaactgaagaccaaattattctcctgttgaggtccgtggatggcagatttaaagggaagaaccacaaaggcttgcaaagataggagaggctccatctctaatgcatgtagaagctccttacgggtgcccatcaagagcatagcttggaagccaccatgctgtgcggaactgcgtcagggcaaa

SEQ ID NO: 160 - DNA, 555 bp, Homo sapiens
  misc_feature (372)..(387): n is a, c, g, or t
gagttccacagacagctcggggcccctggaggaggcagaggaggccccccagctgatgcggaccaagagcgacgccagttgcatgagccagaggaggcccaagtgccgcgcccccggtgaggcccagcgcatccggcgacaccggttctctatcaacggccacttctacaatcataagacctccgtgtttactccagcctatggatccgtgaccaatgtgagggtcaacagcaccatgacaaccctgcaggtgctcaccctgctgctgaacaaatttagggtggaagatggccccagtgagttcgcactctacatcgttcacgagtctggggagcggacaaaattaaaagactgcgagtacccgctgatttnnnnnnnnnnnnnnnngccatgtgagaagatcgccaggatcttcctgatggaagctgacttgggcgtggaagtcccccatgaagtcgctcagtacattaagttngaaatnnnnnnnnnnnnnnnntttntnnaaaaattnaaaagaagaggaagaaagagaaataatcaaacttgaccatgaag

SEQ ID NO: 161 - DNA, 534 bp, Homo sapiens
gaaagacttcaacaagagccaggccatcgagtcagtggaaaggaaaaagcaagagaaaaagcaagaacaagaggacaacttggccgagatcaccaacctcctgcgtggggacctgctctccgagaacccgcagcaggcagccagctccttcgggccccaccgcgtggtccctgaccgctggaagggcatgacccaggagcagctggagcagatccgcctagtccagaagcagcaaatccaggagaagctgaggctccaggaagaaaagcgccagcgagacctggactgggaccggcggaggattcagggggctcgcgccaccctgctgtttgagcggcagcagtggcggcggcagcgcgacctgcgcagagctctggacagcagcaacctcagcctggccaaggagcagcatttgcagaaaaaatatatgaatgaagtctatacaaatcaacccacgggagactatttcacacaatttaatacaggaagtcgataatgaggaacacacccttgttcccgtcattcacgtataaa

SEQ ID NO: 162 - DNA, 448 bp, Homo sapiens
  misc_feature (294)..(294): n is a, c, g, or t
gcaaacactctatatcaagacccccctccccgagagctggttgttaaacatttgtcagcacaccactgctgctgggagacagagagaagaaactgatacacgtgagcactgaaacatcttagttccagggaggtagcatttgtgttgtatttctcagaagcaagtgtcctagtgtaatgtgcacaagtggcatgaagtttaacacctaaaagactgcttctctgcttgttctgtctaatttctgtgatttcaacacttgagaaagatgtttactttgctactaaacttattggnagtgatttgtaaagttcactgtcacctgtgggcagtagttcagctaaattcccnccaggaaagtggactgtagcttttcctgcctaactggcaaaatgaaaatatgctgaaaagaatacatcttaactaggcaccagaatagctcatttccaag

SEQ ID NO: 163 - DNA, 337 bp, Homo sapiens
  misc_feature (38)..(38): n is a, c, g, or t
aaccttaggtcctactgtactcttggtgaatcacagcnctttagagctncctcanggncctaagcatcttttgttagaaaattctggctgaaattcctcatgagatgggtncagggaaccttggagtggcagcacttgtgtcacatnctaaaggcactcctctgactaaagagaagtggcagctgagcacactgcctgcccagggattttcttctgctctaaaggacattattaagacaactggtctgtactgtattgcctgatagtatcatatcaatgtcaatttcctcattttggtgattgttctaataagaatacgtaagaatgtgtccttgtt

SEQ ID NO: 164 - DNA, 408 bp, Homo sapiens
ggttccaggctttgcatctggagcctttaccggttgactgttgccttccacacaaacagcctctgaaaagcactttctccatacataattctggagaagatgagggatcttgccctccaggagccttccttcctcccccaatgaggaaatcagtcactgcactggtgcaaaggcaagcagattggaatttctgctcttcaccgattttctcagggaaagaccccttccccttgccagcagaggaacctgtagttttttccatttctttcttcagaaccaaagtatgtatcactcctcatgctcacagggattgacaggagagaattcaccaggatcttagctcaaaagacacagcctcagaatggccagatggattgcacgaaacctgacttggattcaccatcttcc

SEQ ID NO: 165 - DNA, 506 bp, Homo sapiens
  misc_feature (147)..(147): n is a, c, g, or t
cagtgtggctgttggaactcatgtcttccaacaagtcaaagcttccattccaagtgcagaaaagacctcctcaactgatgcaggtccagctgaggaatttcctgaacctgaggaaaatgctgcaagagaagaaaggaaattaaaagncgacacggagaaaagggaacagaaggatgaagaaatccctgaaaatgcaaacaatgaaaagatgtccatggaaacactactcaaagtgttcaaagggggaagtgaagcacaggactccaatagatttgccagccacctaaagatagagaacatttatgcagagggcttcataaaaacatttcaagacctaggtgccaagaacctggagccaattgaagtcgctgttctcttgaagcatccttttattcaagacctgatttcaaattattcagactataagtttcctgatattaaaataattctccaaaggagtgaacatgtacaaggaagtgatggagagagatcaccttcaagacata

SEQ ID NO: 166 - DNA, 315 bp, Homo sapiens
  misc_feature (157)..(157): n is a, c, g, or t
gcacgagcgggctcggtggttgggtccgcggcggctcggggtccgcccgcggctgccggtgcgagcggggcccggctcccctcctcccccgcccgccgccgccgctgtgattgggtggaagatggcgctggccggatggaaatcctaatgacagtcnccaaattcgcctccatctgtaccatgngngccaatgcttcggcattagagaangagattggtccagaacagtttccngtnaatgagcactattttggattagtcanttttgggaatacctgctactgcaattcagttcttcaagcactttatttttgt

SEQ ID NO: 167 - DNA, 468 bp, Homo sapiens
  misc_feature (129)..(129): n is a, c, g, or t
gaacaaacgtccagcaagatactttaatcctctattttgctcttccttgtccattgctgctgttaatgtattgctgacctctttcacagttggctctaaagaatcaaaagaaaaaaactttttatttcntttgctattaaaactactgttcattttgggggctgggggaagtgagcctgtttggatgatggatgccattccttttgcccagttaaatgttcaccaatcattttaactaaatactcagacttagaagtcagatgcttcatgtcacagcatttagtttgttcaacagttgtttcttcagcttcctttgtccagtggaaaaacatgatttactggtctgacaagccaaaaatgttatatctgatattaaatacttaatgctgatttgaagagatagctgaaaccaaggctgaagactgttttactttcagtattttcttttcctcctagtgctatcattag

SEQ ID NO: 168 - DNA, 447 bp, Homo sapiens
acacagggaagtgactttgctacaaataatgttgctgtgttaagtattcatattaaatacatgccttctatatggaacatggcagaaagactgaaaaataacagtaattaattgtgtaattcagaattcataccaatcagtgttgaaactcaaacattgcaaaagtgggtggcaatattcagtgcttaacacttttctagcgttggtacatctgagaaatgagtgctcaggtggattttatcctcgcaagcatgttgttataagaattgtgggtgtgcctatcataacaattgttttctgtatcttgaaaaagtattctccacattttaaatgttttatattagagaattctttaatgcacacttgtcaaatatatatatatagtaccaatgttacctttttattttttgttttagatgtaagagcatgctcatatgttaggtactt

SEQ ID NO: 169 - DNA, 473 bp, Homo sapiens
  misc_feature (90)..(90): n is a, c, g, or t
gtgcctataatttgccagtagccacagattaagattatatcttatatatcagcagattagctttagcttagggggagggtgggaaagttnggggggggggttgtgaagatttagggggaccttgatagagaactttataaacttctttctctttaataaagacttgtcttacaccgtgctgccattaaaggcagctgttctagagtttcagtcacctaagtacacccacaaaacaatatgaatatggagatcttcctttacccctcaactttaatttgcccagttatacctcagtgttgtagcagtactgtgatacctggcacagtgctttgatcttacgatgccctctgtactgacctgaaggagacctaagagtcctttccctttttgagtttgaatcatagccttgatgtggtctcttgttttatgtccttgttcctaatgtaaaagtgcttaactgcttcttggttgta

SEQ ID NO: 170 - DNA, 438 bp, Homo sapiens
tacgaggttcctagagctctgctgacccttggccgaaactctaaaatgtacctattaaagataaatgcttctaccaaagtaaaactctgtgagttgtttcagggcagaatgtaccagccagtcagcgttgtttaacaaaataatcagatttttgcctagcactcggttttggtggagctgacgattttgagggctgaggctggttaggtagctggaatgtgcctatgtgaccagctcacttgcagacaccctgccggaagcagagcttaatcttcctaggactgaggtcttagcacatgtactggtggagtttccagaccaccagtatgaataaaagcttgttctgtgtgacccagcaagtggaaggacaaagaactgtgagcctcagatctttggacctttccaatgcgtctctttctcctgttattgctgcaatgt

SEQ ID NO: 171 - DNA, 468 bp, Homo sapiens
  misc_feature (38)..(39): n is a, c, g, or t
ggagctcgattcacgtacagcacttctgtggggtttgnnaaaaaaaaaaggaaaacaacagaagaacacatcatatgcaactaatgatctcattatttaagagtcccctgttacttctttagtcatttcctttgactctgctacagataggattataggatgatgctncaaaggggaccttgaacctattcaccattatttgtctctttaagctggcaaacccatcattaaatagcacataaaatagcaatcatatgggataagtagtacagcttcagtaatcaatgggcagtggcactagaaaaatcttgagcacagtgaatgacctatcctgcaaacatctaatggatctctaaagggtaacaaaccctataaattctggcttactgcacatatttagtgtgttttaagataggatcctaactgtatatatttataactaatgtaaggaccctactttattgccaa

SEQ ID NO: 172 - DNA, 254 bp, Homo sapiens
tgccctccagtagagtgagattccgtctcaaaaaaaaaaaaaaaaaagaagaaatggagatacaaacttactacctacctccttacaacctaccctcacagtattactgtgaataaaagtgtgtgtagcactgggaacactattcacagagcactcatgaatgtttgttctttgttattagttactagagaggcaaatgtctgccagggctgaataatatgtgtgaattggtgattgtcgcacatatctaaaga

SEQ ID NO: 173 - DNA, 483 bp, Homo sapiens
  misc_feature (26)..(26): n is a, c, g, or t
ctcagcctcctaaggagctatttttngnattttagtagagncagggtttcnccgtgtnggncaggctggtctcgaactcctgncctcaagtgatccacccgccttggcctcccaaagtgctgggattacaggtgtgagccaccgtgcccagctagattctttactttttaataaagttcaaaatagcttttaataaggttgtcaaatttgaccaaaatttcatagaagtccaaaatattatgcctctttctgtagatcaaatgaaatatggcccaatttacaagggtatacaccttcatggtagatgaatatagattttaaactagttttaaaaggaatatgaatgatttaattgtagtttctgctgccaaaaagtctccataattattttttaataacttataactaccttgatctttacctgtacacacacacacacacacannnnnntgcacagagtaatttcacttagaatcctggtca

SEQ ID NO: 174 - DNA, 516 bp, Homo sapiens
ttaccctctatttaaatgctttgaaaaacagtgcattgacaatgggttgatatttttctttaaaagaaaaatataattatgaaagccaagataatctgaagcctgttttattttaaaactttttatgttctgtggttgatgttgtttgtttgtttgtttctattttgttggttttttactttgttttttgttttgttttgttttgttttgcatactacatgcagttctttaaccaatgtctgtttggctaatgtaattaaagttgttaatttatatgagtgcatttcaactatgtcaatggtttcttaatatttattgtgtagaagtactggtaatttttttatttacaatatgtttaaagagataacagtttgatatgttttcatgtgtttatagcagaagttatttatttctatggcattccagcggatattttggtgtttgcgaggcatgcagtcaatattttgtacagttagtggacagtattcagcaacgcctgatagcttctttggcctt

SEQ ID NO: 175 - DNA, 329 bp, Homo sapiens
gcctgacgaaacctagctattgtaagctagtttcttcagttattcttttccctatccctgtctaagctagatatatacagagaggtccagttttatgaatgcttattagatggaattttataagtctaatgcagtatcagaatagcaaatatgtgtattgcccagtttaatttactgaggtcaaattatcatgggcacttggctatcccctcctacccatgcacttatctgtaggtgtgttatcaactgatgattttctggagtttttgacccattaccatttaagatatacagagaattttatggtagtttagcccactattctgagc

SEQ ID NO: 176 - DNA, 493 bp, Homo sapiens
  misc_feature (55)..(55): n is a, c, g, or t
atttaagatctagtagccacgctaaaaaacaagtaaaattaattttaatatattnancncaatncatngnagatattattncagtaagatatttttcattcttatgaaactaagtctgtctggtttgcatatctcagttgagacactaaagttttactgataatacttaatccatgtttagatttcataaaatctaaatttgcaaagtaaatttacatacccatgttggtctaaatgtgcttaaaagttttccaataactgaaatgagtatcagttttaaatttaaaaatttgttacttggttactccatatttcaggtattagtggctaccatattggtcagagcagattcataggatgatttcccaggtttgaagtgtgtgtatatttctggtattataagatgtcagactggtttttggagaaatatgttgtcttaatttttacctgctttgatatgacctttctgtgtgtacgtgtgacatttctttat

SEQ ID NO: 177 - DNA, 156 bp, Homo sapiens
ggagggggaggtggagtggcagtgtgttccacccagcagtccctgtgtttgacctcttcgtacactctcagttgtattagctctcagaatgaaaacgagatacttttccccaatctatgtacgttgttcttaagaatgtacacttggccaggtgca

SEQ ID NO: 178 - DNA, 364 bp, Homo sapiens
gatttgaggtacaccccaagtgcctggcagctaaacacattgccattagtgccacatcatttagcttaaggctctactaagtcagtaattactttagtggcattatgagtaagtacagatgaactggcagtcgcttcaggaaagggacacatggtgagtcctgatgatgggcagggagggaaggaaggtggggtgtcgggccctgcagtggggtgtgggggagctgcagaacttggggcactagtagggtacaccatatcaggccaggaggaaaaaggagaaatcaaagttggggagtggtagggaaggaaaagggtctgtagtgaatggaacgtaactcttccctattggtgattttcctttg

SEQ ID NO: 179 - DNA, 519 bp, Homo sapiens
  misc_feature (297)..(297): n is a, c, g, or t
ggaggtttaatacggccaatggatcttgtataaagtctacgtaagttttaatttaccagagctataagatggttaaggtagactagtggggactgctacagataaaatgagatacaaagaacatttgcaaataaatgccataaaattacagtagcttggaattttagtgaatcatggcccttgtgttatctagaatgctaattattcaagctgttcctaaacttaagtatgacatataagattaaaatcttggggggaaaaactgttcagatgaaaagtcaagtcaagaagtttccncaaaagaaaagaaaaatcctaaaaaccatctagctgttgttacattaaatttatttctcacctccattaaaagggtttttgctttggagttttgttaactttcgttctttggagtaataatatttctcttgtgtatggcgctgaatacatttgtcaataatacgtcaaaaaaaaaaacacttggcttcttaatacttggaaatacgtacatattccttacta

SEQ ID NO: 180 - DNA, 341 bp, Homo sapiens
  misc_feature (46)..(46): n is a, c, g, or t
ccccatgggtagaaccttcctgattggctgtgggttgtatcaaaanacctttgactgggtgaggctgagatgggtgtggtggttccagtgagtcagnccaggttcccttccccagnccctggtccctnaggnagatgaggaccgaaaggcttaggtgacttgatggggagcncnctttcaggggccctggaagaaatgtttgtaattgttntgctttttccatgagntntggagagcacccaccaccaagccccctgaaacccaccagtccctacagcctggtctttctccccaccttcccactcccaaacacatttccacagcaaactccttgggaaggg

SEQ ID NO: 181 - DNA, 523 bp, Homo sapiens
ccttcaactggaataatttcacttcagaaaaaaaagttaagcatttccaaaacaaataaaagttgagtgtgttactaaccactggaacagtgctaaaggaaatgtaaaagaaagtctatcacgtccaaaaatgacaatatatgctgcacagcgtcataaaagcatatgaaaatagaaagctctctattaaaggtaaatacataaacagctatagaaacctctactgtcataatgatggtgcacaaaactttcacgttattgctatggaatttaaaaaatgaagcagaaagctgcataaatatgggttcatagatactcaataagaaaagataacaagtgatattaataacaaagtgggggtataaagaggtaaagctttgcattccattgaaatatagtcattatatattgtcataactttaagatgttttatgaagtctttatttctcaagatgattacaaaaaaacctgtagatggtatgcagaagcaaatgagaaagaaattgaatcatgtcactacaaa

SEQ ID NO: 182 - DNA, 255 bp, Homo sapiens
  misc_feature (59)..(59): n is a, c, g, or t
gcatagttaagtaaatcaccccaattttttctaatatttaagtgaacatttaaagatangcatttcttaatgttaatttcttttctttcatgtaatcttgtcagtagacaaatgatggtgactatattctgtaataagtacatatattatgattaaaaaaaaaagcgtgtactatctgcccaaagagatcataagcactttggagctataaacttcttgtctatttctacttccatggtactgatattctcttct